SEQUENCE LISTING (70 sequences)

SEQ ID NO: 1 - protein, 10 aa, Artificial Sequence
  Synthetic peptide
GFTFSDYWMH

SEQ ID NO: 2 - protein, 19 aa, Artificial Sequence
  Synthetic peptide
HIKSKTDAGTTDYAAPVKG

SEQ ID NO: 3 - protein, 9 aa, Artificial Sequence
  Synthetic peptide
EIYYYAFDS

SEQ ID NO: 4 - protein, 5 aa, Artificial Sequence
  Synthetic peptide
DYWMH

SEQ ID NO: 5 - protein, 7 aa, Artificial Sequence
  Synthetic peptide
GFTFSDY

SEQ ID NO: 6 - protein, 8 aa, Artificial Sequence
  Synthetic peptide
KSKTDAGT

SEQ ID NO: 7 - protein, 120 aa, Artificial Sequence
  Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSS

SEQ ID NO: 8 - DNA, 360 bp, Artificial Sequence
  Synthetic polynuceotide
gaggtgcagctggtggaatcaggcggcggactggtcaagcctggcggtagcctgagactgagctgcgctgctagtggcttcacctttagcgactactggatgcactgggtcagacaggcccctggtaaaggcctggagtgggtcggacacattaagtctaagaccgacgccggcactaccgactacgccgctcctgtgaagggccggttcactatctctagggacgactctaagaacaccctgtacctgcagatgaatagcctgaaaaccgaggacaccgccgtctactactgcgctagagagatctactactacgccttcgatagctggggtcagggcaccctggtcaccgtgtctagc

SEQ ID NO: 9 - protein, 223 aa, Artificial Sequence
  Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - DNA, 669 bp, Artificial Sequence
  Synthetic polynuceotide
gaggtgcagctggtggaatcaggcggcggactggtcaagcctggcggtagcctgagactgagctgcgctgctagtggcttcacctttagcgactactggatgcactgggtcagacaggcccctggtaaaggcctggagtgggtcggacacattaagtctaagaccgacgccggcactaccgactacgccgctcctgtgaagggccggttcactatctctagggacgactctaagaacaccctgtacctgcagatgaatagcctgaaaaccgaggacaccgccgtctactactgcgctagagagatctactactacgccttcgatagctggggtcagggcaccctggtcaccgtgtctagcgctagcactaagggcccctccgtgttccctctggccccttccagcaagtctacctctggcggcaccgctgctctgggctgcctggtgaaggactacttccctgagcctgtgacagtgtcctggaactctggcgccctgacctccggcgtgcacaccttccctgccgtgctgcagtcctccggcctgtactccctgtcctccgtggtgacagtgccttcctccagcctgggcacccagacctatatctgcaacgtgaaccacaagccttccaacaccaaggtggacaagcgggtggagcctaagtcatgc

SEQ ID NO: 11 - protein, 11 aa, Artificial Sequence
  Synthetic peptide
SGDNIGSKYVH

SEQ ID NO: 12 - protein, 7 aa, Artificial Sequence
  Synthetic peptide
GDNERPS

SEQ ID NO: 13 - protein, 10 aa, Artificial Sequence
  Synthetic peptide
QAADWVDFYV

SEQ ID NO: 14 - protein, 7 aa, Artificial Sequence
  Synthetic peptide
DNIGSKY

SEQ ID NO: 15 - protein, 3 aa, Artificial Sequence
  Synthetic peptide
GDN

SEQ ID NO: 16 - protein, 7 aa, Artificial Sequence
  Synthetic peptide
ADWVDFY

SEQ ID NO: 17 - protein, 107 aa, Artificial Sequence
  Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVL

SEQ ID NO: 18 - DNA, 321 bp, Artificial Sequence
  Synthetic polynuceotide
agctacgagctgactcagcccctgagcgtcagcgtggccctgggtcagaccgctagaatcacctgtagcggcgataatatcggctctaaatacgtgcactggtatcagcagaagcccggtcaggcccccgtgctggtgatctacggcgataacgagcggcctagcggaatccccgagcggtttagcggctctaatagcggtaacaccgctaccctgactatctctagggctcaggccggcgacgaggccgactactactgtcaggccgccgactgggtggacttctacgtgttcggcggaggcactaagctgaccgtgctg

SEQ ID NO: 19 - protein, 213 aa, Artificial Sequence
  Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - DNA, 639 bp, Artificial Sequence
  Synthetic polynuceotide
agctacgagctgactcagcccctgagcgtcagcgtggccctgggtcagaccgctagaatcacctgtagcggcgataatatcggctctaaatacgtgcactggtatcagcagaagcccggtcaggcccccgtgctggtgatctacggcgataacgagcggcctagcggaatccccgagcggtttagcggctctaatagcggtaacaccgctaccctgactatctctagggctcaggccggcgacgaggccgactactactgtcaggccgccgactgggtggacttctacgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 21 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 22 - DNA, 2629 bp, Homo sapiens
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaacattaactctaactgtgacaaagaagaccacaaatagttatcttttaattacagaagagtttcttaacttacttttgtaagtttttattgtgtaagtttataatgcaggggaagtactactcctcaaatgttgagggaagcttccataacattgatgactggcttcatggcagtaattctcggctgtagttgcataagcattgctcaagaggaaaatccaaaagtgcagcaggagaactcttttccctgaaaaaggaaaaatattgaactcaatgatagcacctaaacttacatttaaaagacagacattccttctacatgtaatgacacttcttgtgttaaactaaaaatttacaagagaagaaagtgaaagcaaatggggtttcacaaatagttgtaaatatagtgaagcaatttgaaataattttcaagcaaagtattgtgaaagtattctaagccaagttttaaatattatctaacagacaagagtggtatatacaagtagatcctgagaagtacctttgttacagctactataaatatacatataaattatagaatctactttaatttattttgtgaacacttttgaaaatgtacatgttcctttgtaattgacactatatatttcttaataaaataattctcaaatttgtttcttatgaatcatctctcaaatctagttagacaatttgcacacatacttttctaagggacattatcttccttcaggtttttacctccactcatccttagagcccactgactgctcccctttatacctgttggccctgcctataggagagaatatttggagataggcagcttcaggatgcattgcaatcatccttttcttaaattatgtcactagtcttttattttttcccctcttgaactttcctcacacctggaagaaacaaagtaggaaaaagtgaacaggggatgtcaaatcgattcttgaattcccgctgcaagctagagccgcaggcaccctctcactcaatttccactcagaaccctataaacaccagtgggaagggcaacccactgcacgtgggaatgcactgatttttcctaggagtagacatgttcctctaattactccctgagggttagttggggctaaaccatgacagaagtggggaagttcaatgtccttaaatccatcttacttgccaacaggtaagaggaagcttacattacatgtccagtccacatttaaagagcacttactgtggaacaagccttcagccaaacaatggggatagaaaagtaggtaagactcagcctttgtccagagaagctcagggtatagctgaataggcagtttcttttgtcctgaggaaaatcaggacatgcctgctttctaaaaatcttcctctgaagacctgacccaagctcttaaatgctattgtaagagaaatttctttgtctattaactccattttagtagggattcactgactagattttactgaactatgaaaataaatacacataatttttcacaaaattttgggcccaattcccctaaaagaattgaggattagggagaaaggagacaactcaaagtcatcccattaagtgcagtttctttgaatcttctgctttatctttaaaaatttgtataatttatatattttattctatgtgttccatagatatcttaatgtaaaattagtcatttaaattacactgtcaattaaaagtaatgggcaagagattgcatcatactaatttagtaagaacgttcccaaatgttgtaacaatgtggatcatacatctctggttttttaaatgtattgaggctttcttggtggactagtatagtatacggtcagttatgtcaatgtttcatggtcaataaaaaggaagttgcaaattgt

SEQ ID NO: 23 - protein, 63 aa, Homo sapiens
MFAMKTKAALAIWCPGYSETQINATQAMKKRRKRKVTTNKCLEQVSQLQGLWRRFNRPLLKQQ

SEQ ID NO: 24 - DNA, 2411 bp, Homo sapiens
accctcgccacgcccctgctcccccgcggttggttcttccttgctctactcaaccctgacctcttctctctgactctcgacttgtgttccccgctcctccctgaccttcctcccctcccctttcactcaattctcaccaactctttctctctctggtgttttctccttttctcgtaaactttgccgcctatgagcagccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaacattaactctaactgtgacaaagaagaccacaaatagttatcttttaattacagaagagtttcttaacttacttttgtaagtttttattgtgtaagtttataatgcaggggaagtactactcctcaaatgttgagggaagcttccataacattgatgactggcttcatggcagtaattctcggctgtagttgcataagcattgctcaagaggaaaatccaaaagtgcagcaggagaactcttttccctgaaaaaggaaaaatattgaactcaatgatagcacctaaacttacatttaaaagacagacattccttctacatgtaatgacacttcttgtgttaaactaaaaatttacaagagaagaaagtgaaagcaaatggggtttcacaaatagttgtaaatatagtgaagcaatttgaaataattttcaagcaaagtattgtgaaagtattctaagccaagttttaaatattatctaacagacaagagtggtatatacaagtagatcctgagaagtacctttgttacagctactataaatatacatataaattatagaatctactttaatttattttgtgaacacttttgaaaatgtacatgttcctttgtaattgacactatatatttcttaataaaataattctcaaatttgtttcttatgaatcatctctcaaatctagttagacaatttgcacacatacttttctaagggacattatcttccttcaggtttttacctccactcatccttagagcccactgactgctcccctttatacctgttggccctgcctataggagagaatatttggagataggcagcttcaggatgcattgcaatcatccttttcttaaattatgtcactagtcttttattttttcccctcttgaactttcctcacacctggaagaaacaaagtaggaaaaagtgaacaggggatgtcaaatcgattcttgaattcccgctgcaagctagagccgcaggcaccctctcactcaatttccactcagaaccctataaacaccagtgggaagggcaacccactgcacgtgggaatgcactgatttttcctaggagtagacatgttcctctaattactccctgagggttagttggggctaaaccatgacagaagtggggaagttcaatgtccttaaatccatcttacttgccaacaggtaagaggaagcttacattacatgtccagtccacatttaaagagcacttactgtggaacaagccttcagccaaacaatggggatagaaaagtaggtaagactcagcctttgtccagagaagctcagggtatagctgaataggcagtttcttttgtcctgaggaaaatcaggacatgcctgctttctaaaaatcttcctctgaagacctgacccaagctcttaaatgctattgtaagagaaatttctttgtctattaactccattttagtagggattcactgactagattttactgaactatgaaaataaatacacataatttttcacaaaattttgggcccaattcccctaaaagaattgaggattagggagaaaggagacaactcaaagtcatcccattaagtgcagtttctttgaatcttctgctttatctttaaaaatttgtataatttatatattttattctatgtgttccatagatatcttaatgtaaaattagtcatttaaattacactgtcaattaaaagtaatgggcaagagattgcatcatactaatttagtaagaacgttcccaaatgttgtaacaatgtggatcatacatctctggttttttaaatgtattgaggctttcttggtggactagtatagtatacggtcagttatgtcaatgtttcatggtcaataaaaaggaagttgcaaattgt

SEQ ID NO: 25 - protein, 11 aa, Artificial Sequence
  Synthetic peptide
GGNNLGSKSVH

SEQ ID NO: 26 - protein, 7 aa, Artificial Sequence
  Synthetic peptide
DDSDRPS

SEQ ID NO: 27 - protein, 11 aa, Artificial Sequence
  Synthetic peptide
QVWDSSSDHVV

SEQ ID NO: 28 - protein, 5 aa, Artificial Sequence
  Synthetic peptide
TYGMH

SEQ ID NO: 29 - protein, 16 aa, Artificial Sequence
  Synthetic peptide
VIWYDGSNKHYADSVK

SEQ ID NO: 30 - protein, 13 aa, Artificial Sequence
  Synthetic peptide
APQWELVHEAFDI

SEQ ID NO: 31 - protein, 108 aa, Artificial Sequence
  Synthetic peptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 32 - protein, 122 aa, Artificial Sequence
  Synthetic peptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 33 - protein, 448 aa, Artificial Sequence
  Synthetic peptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 214 aa, Artificial Sequence
  Synthetic peptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 107 aa, Artificial Sequence
  Synthetic peptide
DIQMTQSPSSLSASVGDRVTITCRASRPIRNWLDWYQQKPGKAPKLLIWGASHLQSGVPSRFSGSGSGTDFTLTISSLQPEDFATYYCVQIGEDPVTFGQGTKVEIK

SEQ ID NO: 36 - protein, 11 aa, Artificial Sequence
  Synthetic peptide
RASRPIRNWLD

SEQ ID NO: 37 - protein, 7 aa, Artificial Sequence
  Synthetic peptide
GASHLQS

SEQ ID NO: 38 - protein, 10 aa, Artificial Sequence
  Synthetic peptide
LLIWGASHLQ

SEQ ID NO: 39 - protein, 9 aa, Artificial Sequence
  Synthetic peptide
VQIGEDPVT

SEQ ID NO: 40 - protein, 12 aa, Artificial Sequence
  Synthetic peptide
GFSITTSGYYWT

SEQ ID NO: 41 - protein, 12 aa, Artificial Sequence
  Synthetic peptide
GFSITTSGYYWS

SEQ ID NO: 42 - protein, 16 aa, Artificial Sequence
  Synthetic peptide
YIGYNSKTYYNPSLKS

SEQ ID NO: 43 - protein, 16 aa, Artificial Sequence
  Synthetic peptide
YIGYNSKTYYNPALKS

SEQ ID NO: 44 - protein, 12 aa, Artificial Sequence
  Synthetic peptide
SLYGGYKDAFDS

SEQ ID NO: 45 - protein, 11 aa, Artificial Sequence
  Synthetic peptide
KASQSIGTSLH

SEQ ID NO: 46 - protein, 11 aa, Artificial Sequence
  Synthetic peptide
RASQSIGTSLH

SEQ ID NO: 47 - protein, 7 aa, Artificial Sequence
  Synthetic peptide
FASRSIT

SEQ ID NO: 48 - protein, 7 aa, Artificial Sequence
  Synthetic peptide
FASRLQS

SEQ ID NO: 49 - protein, 7 aa, Artificial Sequence
  Synthetic peptide
FASRSIS

SEQ ID NO: 50 - protein, 9 aa, Artificial Sequence
  Synthetic peptide
QQSPGFPPT

SEQ ID NO: 51 - protein, 122 aa, Artificial Sequence
  Synthetic peptide
QIQLQESGPGLVKPSQSLSLTCSVTGFSITTSGYYWTWIRQFPGKKLEWMGYIGYNSKTYYNPSLKSRISITRDTSKNQFLLHLNSVTTEDTATYYCARSLYGGYKDAFDSWGQGTLVTVSS

SEQ ID NO: 52 - protein, 108 aa, Artificial Sequence
  Synthetic peptide
DVVLTQTPATLSAIPGERVTMTCKASQSIGTSLHWYQHRPNETPRLLIKFASRSITGIPSRFSGSGSGTDFTLGINNLEAEDFAIYYCQQSPGFPPTFGSGTKLEINR

SEQ ID NO: 53 - protein, 122 aa, Artificial Sequence
  Synthetic peptide
QVQLVESGGGLVKPGGSLRLSCAASGFSITTSGYYWTWIRQFPGKGLEWMGYIGYNSKTYYNPALKSRITISRDNAKNSLYLQMNSLRAEDTAVYYCARSLYGGYKDAFDSWGQGTLVTVSS

SEQ ID NO: 54 - protein, 108 aa, Artificial Sequence
  Synthetic peptide
DIQMTQSPSSLSASVGDRVTITCRASQSIGTSLHWYQQKPGKAPKLLIKFASRLQSGVPSRFSGSGSGTDFTLTISSLQPEDFATYYCQQSPGFPPTFGQGTKLEIKR

SEQ ID NO: 55 - protein, 122 aa, Artificial Sequence; identical to SEQ ID NO: 53
  Synthetic peptide

SEQ ID NO: 56 - protein, 108 aa, Artificial Sequence
  Synthetic peptide
EIVLTQSPDFQSVTPKEKVTITCRASQSIGTSLHWYQQKPDQSPKLLIKFASRSISGVPSRFSGSGSGTDFTLTINSLEAEDAATYYCQQSPGFPPTFGQGTKLEIKR

SEQ ID NO: 57 - protein, 122 aa, Artificial Sequence
  Synthetic peptide
QVQLQESGPGLVKPSQTLSLTCTVSGFSITTSGYYWSWIRQHPGKGLEWIGYIGYNSKTYYSPSLKSRVTISRDTSKNQFSLKLSSVTAADTAVYYCARSLYGGYKDAFDSWGQGTLVTVSS

SEQ ID NO: 58 - protein, 108 aa, Artificial Sequence
  Synthetic peptide
DIQMTQSPSSLSASVGDRVTITCRASQSIGTSLHWYQHRPGETPKLLIKFASRSISGVPSRFSGSGSGTDFTLTISSLQPEDFATYYCQQSPGFPPTFGQGTKLEIKR

SEQ ID NO: 59 - protein, 122 aa, Artificial Sequence; identical to SEQ ID NO: 53
  Synthetic peptide

SEQ ID NO: 60 - protein, 108 aa, Artificial Sequence
  Synthetic peptide
EIVMTQSPATLSVSPGERATLSCRASQSIGTSLHWYQQKPGQAPRLLIKFASRSITGIPARFSGSGSGTEFTLTISSLQSEDFAVYYCQQSPGFPPTFGQGTKLEIKR

SEQ ID NO: 61 - protein, 10 aa, Artificial Sequence
  Synthetic peptide
GYIFTDYAMH

SEQ ID NO: 62 - protein, 16 aa, Artificial Sequence
  Synthetic peptide
TFIPLLDTSDYAQKFQ

SEQ ID NO: 63 - protein, 11 aa, Artificial Sequence
  Synthetic peptide
MGVTHSYVMDA

SEQ ID NO: 64 - protein, 11 aa, Artificial Sequence
  Synthetic peptide
RASQPISISVH

SEQ ID NO: 65 - protein, 7 aa, Artificial Sequence
  Synthetic peptide
FASQSIS

SEQ ID NO: 66 - protein, 9 aa, Artificial Sequence
  Synthetic peptide
QQTFSLPYT

SEQ ID NO: 67 - protein, 120 aa, Artificial Sequence
  Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSS

SEQ ID NO: 68 - protein, 109 aa, Artificial Sequence
  Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRT

SEQ ID NO: 69 - protein, 450 aa, Artificial Sequence
  Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 214 aa, Artificial Sequence
  Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0082287 A1
(43) Pub. Date: Mar. 16, 2023

(54) METHODS OF TREATING AN INFLAMMATORY OR OBSTRUCTIVE AIRWAY DISEASE USING ANTI-TSLP ANTIBODY

(71) Applicant: Novartis AG, Basel (CH)

(72) Inventors: Ankur AJMERA, Basel (CH); Jürgen DEDERICHS, Hartheim am Rhein (DE); Sarah Schmidt GRANT, Basel (CH); Kristin HENSON, Cambridge, MA (US); Monish JAIN, Belmont, MA (US); Sandro PAGANI, Zeifen (CH); Yinuo PANG, Lincolnshire, IL (US); Marianna ROWLANDS, Cambridge, MA (US)

(21) Appl. No.: 17/759,538

(22) PCT Filed: Jan. 28, 2021
(86) PCT No.: PCT/IB2021/050651

§ 371 Date: Jul. 27, 2022

Related U.S. Application Data
(60) Provisional application No. 62/967,313, filed on Jan. 29, 2020.
(60) Provisional application No. 63/031,520, filed on May 28, 2020.

(51) Int. Cl. C07K 16/24; A61K 9/00; A61P 11/00; A61K 45/06

(52) U.S. Cl. CPC C07K 16/24; A61K 9/0073; A61P 11/00; A61K 45/06; A61K 2039/545

(57) ABSTRACT

The present disclosure relates to methods, treatment regimens, uses, kits, compositions and medicaments for treating an inflammatory or obstructive airway disease, such as asthma, including severe asthma, or COPD, by employing an anti-TSLP antibody or anti-TSLP antibody fragment.

### SEQUENCE LISTING

The instant application contains a Sequence Listing which has been submitted electronically in ASCII format and is hereby incorporated by reference in its entirety. Said ASCII copy, created on Nov. 10, 2020, is named PAT058671_SEQ LISTING_ST25.txt and is 53.1 KB in size.

### TECHNICAL FIELD

The present disclosure relates to methods, treatment regimens, uses, kits, compositions and medicaments for treating an inflammatory or obstructive airway disease, such as asthma, including severe asthma, or COPD, by employing an anti-TSLP antibody or anti-TSLP antibody fragment.

### BACKGROUND

Asthma is characterized by chronic airway inflammation with symptoms of wheeze, shortness of breath, chest tightness and cough that vary over time and intensity. In addition, patients show variable airflow limitation. About 300 million people worldwide suffer from asthma and there are approximately 250,000 deaths caused by asthma every year (D'Amato et al 2016). While mild forms of asthma are generally well controlled with currently available therapy (inhaled bronchodilators, leukotriene receptor antagonists, oral and inhaled corticosteroids), approximately 10% of asthma patients are refractory to standard therapy. While a subset of these patients may respond to anti-immunoglobulin (Ig)E or anti-interleukin (IL)-5 therapy, many severe asthma patients continue to experience significant asthma-related symptoms. Disease modifying therapy for these severe asthma patients would address significant unmet medical need.

In the case of asthma with type 2 inflammation, characterized by eosinophilic inflammation and evidence of atopy, T helper type 2 (Th2) immune pathway elements are crucial in the development and maintenance of airway inflammation and airway hyperresponsiveness. A key upstream regulator of the Th2 response is thymic stromal lymphopoietin (TSLP) (He and Geha, 2010, Ann N Y Acad Sci. 1183:13-24). The role of TSLP is to modulate dendritic cells and induce the differentiation of naive T cells into inflammatory Th2 cells and promote cytokine secretion from mast cells, eosinophils and macrophages as part of the innate immune response. High level of TSLP expression has been found in asthmatic lung epithelial cells and chronic atopic dermatitis lesions, suggesting a role for TSLP in allergic inflammation (Ziegler and Artis, 2010, Nature immunology 11, 289-293). More recent evidence implicates TSLP in the differentiation of Th17 cells and Th17-driven inflammatory processes (Hartgring et al., 2011, Arthritis and rheumatism 63, 1878-1887; Tanaka et al., 2009, Clinical and experimental allergy: Journal of the British Society for Allergy and Clinical Immunology 39, 89-100; Wu et al., 2014, Journal of molecular and cellular cardiology 76, 33-45). Chronic allergic (atopic) asthma is often characterized by Th2-type inflammation, while non-allergic asthmatic inflammation is predominately neutrophilic with a mixed Th1 and Th17 cytokine milieu. The consequences of chronic inflammation in asthma include bronchial hyper-reactivity (BHR), mucus overproduction, airway wall remodeling and airway narrowing (Lambrecht and Hammad, 2014, Nature immunology 16, 45-56). TSLP was shown to be involved in the initiation and maintenance/enhancement of the allergic asthmatic response (Wang et al., 2006, Immunity 24, 827-838). More recently, TSLP signaling was also found to be required for the recall response of memory T-cells to local antigen challenge (Wang et al., 2015, The Journal of allergy and clinical immunology 135, 781-791 e783).

TSLP is predominantly expressed in the airway epithelium of asthmatic patients. In asthma patients, an increase in TSLP protein levels has been observed in both lung tissue and bronchial alveolar lavage (BAL) fluid, and TSLP levels correlate with disease severity (Ying et al 2008, Ying et al 2005). In addition, increased TSLP expression has been observed in the airway lamina propria of severe asthma patients (Ferreira et al 2012).

Despite available treatment for asthma, there remains a high medical need for new treatment options for asthma subjects. CSJ117 is a potent neutralizing antibody fragment (fragment antigen-binding, Fab) directed against human TSLP (WO2017/042701). CSJ117, as an inhaled Fab targeting TSLP, offers the potential to be an efficacious therapy for an inflammatory or obstructive airway disease, in particular asthma, more particularly, severe asthma, with a favorable safety and tolerability profile. The present invention relates to the use of CSJ117 in the treatment of an inflammatory or obstructive airway disease, such as asthma, in particular severe asthma, or COPD. The present invention relates to a dosage regime, which the inventors have found to be efficacious in the treatment of airway disease.

### SUMMARY OF THE INVENTION

In a first aspect, the invention relates to a method of treating an inflammatory or obstructive airway disease comprising administering to a subject in need thereof a dose of about 2 mg to about 16 mg of an anti-TSLP antibody or anti-TSLP antibody fragment.

In a second aspect, the invention relates to a method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, comprising administering to a subject in need thereof a dose of about 2 mg to about 16 mg of an anti-TSLP antibody or anti-TSLP antibody fragment.

In a third aspect, the invention relates to a method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or anti-TSLP antibody fragment, wherein the subject has a non-eosinophilic profile or a low eosinophil profile.

In a fourth aspect, the invention relates to a method for treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or antibody variant, wherein the antibody or anti-TSLP antibody fragment, wherein the subject has a Th2 low profile.

In a fifth aspect, the invention relates to an anti-TSLP antibody or anti-TSLP antibody fragment for use in the treatment of an inflammatory or obstructive airway disease, wherein the anti-TSLP antibody or anti-TSLP antibody fragment is administered to a subject at a dose of about 0.5 mg to about 16 mg. In addition, or alternatively, the invention relates to use of an anti-TSLP antibody or anti-TSLP antibody fragment for the manufacture of a medicament for treating of an inflammatory or obstructive airway disease, wherein the anti-TSLP antibody or anti-TSLP antibody fragment is administered to a subject at a dose of about 2 mg to about 16 mg.

In a sixth aspect, the invention relates to an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a non-eosinophilic profile or a low eosinophil profile. In addition, or alternatively, the invention relates to use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a non-eosinophilic profile or a low eosinophil profile.

In a seventh aspect, the invention relates to an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score or improving asthma or COPD symptoms in a subject in a subject, wherein the subject has a Th2 low profile. In addition, or alternatively, the invention relates to use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a Th2 low profile.

In an eighth aspect, the invention relates to an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, wherein the anti-TSLP antibody or anti-TSLP antibody fragment is administered to a subject at a dose of about 0.5 mg to about 16 mg. In addition, or alternatively, the invention relates to use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, wherein the anti-TSLP antibody or anti-TSLP antibody fragment is administered to a subject at a dose of about 2 mg to about 16 mg.

In a ninth aspect, the invention relates to a medicament for administration through inhalation comprising an anti-TSLP antibody or anti-TSLP antibody fragment, in particular an anti-TSLP antibody fragment, more particularly an anti-TSLP antibody Fab, wherein the medicament comprises 0.5 mg to 16 mg of said anti-TSLP antibody or anti-TSLP antibody fragment. In one embodiment, the invention relates to a medicament for administration through inhalation comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the medicament, in particular about 3% to about 50% (w/w) of the medicament, and wherein the medicament comprises 2 mg to 16 mg of said anti-TSLP antibody or anti-TSLP antibody fragment.

In a tenth aspect, the invention relates to a pharmaceutical composition comprising spray-dried particles comprising: (i) a core comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the composition, in particular about 3% to about 50% (w/w) of the composition; and (ii) a shell comprising trileucine, wherein trileucine is about 1% to about 25% (w/w), in particular about 10% to about 15% (w/w) of the composition, preferably 10% w/w.

In an eleventh aspect, the invention relates to a kit comprising the pharmaceutical composition of the invention or the medicament of the invention, and a device for delivering the pharmaceutical composition or the medicament to a subject.

In a twelfth aspect the invention relates to the pharmaceutical composition or the medicament of the invention for use in the treatment of an inflammatory or obstructive airway disease.

Non-limiting embodiments of the present disclosure are described in the following embodiments:
Embodiment 1. A method of treating an inflammatory or obstructive airway disease comprising administering to a subject in need thereof a dose of about 0.5 mg to about 16 mg, in particular about 2 mg to about 16 mg, more particularly about 4 mg to about 16 mg, of an anti-TSLP antibody or anti-TSLP antibody fragment.
Embodiment 2. A method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject, comprising administering to a subject in need thereof a dose of about 0.5 mg to about 16 mg, in particular about 2 mg to about 16 mg, more particularly about 4 mg to about 16 mg, of an anti-TSLP antibody or anti-TSLP antibody fragment.
Embodiment 3. A method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or anti-TSLP antibody fragment, wherein the subject has a non-eosinophilic profile or a low eosinophil profile.
Embodiment 4. A method for treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or antibody variant, wherein the antibody or anti-TSLP antibody fragment, wherein the subject has a Th2 low profile.
Embodiment 5. The method of any one of embodiments 1 to 4, wherein the antibody or antibody fragment is administered at a dose of:
   a) about 0.5 mg, in particular 0.5 mg; or
   b) about 1 mg, in particular 1 mg; or
   c) about 2 mg, in particular 2 mg; or
   d) about 4 mg, in particular 4 mg; or
   e) about 8 mg, in particular 8 mg; or
   f) about 16 mg, in particular 16 mg.
Embodiment 6. The method of any one of embodiments 1 to 5, wherein the dose is a daily dose.
Embodiment 7. The method of any one of embodiments 1 to 6, wherein the antibody or antibody fragment is administered at said dose once a day.
Embodiment 8. The method of any one of embodiments 1 to 7, wherein the antibody or antibody fragment is administered for a period of at least 1 month, at least 2 months, at least 3 months, at least 4 months, at least 6 months, at least 9 months, at least 1 year or more.
Embodiment 9. The method of any one of embodiments 1 to 8, wherein the antibody or antibody fragment is administered to the subject orally or intranasally.
Embodiment 10. The method of embodiment 9, wherein the antibody or antibody fragment is administered to the subject in an aerosolized form.
Embodiment 11. The method of embodiment 9 or embodiment 10, wherein the antibody or antibody fragment is administered to the subject by inhalation.
Embodiment 12. The method of embodiment 11, wherein the antibody or antibody fragment is administered to the subject by using a dry powder inhaler.
Embodiment 13. The method according to any one of embodiments 1 to 12, wherein the subject has allergic asthma or non-allergic asthma.
Embodiment 14. The method according to any one of embodiments 1 to 13, wherein the subject has mild asthma.
Embodiment 15. The method according to any one of embodiments 1 to 13, wherein the subject has moderate asthma.
Embodiment 16. The method according to any one of embodiments 1 to 13, wherein the subject has moderate-to-severe asthma or severe asthma.
Embodiment 17. The method according to embodiment 15 or 16, wherein the subject has uncontrolled asthma, in particular severe uncontrolled asthma.
Embodiment 18. The method according to any one of the preceding embodiments, wherein the subject has eosinophilic or non-eosinophilic asthma.
Embodiment 19. The method according to any one of embodiments 1 to 18, wherein the subject has eosinophilic or non-eosinophilic COPD.
Embodiment 20. The method of embodiment 18 or 19, wherein the subject has high eosinophil count, e.g., ≥250 cells/μl, particularly >300 cells/μl, in particular at the time of start of treatment or at the time of diagnosis.
Embodiment 21. The method of embodiment 18 or 19, wherein the subject has low eosinophil count, e.g., <200 cells/μl, particularly <300 cells/μl, in particular at the time of start of treatment or at the time of diagnosis.
Embodiment 22. The method according to any one of embodiments 1 to 17 or 21, wherein the subject has Th2 low profile.
Embodiment 23. The method of embodiment 22, wherein the subject has a Th2 profile of IgE less than or equal to 100 IU/ml or eosinophil count of less than 140 cells/μl, in particular at the time of start of treatment or at the time of diagnosis.
Embodiment 24. The method according to any one of embodiments 1 to 23, wherein the subject is an adult or adolescent.
Embodiment 25. The method according to any one of embodiments 1 to 23, wherein the subject is a child.
Embodiment 26. The method according to any one of the preceding embodiments, wherein the subject is on background therapy of:
   (a) medium or high dose corticosteroids, e.g., inhaled corticosteroids (ICS); or
   (b) medium or high dose corticosteroids, e.g., inhaled corticosteroids (ICS), in combination with LABA (ICS/LABA); or
   (c) medium or high dose corticosteroids, e.g., inhaled corticosteroids (ICS), in combination with LABA and up to two additional to ICS-LABA controllers, e.g., LTRA, theophylline or its derivatives or LAMA, e.g., ICS/LABA/LAMA).
Embodiment 27. The method according to any one of the preceding embodiments, wherein the subject is selected according to at least one of the following criteria:
   a) prior to treatment with the antibody or antibody fragment, the subject has a post-bronchodilator reversibility of FEV1≥12% and ≥200 mL;
   b) prior to treatment with the antibody or antibody fragment, the subject has a spirometry with pre-bronchodilator FEV1≥40% and ≤85%;
   c) prior to treatment with the antibody or antibody fragment, the subject has an ACQ-5 score ≥1.5.
Embodiment 28. The method according to any one of the preceding embodiments, wherein said subject achieves by week 12, preferably by week 4 or week 8, of the treatment at least one of the following:
   a) an improvement in FVC;
   b) an improvement in the FEV1;
   c) an improvement of FeNO
   d) an improvement in morning and evening peak expiratory flow rate (PEF);
   e) an improvement of one or more symptoms of asthma, e.g., a reduction of the daytime and nighttime asthma symptom score (ADSD and/or ANSD), as measured by an asthma symptom diary;
   f) a reduction in the ACQ-5 score;
   g) a reduction of Asthma Quality of Life Questionnaire (AQLQ) score, e.g., AQLQ+12 score.
Embodiment 29. The method of any of the preceding embodiments, wherein the administration decreases eosinophils in blood, sputum, broncheoalveolar fluid, or lungs of the subject.
Embodiment 30. The method of any of the preceding embodiments, wherein the administration decreases Th2 cell count, in particular wherein the administration shifts cell counts in the subject from a Th2 high population to a Th2 low population.
Embodiment 31. The method of any of the preceding embodiments, wherein the administration delays the time to an asthma or COPD exacerbation compared to a subject not receiving the anti-TSLP antibody or antibody fragment thereof.
Embodiment 32. The method of any of the preceding embodiments, wherein the anti-TSLP antibody or anti-TSLP antibody fragment is selected from any one of the following:
   a) an antibody or antibody fragment that comprises:
      a HCDR1 comprising the amino acid sequence of SEQ ID NO: 4;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3) comprising the amino acid sequence of SEQ ID NO: 13;
   b) an antibody or antibody fragment that comprises:
      a HCDR1 comprising the amino acid sequence of SEQ ID NO: 5;
      a HCDR2 comprising the amino acid sequence of SEQ ID NO: 6;
      a HCDR3 comprising the amino acid sequence of SEQ ID NO: 3;
      a LCDR1 comprising the amino acid sequence of SEQ ID NO: 14;
      a LCDR2 comprising the amino acid sequence of SEQ ID NO: 15; and
      a LCDR3 comprising the amino acid sequence of SEQ ID NO: 16; and
   c) an antibody or antibody fragment that comprises
      a HCDR1 comprising the amino acid sequence of SEQ ID NO: 1;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3 comprising the amino acid sequence of SEQ ID NO: 13.
Embodiment 33. The method of embodiment 32, wherein the anti-TSLP antibody or anti-TSLP antibody fragment comprises
   a) a heavy chain variable region comprising
      i. the amino acid sequence of SEQ ID NO: 7, or
      ii. a sequence at least 90% identical to SEQ ID NO: 7, or
      iii. a conservative variant of SEQ ID NO: 7; and
   b) a light chain variable region comprising
      i. the amino acid sequence of SEQ ID NO: 17, or
      ii. a sequence at least 90% identical to SEQ ID NO: 17, or
      iii. a conservative variant of SEQ ID NO: 17.
Embodiment 34. The method of embodiment 32 or embodiment 33, wherein the anti-TSLP antibody or anti-TSLP antibody fragment comprises
   a) a heavy chain comprising
      i. the amino acid sequence of SEQ ID NO: 9, or
      ii. a sequence at least 90% identical to SEQ ID NO: 9, or
      iii. a conservative variant of SEQ ID NO: 9, and
   b) a light chain comprising
      i. the amino acid sequence of SEQ ID NO: 19, or
      ii. a sequence at least 90% identical to SEQ ID NO: 19, or
      iii. a conservative variant of SEQ ID NO: 19.
Embodiment 35. The method of any of the embodiments 1 to 31, wherein the anti-TSLP antibody or anti-TSLP antibody fragment comprises: a HCDR1 comprising the amino acid sequence of SEQ ID NO: 28; a HCDR2 comprising the amino acid sequence of SEQ ID NO: 29; a HCDR3 comprising the amino acid sequence of SEQ ID NO: 30; a LCDR1 comprising the amino acid sequence of SEQ ID NO: 25; a LCDR2 comprising the amino acid sequence of SEQ ID NO: 26; and a LCDR3 comprising the amino acid sequence of SEQ ID NO: 27.
Embodiment 36. The method of embodiment 35, wherein the anti-TSLP antibody or anti-TSLP antibody fragment comprises:
   a) a heavy chain variable region comprising (or consisting of)
      i. the amino acid sequence of SEQ ID NO: 32, or
      ii. a sequence at least 90% identical to SEQ ID NO: 32, or
      iii. a conservative variant of SEQ ID NO: 32; and
   b) a light chain variable region comprising (or consisting of)
      i. the amino acid sequence of SEQ ID NO: 31, or
      ii. a sequence at least 90% identical to SEQ ID NO: 31, or
      iii. a conservative variant of SEQ ID NO: 31.
Embodiment 37. The method of embodiment 35 or embodiment 36, wherein the anti-TSLP antibody or anti-TSLP antibody fragment comprises:
   a) a heavy chain comprising (or consisting of)
      i. the amino acid sequence of SEQ ID NO: 33, or
      ii. a sequence at least 90% identical to SEQ ID NO: 33, or
      iii. a conservative variant of SEQ ID NO: 33, and
   b) a light chain comprising (or consisting of)
      i. the amino acid sequence of SEQ ID NO: 34, or
      ii. a sequence at least 90% identical to SEQ ID NO: 34, or
      iii. a conservative variant of SEQ ID NO: 34.
Embodiment 38. The method of any one of embodiments 35 to 37, wherein the anti-TSLP antibody or anti-TSLP antibody fragment is administered at a dose of 70 mg to 280 mg, e.g., 70 mg or 210 mg or 280 mg, at an interval of every 2 weeks, in particular every 4 weeks.
Embodiment 39. The method of any one of the preceding embodiments, wherein the antibody or antibody fragment is selected from the group consisting of a human antibody, humanized antibody, a chimeric antibody, a monoclonal antibody, a recombinant antibody, a human recombinant antibody.
Embodiment 40. The method of any one of the preceding embodiments, wherein the antibody fragment is selected from the group consisting of a Fab, Fab′, F(ab′)2, scFv, minibody, or diabody, in particular wherein the antibody fragment is a Fab, more particularly human or humanized Fab.
Embodiment 41. The method of embodiment 39, wherein the antibody is a human immunoglobulin.
Embodiment 42. The method according to any one of the preceding embodiments, further comprising administering a second agent to the subject.
Embodiment 43. The method of embodiment 42, wherein the second agent is selected from the group consisting of
   a) corticosteroids, e.g., inhaled corticosteroids (ICS) (e.g., fluticasone furoate, budesonide, beclometasone) or oral corticosteroids;
   b) bronchodilators, e.g., long-acting 32 agonist (LABA) (e.g., vilanterol, formoterol), short-acting 32 agonist (SABA) (e.g., salbuterol, levosalbuterol), anticholinergics, e.g., ipratropium, tiotropium, aclidinium and glycopyrronium;
   c) leukotriene receptor antagonists (LTRA), e.g., theophylline or its derivatives, Montekulast, Zafirlukast and Pranlukast;
   d) long-acting anti-muscarinics (LAMA), e.g., Tiotropium, umeclidinium, glycopyrronium;
   e) cromones;
   f) antihistamines;
   g) antileukotrienes; and
   h) PDE-4 inhibitors.
Embodiment 44. The method according to any one of the preceding embodiments, wherein the administration of the antibody or antibody fragment eliminates or reduces the need for the second agent therapy of embodiment 42 or 43, in particular for corticosteroid therapy.
Embodiment 45. An anti-TSLP antibody or anti-TSLP antibody fragment for use in the treatment of an inflammatory or obstructive airway disease, wherein the anti-TSLP antibody or anti-TSLP antibody fragment is administered to a subject at a dose of about 0.5 mg to about 16 mg, in particular about 2 mg to about 16 mg, more particularly about 4 mg to about 16 mg.
Embodiment 46. An anti-TSLP antibody or anti-TSLP antibody fragment for use in the treatment of asthma or reducing the frequency and/or severity of asthma exacerbations in a subject or reducing ACQ-5 score in a subjector treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the anti-TSLP antibody or anti-TSLP antibody fragment is administered to a subject at a dose of about 0.5 mg to about 16 mg, in particular about 2 mg to about 16 mg, more particularly about 4 mg to about 16 mg.
Embodiment 47. An anti-TSLP antibody or anti-TSLP antibody fragment for use in the treatment of asthma or reducing the frequency and/or severity of asthma exacerbations in a subject or reducing ACQ-5 score in a subjector treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a non-eosinophilic profile or a low eosinophil profile.
Embodiment 48. An anti-TSLP antibody or anti-TSLP antibody fragment for use in the treatment of asthma or reducing the frequency and/or severity of asthma exacerbations in a subject or reducing ACQ-5 score in a subjector treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a Th2 low profile.
Embodiment 49. The anti-TSLP antibody or anti-TSLP antibody fragment for use according to any one of embodiments 45 to 48, wherein the antibody or antibody fragment is administered at a dose of:
   a) about 0.5 mg, in particular of 0.5 mg; or
   b) about 1 mg, in particular of 1 mg; or
   c) about 2 mg, in particular of 2 mg; or
   d) about 4 mg, in particular of 4 mg; or
   e) about 8 mg, in particular of 8 mg; or
   f) about 16 mg, in particular 16 mg.
Embodiment 50. The anti-TSLP antibody or anti-TSLP antibody fragment for use according to any one of embodiments 45 to 49, wherein the dose is a daily dose.
Embodiment 51. The anti-TSLP antibody or anti-TSLP antibody fragment for use according to any one of embodiments 45 to 50, wherein the antibody or antibody fragment is administered at said dose once a day.
Embodiment 52. The anti-TSLP antibody or anti-TSLP antibody fragment for use according to any one of embodiments 45 to 51, wherein the antibody or antibody fragment is administered for a period of at least 1 month, at least 2 months, at least 3 months, at least 4 months, at least 6 months, at least 9 months, at least 1 year or more.
Embodiment 53. The anti-TSLP antibody or anti-TSLP antibody fragment for use according to any one of embodiments 45 to 52, wherein the antibody or antibody fragment is administered to the subject orally or intranasally.
Embodiment 54. The anti-TSLP antibody or anti-TSLP antibody fragment for use according to embodiment 53, wherein the antibody or antibody fragment is administered to the subject in an aerosolized form.
Embodiment 55. The anti-TSLP antibody or anti-TSLP antibody fragment for use according to embodiment 45 or 54, wherein the antibody or antibody fragment is administered to the subject by inhalation.
Embodiment 56. The anti-TSLP antibody or anti-TSLP antibody fragment for use according to embodiment 55, wherein the antibody or antibody fragment is administered to the subject by using a dry powder inhaler.
Embodiment 57. The anti-TSLP antibody or anti-TSLP antibody fragment for use according to any one of embodiment 45 to 56, wherein the subject has allergic asthma or non-allergic asthma.
Embodiment 58. The anti-TSLP antibody or anti-TSLP antibody fragment for use according to any one of embodiment 45 to 57, wherein the subject has mild asthma.
Embodiment 59. The anti-TSLP antibody or anti-TSLP antibody fragment for use according to any one of embodiment 45 to 57, wherein the subject has moderate asthma.
Embodiment 60. The anti-TSLP antibody or anti-TSLP antibody fragment for use according to any one of embodiment 45 to 57, wherein the subject has moderate-to-severe asthma or severe asthma.
Embodiment 61. The anti-TSLP antibody or anti-TSLP antibody fragment for use according to embodiment 59 or 60, wherein the subject has uncontrolled asthma, in particular severe uncontrolled asthma.
Embodiment 62. The anti-TSLP antibody or anti-TSLP antibody fragment for use according to any one of embodiments 45 to 61, wherein the subject has eosinophilic or non-eosinophilic asthma.
Embodiment 63. The anti-TSLP antibody or anti-TSLP antibody fragment for use according to any one of embodiments 45 to 61, wherein the subject has eosinophilic or non-eosinophilic COPD.
Embodiment 64. The anti-TSLP antibody or anti-TSLP antibody fragment for use according to embodiment 62 or 63, wherein the subject has high eosinophil count, e.g., >250 cells/μl, particularly >300 cells/μl, in particular at the time of start of treatment or at the time of diagnosis.
Embodiment 65. The anti-TSLP antibody or anti-TSLP antibody fragment for use according to embodiment 62 or 63, wherein the subject has low eosinophil count, e.g., <200 cells/μl, particularly <300 cells/μl, in particular at the time of start of treatment or at the time of diagnosis.
Embodiment 66. The anti-TSLP antibody or anti-TSLP antibody fragment for use according to any one of embodiments 45 to 65, wherein the subject has Th2 low profile.
Embodiment 67. The anti-TSLP antibody or anti-TSLP antibody fragment for use according embodiment 66, wherein the subject has has a Th2 profile of IgE less than or equal to 100 IU/ml or eosinophil count of less than 140 cells/μl, in particular at the time of start of treatment or at the time of diagnosis.
Embodiment 68. The anti-TSLP antibody or anti-TSLP antibody fragment for use according to any one of embodiments 45 to 67, wherein the subject is an adult or adolescent.
Embodiment 69. The anti-TSLP antibody or anti-TSLP antibody fragment for use according to any one of embodiments 45 to 67, wherein the subject is a child.
Embodiment 70. The anti-TSLP antibody or anti-TSLP antibody fragment for use according to any one of embodiments 45 to 69, wherein the subject is on background therapy of:
   (a) medium or high dose corticosteroids, e.g., inhaled corticosteroids (ICS); or
   (b) medium or high dose corticosteroids, e.g., inhaled corticosteroids (ICS), in combination with LABA (ICS/LABA); or
   (c) medium or high dose corticosteroids, e.g., inhaled corticosteroids (ICS), in combination with LABA and up to two additional to ICS-LABA controllers, e.g., LTRA, theophylline or its derivatives or LAMA, e.g., ICS/LABA/LAMA.
Embodiment 71. The anti-TSLP antibody or anti-TSLP antibody fragment for use according to any one of embodiments 45 to 70, wherein the subject is selected according to at least one of the following criteria:
   a) prior to treatment with the antibody or antibody fragment, the subject has a post-bronchodilator reversibility of FEV1>12% and >200 mL;
   b) prior to treatment with the antibody or antibody fragment, the subject has a spirometry with pre-bronchodilator FEV1>40% and <85%;
   c) prior to treatment with the antibody or antibody fragment, the subject has an ACQ-5 score >1.5.
Embodiment 72. The anti-TSLP antibody or anti-TSLP antibody fragment for use according to any one of embodiments 45 to 71, wherein said subject achieves by week 12, preferably by week 4 or week 8, of the treatment at least one of the following:
   a) an improvement in FVC;
   b) an improvement in the FEV1;
   c) an improvement of FeNO;
   d) an improvement in morning and evening peak expiratory flow rate (PEF);
   e) an improvement of one or more symptoms of asthma, e.g., a reduction of the daytime and nighttime asthma symptom score (ADSD and/or ANSD), as measured by an asthma symptom diary;
   f) a reduction in the ACQ-5 score; or
   g) a reduction of Asthma Quality of Life Questionnaire (AQLQ) score, e.g., AQLQ+12 score.
Embodiment 73. The anti-TSLP antibody or anti-TSLP antibody fragment for use according to any one of embodiments 45 to 72, wherein the administration decreases eosinophils in blood, sputum, broncheoalveolar fluid, or lungs of the subject.
Embodiment 74. The anti-TSLP antibody or anti-TSLP antibody fragment for use according to any one of embodiments 45 to 73, wherein the administration decreases Th2 cell count, in particular wherein the administration shifts cell counts in the subject from a Th2 high population to a Th2 low population.
Embodiment 75. The anti-TSLP antibody or anti-TSLP antibody fragment for use according to any one of embodiments 45 to 74, wherein the administration delays the time to an asthma exacerbation compared to a subject not receiving the anti-TSLP antibody or antibody fragment thereof.
Embodiment 76. The anti-TSLP antibody or anti-TSLP antibody fragment for use according to any one of embodiments 45 to 75, wherein the anti-TSLP antibody or anti-TSLP antibody fragment selected from any one of the following:
   a) an antibody or antibody fragment that comprises:
      a HCDR1 comprising the amino acid sequence of SEQ ID NO: 4;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3) comprising the amino acid sequence of SEQ ID NO: 13;
   b) an antibody or antibody fragment that comprises:
      a HCDR1 comprising the amino acid sequence of SEQ ID NO: 5;
      a HCDR2 comprising the amino acid sequence of SEQ ID NO: 6;
      a HCDR3 comprising the amino acid sequence of SEQ ID NO: 3;
      a LCDR1 comprising the amino acid sequence of SEQ ID NO: 14;
      a LCDR2 comprising the amino acid sequence of SEQ ID NO: 15; and
      a LCDR3 comprising the amino acid sequence of SEQ ID NO: 16; and
   c) an antibody or antibody fragment that comprises
      a HCDR1 comprising the amino acid sequence of SEQ ID NO: 1;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3 comprising the amino acid sequence of SEQ ID NO: 13.
Embodiment 77. The anti-TSLP antibody or anti-TSLP antibody fragment for use according to embodiment 76, wherein the anti-TSLP antibody or anti-TSLP antibody fragment comprises
   a) a heavy chain variable region comprising
      i. the amino acid sequence of SEQ ID NO: 7, or
      ii. a sequence at least 90% identical to SEQ ID NO: 7, or
      iii. a conservative variant of SEQ ID NO: 7; and
   b) a light chain variable region comprising
      i. the amino acid sequence of SEQ ID NO: 17, or
      ii. a sequence at least 90% identical to SEQ ID NO: 17, or
      iii. a conservative variant of SEQ ID NO: 17.
Embodiment 78. The anti-TSLP antibody or anti-TSLP antibody fragment for use according to embodiment 76 or 77, wherein the anti-TSLP antibody or anti-TSLP antibody fragment comprises
   a) a heavy chain comprising
      i. the amino acid sequence of SEQ ID NO: 9, or
      ii. a sequence at least 90% identical to SEQ ID NO: 9, or
      iii. a conservative variant of SEQ ID NO: 9, and
   b) a light chain comprising
      i. the amino acid sequence of SEQ ID NO: 19, or
      ii. a sequence at least 90% identical to SEQ ID NO: 19, or
      iii. a conservative variant of SEQ ID NO: 19.
Embodiment 79. The anti-TSLP antibody or anti-TSLP antibody fragment for use according to any of the embodiments 45 to 75, wherein the anti-TSLP antibody or anti-TSLP antibody fragment comprises: a HCDR1 comprising the amino acid sequence of SEQ ID NO: 28; a HCDR2 comprising the amino acid sequence of SEQ ID NO: 29; a HCDR3 comprising the amino acid sequence of SEQ ID NO: 30; a LCDR1 comprising the amino acid sequence of SEQ ID NO: 25; a LCDR2 comprising the amino acid sequence of SEQ ID NO: 26; and a LCDR3 comprising the amino acid sequence of SEQ ID NO: 27.
Embodiment 80. The anti-TSLP antibody or anti-TSLP antibody fragment for use according to embodiment 79, wherein the anti-TSLP antibody or anti-TSLP antibody fragment comprises:
   a) a heavy chain variable region comprising (or consisting of)
      i. the amino acid sequence of SEQ ID NO: 32, or
      ii. a sequence at least 90% identical to SEQ ID NO: 32, or
      iii. a conservative variant of SEQ ID NO: 32; and
   b) a light chain variable region comprising (or consisting of)
      i. the amino acid sequence of SEQ ID NO: 31, or
      ii. a sequence at least 90% identical to SEQ ID NO: 31, or
      iii. a conservative variant of SEQ ID NO: 31.
Embodiment 81. The anti-TSLP antibody or anti-TSLP antibody fragment for use according to embodiment 79 or embodiment 80, wherein the anti-TSLP antibody or anti-TSLP antibody fragment comprises:
   a) a heavy chain comprising (or consisting of)
      i. the amino acid sequence of SEQ ID NO: 33, or
      ii. a sequence at least 90% identical to SEQ ID NO: 33, or
      iii. a conservative variant of SEQ ID NO: 33, and
   b) a light chain comprising (or consisting of)
      i. the amino acid sequence of SEQ ID NO: 34, or
      ii. a sequence at least 90% identical to SEQ ID NO: 34, or
      iii. a conservative variant of SEQ ID NO: 34.
Embodiment 82. The anti-TSLP antibody or anti-TSLP antibody fragment for use according to any one of embodiments 79 to 81, wherein the anti-TSLP antibody or anti-TSLP antibody fragment is administered at a dose of 70 mg to 280 mg, e.g., 70 mg or 210 mg or 280 mg, at an interval of every 2 weeks, in particular every 4 weeks.
Embodiment 83. The anti-TSLP antibody or anti-TSLP antibody fragment for use according to any one of embodiments 45 to 82, wherein the antibody or antibody fragment is selected from the group consisting of a human antibody, humanized antibody, a chimeric antibody, a monoclonal antibody, a recombinant antibody.
Embodiment 84. The anti-TSLP antibody or anti-TSLP antibody fragment for use according to any one of embodiments 45 to 82, wherein the antibody fragment is selected from the group consisting of a Fab, Fab′, F(ab′)2, scFv, minibody, or diabody, in particular wherein the antibody fragment is a Fab, more particularly human or humanized Fab.
Embodiment 85. The anti-TSLP antibody or anti-TSLP antibody fragment for use according to embodiment 83, wherein the antibody is a human immunoglobulin.
Embodiment 86. The anti-TSLP antibody or anti-TSLP antibody fragment for use according to any one of embodiments 45 to 85, further comprising administering a second agent to the subject.
Embodiment 87. The anti-TSLP antibody or anti-TSLP antibody fragment for use according to embodiment 86, wherein the second agent is selected from the group consisting of
   a) corticosteroids, e.g., inhaled corticosteroids (ICS) (e.g., fluticasone furoate, budesonide, beclometasone) or oral corticosteroids;
   b) bronchodilators, e.g., long-acting 32 agonist (LABA) (e.g., vilanterol, formoterol), short-acting 32 agonist (SABA) (e.g., salbuterol, levosalbuterol), anticholinergics, e.g., ipratropium, tiotropium, aclidinium and glycopyrronium;
   c) leukotriene receptor antagonists (LTRA), e.g., theophylline or its derivatives, Montekulast, Zafirlukast and Pranlukast;
   d) long-acting anti-muscarinics (LAMA), e.g., Tiotropium, umeclidinium, glycopyrronium;
   e) cromones;
   f) antihistamines;
   g) antileukotrienes; and
   h) PDE-4 inhibitors.
Embodiment 88. The anti-TSLP antibody or anti-TSLP antibody fragment for use according to any one of embodiments 45 to 87, wherein the administration of the antibody or antibody fragment eliminates or reduces the need for the second agent therapy of embodiment 68 or 69, in particular for corticosteroid therapy.
Embodiment 89. Use of an anti-TSLP antibody or anti-TSLP antibody fragment for the manufacture of a medicament for treating of an inflammatory or obstructive airway disease, wherein the anti-TSLP antibody or anti-TSLP antibody fragment is administered to a subject at a dose of about 0.5 mg to about 16 mg, in particular about 2 mg to about 16 mg, more particularly about 4 mg to about 16 mg.
Embodiment 90. Use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject, wherein the anti-TSLP antibody or anti-TSLP antibody fragment is administered to a subject at a dose of about 0.5 mg to about 16 mg, in particular about 2 mg to about 16 mg, more particularly about 4 mg to about 16 mg.
Embodiment 91. Use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a non-eosinophilic profile or a low eosinophil profile.
Embodiment 92. Use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the antibody or anti-TSLP antibody fragment, wherein the subject has a Th2 low profile.
Embodiment 93. Use of an anti-TSLP antibody or anti-TSLP antibody fragment according to any one of embodiments 89 to 92, wherein the antibody or antibody fragment is administered at a dose of:
   a) about 0.5 mg, in particular of 0.5 mg; or
   b) about 1 mg, in particular of 1 mg; or
   c) about 2 mg, in particular of 2 mg; or
   d) about 4 mg, in particular of 4 mg; or
   e) about 8 mg, in particular of 8 mg; or
   f) about 16 mg, in particular 16 mg.
Embodiment 94. Use of an anti-TSLP antibody or anti-TSLP antibody fragment according to any one of embodiments 89 to 93, wherein the dose is a daily dose.
Embodiment 95. Use of an anti-TSLP antibody or anti-TSLP antibody fragment according to any one of embodiments 89 to 94, wherein the antibody or antibody fragment is administered at said dose once a day.
Embodiment 96. Use of an anti-TSLP antibody or anti-TSLP antibody fragment according to any one of embodiments 89 to 95, wherein the antibody or antibody fragment is administered for a period of at least 1 month, at least 2 months, at least 3 months, at least 4 months, at least 6 months, at least 9 months, at least 1 year or more.
Embodiment 97. Use of an anti-TSLP antibody or anti-TSLP antibody fragment according to any one of embodiments 89 to 96, wherein the antibody or antibody fragment is administered to the subject orally or intranasally.
Embodiment 98. Use of an anti-TSLP antibody or anti-TSLP antibody fragment according to embodiment 97, wherein the antibody or antibody fragment is administered to the subject in an aerosolized form.
Embodiment 99. Use of an anti-TSLP antibody or anti-TSLP antibody fragment according to embodiment 97 or 98, wherein the antibody or antibody fragment is administered to the subject by inhalation.
Embodiment 100. Use of an anti-TSLP antibody or anti-TSLP antibody fragment according to embodiment 99, wherein the antibody or antibody fragment is administered to the subject by using a dry powder inhaler.
Embodiment 101. Use of an anti-TSLP antibody or anti-TSLP antibody fragment according to any one of embodiments 89 to 100, wherein the subject has allergic asthma or non-allergic asthma.
Embodiment 102. Use of an anti-TSLP antibody or anti-TSLP antibody fragment according to any one of embodiments 89 to 101, wherein the subject has mild asthma.
Embodiment 103. Use of an anti-TSLP antibody or anti-TSLP antibody fragment according to any one of embodiments 89 to 101, wherein the subject has moderate asthma.
Embodiment 104. Use of an anti-TSLP antibody or anti-TSLP antibody fragment according to any one of embodiments 89 to 101, wherein the subject has moderate-to-severe asthma or severe asthma.
Embodiment 105. Use of an anti-TSLP antibody or anti-TSLP antibody fragment according to embodiment 103 or 104, wherein the subject has uncontrolled asthma, in particular severe uncontrolled asthma.
Embodiment 106. Use of an anti-TSLP antibody or anti-TSLP antibody fragment according to any one of embodiments 89 to 105, wherein the subject has eosinophilic or non-eosinophilic asthma.
Embodiment 107. Use of an anti-TSLP antibody or anti-TSLP antibody fragment according to any one of embodiments 89 to 105, wherein the subject has eosinophilic or non-eosinophilic COPD
Embodiment 108. Use of an anti-TSLP antibody or anti-TSLP antibody fragment according to embodiment 106 or 107, wherein the subject has high eosinophil count, e.g., ≥250 cells/μl, particularly >300 cells/μl, in particular at the time of start of treatment or at the time of diagnosis.
Embodiment 109. Use of an anti-TSLP antibody or anti-TSLP antibody fragment according to embodiment 106 or 107, wherein the subject has low eosinophil count, e.g., <200 cells/μl, particularly <300 cells/μl, in particular at the time of start of treatment or at the time of diagnosis.
Embodiment 110. Use of an anti-TSLP antibody or anti-TSLP antibody fragment according to any one of embodiments 89 to 109, wherein the subject has Th2 low profile.
Embodiment 111. Use of an anti-TSLP antibody or anti-TSLP antibody fragment according to embodiment 110, wherein the subject has has a Th2 profile of IgE less than or equal to 100 IU/ml or eosinophil count of less than 140 cells/μl, in particular at the time of start of treatment or at the time of diagnosis.
Embodiment 112. Use of an anti-TSLP antibody or anti-TSLP antibody fragment according to any one of embodiments 89 to 111, wherein the subject is an adult or adolescent.
Embodiment 113. Use of an anti-TSLP antibody or anti-TSLP antibody fragment according to any one of embodiments 89 to 111, wherein the subject is a child.
Embodiment 114. Use of an anti-TSLP antibody or anti-TSLP antibody fragment according to any one of embodiments 89 to 113, wherein the subject is on background therapy of:
   (a) medium or high dose corticosteroids, e.g., inhaled corticosteroids (ICS); or
   (b) medium or high dose corticosteroids, e.g., inhaled corticosteroids (ICS), in combination with LABA (ICS/LABA); or
   (c) medium or high dose corticosteroids, e.g., inhaled corticosteroids (ICS), in combination with LABA and up to two additional to ICS-LABA controllers, e.g., LTRA, theophylline or its derivatives or LAMA, e.g., ICS/LABA/LAMA.
Embodiment 115. Use of an anti-TSLP antibody or anti-TSLP antibody fragment according to any one of embodiments 89 to 114, wherein the subject is selected according to at least one of the following criteria:
   a) prior to treatment with the antibody or antibody fragment, the subject has a post-bronchodilator reversibility of FEV1>12% and >200 mL;
   b) prior to treatment with the antibody or antibody fragment, the subject has a spirometry with pre-bronchodilator FEV1>40% and <85%;
   c) prior to treatment with the antibody or antibody fragment, the subject has an ACQ-5 score >1.5.
Embodiment 116. Use of an anti-TSLP antibody or anti-TSLP antibody fragment according to any one of embodiments 89 to 115, wherein said subject achieves by week 12, preferably by week 4 or week 8, of the treatment at least one of the following:
   a) an improvement in FVC;
   b) an improvement in the FEV1;
   c) an improvement of FeNO;
   d) an improvement in morning and evening peak expiratory flow rate (PEF);
   e) an improvement of one or more symptoms of asthma, e.g., a reduction of the daytime and nighttime asthma symptom score (ADSD and/or ANSD), as measured by an asthma symptom diary;
   f) a reduction in the ACQ-5 score; or
   g) a reduction of Asthma Quality of Life Questionnaire (AQLQ) score, e.g., AQLQ+12 score.
Embodiment 117. Use of an anti-TSLP antibody or anti-TSLP antibody fragment according to any one of embodiments 89 to 116, wherein the administration decreases eosinophils in blood, sputum, broncheoalveolar fluid, or lungs of the subject.
Embodiment 118. Use of an anti-TSLP antibody or anti-TSLP antibody fragment according to any one of embodiments 89 to 117, wherein the administration decreases Th2 cell count, in particular wherein the administration shifts cell counts in the subject from a Th2 high population to a Th2 low population.
Embodiment 119. Use of an anti-TSLP antibody or anti-TSLP antibody fragment according to any one of embodiments 89 to 118, wherein the administration delays the time to an asthma exacerbation compared to a subject not receiving the anti-TSLP antibody or antibody fragment thereof.
Embodiment 120. Use of an anti-TSLP antibody or anti-TSLP antibody fragment according to any one of embodiments 89 to 119, wherein the anti-TSLP antibody or anti-TSLP antibody fragment selected from any one of the following:
   a) an antibody or antibody fragment that comprises:
      a HCDR1 comprising the amino acid sequence of SEQ ID NO: 4;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3) comprising the amino acid sequence of SEQ ID NO: 13;
   b) an antibody or antibody fragment that comprises:
      a HCDR1 comprising the amino acid sequence of SEQ ID NO: 5;
      a HCDR2 comprising the amino acid sequence of SEQ ID NO: 6;
      a HCDR3 comprising the amino acid sequence of SEQ ID NO: 3;
      a LCDR1 comprising the amino acid sequence of SEQ ID NO: 14;
      a LCDR2 comprising the amino acid sequence of SEQ ID NO: 15; and
      a LCDR3 comprising the amino acid sequence of SEQ ID NO: 16; and
   c) an antibody or antibody fragment that comprises
      a HCDR1 comprising the amino acid sequence of SEQ ID NO: 1;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3 comprising the amino acid sequence of SEQ ID NO: 13.
Embodiment 121. Use of an anti-TSLP antibody or anti-TSLP antibody fragment according to embodiment 120, wherein the anti-TSLP antibody or anti-TSLP antibody fragment comprises
   a) a heavy chain variable region comprising
      i. the amino acid sequence of SEQ ID NO: 7, or
      ii. a sequence at least 90% identical to SEQ ID NO: 7, or
      iii. a conservative variant of SEQ ID NO: 7; and
   b) a light chain variable region comprising
      i. the amino acid sequence of SEQ ID NO: 17, or
      ii. a sequence at least 90% identical to SEQ ID NO: 17, or
      iii. a conservative variant of SEQ ID NO: 17.
Embodiment 122. Use of an anti-TSLP antibody or anti-TSLP antibody fragment according to embodiment 120 or 121, wherein the anti-TSLP antibody or anti-TSLP antibody fragment comprises
   a) a heavy chain comprising
      i. the amino acid sequence of SEQ ID NO: 9, or
      ii. a sequence at least 90% identical to SEQ ID NO: 9, or
      iii. a conservative variant of SEQ ID NO: 9, and
   b) a light chain comprising
      i. the amino acid sequence of SEQ ID NO: 19, or
      ii. a sequence at least 90% identical to SEQ ID NO: 19, or
      iii. a conservative variant of SEQ ID NO: 19.
Embodiment 123. Use of an anti-TSLP antibody or anti-TSLP antibody fragment according to any one of embodiments 89 to 119, wherein the anti-TSLP antibody or anti-TSLP antibody fragment comprises: a HCDR1 comprising the amino acid sequence of SEQ ID NO: 28; a HCDR2 comprising the amino acid sequence of SEQ ID NO: 29; a HCDR3 comprising the amino acid sequence of SEQ ID NO: 30; a LCDR1 comprising the amino acid sequence of SEQ ID NO: 25; a LCDR2 comprising the amino acid sequence of SEQ ID NO: 26; and a LCDR3 comprising the amino acid sequence of SEQ ID NO: 27.
Embodiment 124. Use of an anti-TSLP antibody or anti-TSLP antibody fragment according to embodiment 123, wherein the anti-TSLP antibody or anti-TSLP antibody fragment comprises:
   a) a heavy chain variable region comprising (or consisting of)
      i. the amino acid sequence of SEQ ID NO: 32, or
      ii. a sequence at least 90% identical to SEQ ID NO: 32, or
      iii. a conservative variant of SEQ ID NO: 32; and
   b) a light chain variable region comprising (or consisting of)
      iv. the amino acid sequence of SEQ ID NO: 31, or
      v. a sequence at least 90% identical to SEQ ID NO: 31, or
      vi. a conservative variant of SEQ ID NO: 31.
Embodiment 125. Use of an anti-TSLP antibody or anti-TSLP antibody fragment according to embodiment 123 or embodiment 124, wherein the anti-TSLP antibody or anti-TSLP antibody fragment comprises:
   a) a heavy chain comprising (or consisting of)
      i. the amino acid sequence of SEQ ID NO: 33, or
      ii. a sequence at least 90% identical to SEQ ID NO: 33, or
      iii. a conservative variant of SEQ ID NO: 33, and
   b) a light chain comprising (or consisting of)
      i. the amino acid sequence of SEQ ID NO: 34, or
      ii. a sequence at least 90% identical to SEQ ID NO: 34, or
      iii. a conservative variant of SEQ ID NO: 34.
Embodiment 126. Use of an anti-TSLP antibody or anti-TSLP antibody fragment according to any one of embodiments 123 to 125, wherein the anti-TSLP antibody or anti-TSLP antibody fragment is administered at a dose of 70 mg to 280 mg, e.g., 70 mg or 210 mg or 280 mg, at an interval of every 2 weeks, in particular every 4 weeks.
Embodiment 127. Use of an anti-TSLP antibody or anti-TSLP antibody fragment according to any one of embodiments 89 to 126, wherein the antibody or antibody fragment is selected from the group consisting of a human antibody, humanized antibody, a chimeric antibody, a monoclonal antibody, a recombinant antibody.
Embodiment 128. Use of an anti-TSLP antibody or anti-TSLP antibody fragment according to any one of embodiments 89 to 127, wherein the antibody fragment is selected from the group consisting of a Fab, Fab′, F(ab′)2, scFv, minibody, or diabody, in particular wherein the antibody fragment is a Fab, more particularly human or humanized Fab.
Embodiment 129. Use of an anti-TSLP antibody or anti-TSLP antibody fragment according to embodiment 127, wherein the antibody is a human immunoglobulin.
Embodiment 130. Use of an anti-TSLP antibody or anti-TSLP antibody fragment according to any one of embodiments 89 to 129, further comprising administering a second agent to the subject.
Embodiment 131. Use of an anti-TSLP antibody or anti-TSLP antibody fragment according to embodiment 130, wherein the second agent is selected from the group consisting of
   a) corticosteroids, e.g., inhaled corticosteroids (ICS) (e.g., fluticasone furoate, budesonide, beclometasone) or oral corticosteroids;
   b) bronchodilators, e.g., long-acting 32 agonist (LABA) (e.g., vilanterol, formoterol), short-acting 32 agonist (SABA) (e.g., salbuterol, levosalbuterol), anticholinergics, e.g., ipratropium, tiotropium, aclidinium and glycopyrronium;
   c) leukotriene receptor antagonists (LTRA), e.g., theophylline or its derivatives, Montekulast, Zafirlukast and Pranlukast;
   d) long-acting anti-muscarinics (LAMA), e.g., Tiotropium, umeclidinium, glycopyrronium;
   e) cromones;
   f) antihistamines;
   g) antileukotrienes; and
   h) PDE-4 inhibitors.
Embodiment 132. Use of an anti-TSLP antibody or anti-TSLP antibody fragment according to any one of embodiments 89 to 131, wherein the administration of the antibody or antibody fragment eliminates or reduces the need for the second agent therapy of embodiment 103 or 104, in particular for corticosteroid therapy.
Embodiment 133. The method according to any one of embodiments 1 to 44, or the anti-TSLP antibody or anti-TSLP antibody fragment for use according to any one of embodiments 45 to 88, or use according to any one of embodiments 89 to 132, wherein the antibody or antibody fragment is disposed in a pharmaceutical composition, in particular wherein said pharmaceutical composition further comprises a pharmaceutically acceptable carrier.
Embodiment 134. The method according to embodiment 133, or the anti-TSLP antibody or anti-TSLP antibody fragment for use according to embodiment 133, or use according to embodiment 133, wherein the composition comprises 0.5 mg to 16 mg, in particular 2 mg to 16 mg, more particularly 4 mg to 16 mg, of the anti-TSLP antibody or anti-TSLP antibody fragment.
Embodiment 135. A pharmaceutical composition comprising spray-dried particles comprising:
   i. a core comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the composition, in particular about 3% to about 50% (w/w) of the composition;
      and
   ii. a shell comprising trileucine, wherein trileucine is about 1% to about 25% (w/w), in particular about 10% to about 15% (w/w) of the composition, preferably 10% w/w.
Embodiment 136. The pharmaceutical composition of embodiment 135, wherein the composition comprises 0.5 mg to 16 mg, in particular 2 mg to 16 mg, more particularly 4 mg to 16 mg, of the anti-TSLP antibody or anti-TSLP antibody fragment.
Embodiment 137. The pharmaceutical composition of embodiment 135 or 136, wherein the core further comprises trehalose.
Embodiment 138. The pharmaceutical composition of any one of embodiments 135 to 137, wherein the composition has a pH of 4.5 to 6.0.
Embodiment 139. The pharmaceutical composition of any one of embodiments 135 to 138, wherein the composition further comprises a buffer, e.g., histidine and/or HCl.
Embodiment 140. The pharmaceutical composition of any one of embodiments 135 to 139, wherein the anti-TSLP antibody or anti-TSLP antibody fragment is selected from any one of the following:
   a) an antibody or antibody fragment that comprises:
      a HCDR1 comprising the amino acid sequence of SEQ ID NO: 4;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3) comprising the amino acid sequence of SEQ ID NO: 13;
   b) an antibody or antibody fragment that comprises:
      a HCDR1 comprising the amino acid sequence of SEQ ID NO: 5;
      a HCDR2 comprising the amino acid sequence of SEQ ID NO: 6;
      a HCDR3 comprising the amino acid sequence of SEQ ID NO: 3;
      a LCDR1 comprising the amino acid sequence of SEQ ID NO: 14;
      a LCDR2 comprising the amino acid sequence of SEQ ID NO: 15; and
      a LCDR3 comprising the amino acid sequence of SEQ ID NO: 16; and
   c) an antibody or antibody fragment that comprises
      a HCDR1 comprising the amino acid sequence of SEQ ID NO: 1;
      a HCDR2 comprising the amino acid sequence of SEQ ID NO: 2;
      a HCDR3 comprising the amino acid sequence of SEQ ID NO: 3;
      a LCDR1 comprising the amino acid sequence of SEQ ID NO: 11;
      a LCDR2 comprising the amino acid sequence of SEQ ID NO: 12; and
      a LCDR3 comprising the amino acid sequence of SEQ ID NO: 13.
Embodiment 141. The pharmaceutical composition of embodiment 140, wherein the anti-TSLP antibody or anti-TSLP antibody fragment comprises
   a) a heavy chain variable region comprising
      i. the amino acid sequence of SEQ ID NO: 7, or
      ii. a sequence at least 90% identical to SEQ ID NO: 7, or
      iii. a conservative variant of SEQ ID NO: 7; and
   b) a light chain variable region comprising
      i. the amino acid sequence of SEQ ID NO: 17, or
      ii. a sequence at least 90% identical to SEQ ID NO: 17, or
      iii. a conservative variant of SEQ ID NO: 17.
Embodiment 142. The pharmaceutical composition of embodiment 140 or 141, wherein the anti-TSLP antibody or anti-TSLP antibody fragment comprises
   a) a heavy chain comprising
      i. the amino acid sequence of SEQ ID NO: 9, or
      ii. a sequence at least 90% identical to SEQ ID NO: 9, or
      iii. a conservative variant of SEQ ID NO: 9, and
   b) a light chain comprising
      i. the amino acid sequence of SEQ ID NO: 19, or
      ii. a sequence at least 90% identical to SEQ ID NO: 19, or
      iii. a conservative variant of SEQ ID NO: 19.
Embodiment 143. The pharmaceutical composition of any one of embodiments 135 to 142, wherein the antibody fragment is selected from the group consisting of a Fab, Fab′, F(ab′)2, scFv, minibody, or diabody, in particular wherein the antibody fragment is a Fab, more particularly human or humanized Fab.
Embodiment 144. The pharmaceutical composition of any one of embodiments 135 to 143, wherein the composition comprises about 3-3.5% (w/w), about 6-6.5% (w/w), about 12-13% (w/w), about 25% (w/w), about 50% (w/w) anti-TSLP antibody or anti-TSLP antibody fragment.
Embodiment 145. The pharmaceutical composition of any one of embodiments 135 to 144, wherein the composition comprises about 10% to about 95% (w/w) trehalose, in particular about 20% (w/w) to about 85% (w/w) trehalose.
Embodiment 146. The pharmaceutical composition of any one of embodiments 135 to 145, wherein trehalose: anti-TSLP antibody or anti-TSLP antibody fragment ratio is greater than 0.4, greater than 0.5, greater than 0.6, greater than 0.65, greater than 1, greater than 1.5, greater than 2, greater than 3, greater than 4, greater than 5, greater than 10, greater than 15, greater than 20, greater than 25.
Embodiment 147. The pharmaceutical composition of any one of embodiments 135 to 146, wherein the composition comprises about 2% (w/w) to about 15% (w/w) histidine, in particular about 5% (w/w) to about 9% (w/w) histidine.
Embodiment 148. The pharmaceutical composition of any one of embodiments 135 to 147, wherein the composition comprises about 0.5% (w/w) to about 4% (w/w) HCl, in particular about 1% (w/w) to about 2% (w/w) HCl.
Embodiment 149. The pharmaceutical composition of any one of embodiments 135 to 148, wherein the composition comprises:
   a) about 3% (w/w) anti-TSLP antibody or anti-TSLP antibody fragment, about 10% (w/w) trileucine, about 81% (w/w) trehalose, and a buffer;
   b) about 5% (w/w) anti-TSLP antibody or anti-TSLP antibody fragment, about 15% (w/w) trileucine, about 71% (w/w) trehalose, and a buffer;
   c) about 6% (w/w) anti-TSLP antibody or anti-TSLP antibody fragment, about 10% (w/w) trileucine, about 78% (w/w) trehalose, and a buffer;
   d) about 10% (w/w) anti-TSLP antibody or anti-TSLP antibody fragment, about 15% (w/w) trileucine, about 66% (w/w) trehalose, and a buffer;
   e) about 10% (w/w) anti-TSLP antibody or anti-TSLP antibody fragment, about 10% (w/w) trileucine, about 74% (w/w) trehalose, and a buffer;
   f) about 12.5% (w/w) anti-TSLP antibody or anti-TSLP antibody fragment, about 10% (w/w) trileucine, about 71% (w/w) trehalose, and a buffer;
   g) about 20% (w/w) anti-TSLP antibody or anti-TSLP antibody fragment, about 15% (w/w) trileucine, about 55.5% (w/w) trehalose, and a buffer;
   h) about 25% (w/w) anti-TSLP antibody or anti-TSLP antibody fragment, about 15% (w/w) trileucine, about 50% (w/w) trehalose, and a buffer;
   i) about 25% (w/w) anti-TSLP antibody or anti-TSLP antibody fragment, about 10% (w/w) trileucine, about 58% (w/w) trehalose, and a buffer;
   j) about 40% (w/w) anti-TSLP antibody or anti-TSLP antibody fragment, about 15% (w/w) trileucine, about 35% (w/w) trehalose, and a buffer;
   k) about 50% (w/w) anti-TSLP antibody or anti-TSLP antibody fragment, about 15% (w/w) trileucine, about 24.5% (w/w) trehalose, and a buffer;
   l) about 50% (w/w) anti-TSLP antibody or anti-TSLP antibody fragment, about 10% (w/w) trileucine, about 32.5% (w/w) trehalose, and a buffer.
Embodiment 150. The pharmaceutical composition of embodiment 149, wherein the composition comprises:
   a) about 3% (w/w) anti-TSLP antibody or anti-TSLP antibody fragment, about 10% (w/w) trileucine, about 81% (w/w) trehalose, about 5% (w/w) histidine, and about 1% (w/w) HCl;
   b) about 5% (w/w) anti-TSLP antibody or anti-TSLP antibody fragment, about 15% (w/w) trileucine, about 71% (w/w) trehalose, about 7.5% (w/w) histidine, and about 1.5% (w/w) HCl;
   c) about 6% (w/w) anti-TSLP antibody or anti-TSLP antibody fragment, about 10% (w/w) trileucine, about 78% (w/w) trehalose, about 5% (w/w) histidine, and about 1% (w/w) HCl;
   d) about 10% (w/w) anti-TSLP antibody or anti-TSLP antibody fragment, about 15% (w/w) trileucine, about 66% (w/w) trehalose, about 7.5% (w/w) histidine, and about 1.5% (w/w) HCl;
   e) about 10% (w/w) anti-TSLP antibody or anti-TSLP antibody fragment, about 10% (w/w) trileucine, about 74% (w/w) trehalose, about 5% (w/w) histidine, and about 1% (w/w) HCl;
   f) about 12.5% (w/w) anti-TSLP antibody or anti-TSLP antibody fragment, about 10% (w/w) trileucine, about 71% (w/w) trehalose, about 5% (w/w) histidine, and about 1% (w/w) HCl;
   g) about 20% (w/w) anti-TSLP antibody or anti-TSLP antibody fragment, about 15% (w/w) trileucine, about 55.5% (w/w) trehalose, about 8% (w/w) histidine, and about 1.5% (w/w) HCl;
   h) about 25% (w/w) anti-TSLP antibody or anti-TSLP antibody fragment, about 15% (w/w) trileucine, about 50% (w/w) trehalose, about 8% (w/w) histidine, and about 2% (w/w) HCl;
   i) about 25% (w/w) anti-TSLP antibody or anti-TSLP antibody fragment, about 10% (w/w) trileucine, about 58% (w/w) trehalose, about 6% (w/w) histidine, and about 1% (w/w) HCl;
   j) about 40% (w/w) anti-TSLP antibody or anti-TSLP antibody fragment, about 15% (w/w) trileucine, about 35% (w/w) trehalose, about 8% (w/w) histidine, and about 2% (w/w) HCl
   k) about 50% (w/w) anti-TSLP antibody or anti-TSLP antibody fragment, about 15% (w/w) trileucine, about 24.5% (w/w) trehalose, about 8.5% (w/w) histidine, and about 2% (w/w) HCl;
   l) about 50% (w/w) anti-TSLP antibody or anti-TSLP antibody fragment, about 10% (w/w) trileucine, about 32.5% (w/w) trehalose, about 6% (w/w) histidine, and about 1.5% (w/w) HCl.
Embodiment 151. The pharmaceutical composition of any one of embodiments 135 to 150, wherein the composition comprises:
   a) 3.13% (w/w) anti-TSLP antibody or anti-TSLP antibody fragment, 10% (w/w) trileucine, 80.83% (w/w) trehalose, 5.01% (w/w) histidine, and 1.04% (w/w) HCl;
   b) 5% (w/w) anti-TSLP antibody or anti-TSLP antibody fragment, 15% (w/w) trileucine, 70.97% (w/w) trehalose, 7.53% (w/w) histidine, and 1.56% (w/w) HCl;
   c) 6.25% (w/w) anti-TSLP antibody or anti-TSLP antibody fragment, 10% (w/w) trileucine, 77.61% (w/w) trehalose, 5.09% (w/w) histidine, and 1.05% (w/w) HCl;
   d) 10% (w/w) anti-TSLP antibody or anti-TSLP antibody fragment, 15% (w/w) trileucine, 65.76% (w/w) trehalose, 7.66% (w/w) histidine, and 1.59% (w/w) HCl;
   e) 10% (w/w) anti-TSLP antibody or anti-TSLP antibody fragment, 10% (w/w) trileucine, 73.73% (w/w) trehalose, 5.19% (w/w) histidine, and 1.08% (w/w) HCl;
   f) 12.5% (w/w) anti-TSLP antibody or anti-TSLP antibody fragment, 10% (w/w) trileucine, 71.15% (w/w) trehalose, 5.25% (w/w) histidine, and 1.09% (w/w) HCl;
   g) 20% (w/w) anti-TSLP antibody or anti-TSLP antibody fragment, 15% (w/w) trileucine, 55.44% (w/w) trehalose, 7.92% (w/w) histidine, and 1.64% (w/w) HCl;
   h) 25% (w/w) anti-TSLP antibody or anti-TSLP antibody fragment, 15% (w/w) trileucine, 50.29% (w/w) trehalose, 8.05% (w/w) histidine, and 1.67% (w/w) HCl;
   i) 25% (w/w) anti-TSLP antibody or anti-TSLP antibody fragment, 10% (w/w) trileucine, 58.26% (w/w) trehalose, 5.58% (w/w) histidine, and 1.16% (w/w) HCl;
   j) 40% (w/w) anti-TSLP antibody or anti-TSLP antibody fragment, 15% (w/w) trileucine, 34.81% (w/w) trehalose, 8.43% (w/w) histidine, and 1.75% (w/w) HCl
   k) 50% (w/w) anti-TSLP antibody or anti-TSLP antibody fragment, 15% (w/w) trileucine, 24.5% (w/w) trehalose, 8.69% (w/w) histidine, and 1.81% (w/w) HCl
   l) 50% (w/w) anti-TSLP antibody or anti-TSLP antibody fragment, 10% (w/w) trileucine, 32.48% (w/w) trehalose, 6.22% (w/w) histidine, and 1.3% (w/w) HCl.
Embodiment 152. The pharmaceutical composition of any one of embodiments 135 to 151, wherein said composition is used in the preparation of a medicament, in particular a capsule or a blister or a blister package, which is suitable for administration through inhalation, more particularly wherein each of the capsules or blister comprises from 0.5 mg to 16 mg, in particular from 2 mg to 16 mg, more particularly from 4 mg to 16 mg, of the anti-TSLP antibody or anti-TSLP antibody fragment.
Embodiment 153. A medicament for administration through inhalation comprising an anti-TSLP antibody or anti-TSLP antibody fragment, in particular an anti-TSLP antibody fragment, more particularly an anti-TSLP antibody Fab, wherein the medicament comprises 0.5 mg to 16 mg, in particular 2 mg to 16 mg, more particularly 4 mg to 16 mg, of said anti-TSLP antibody or anti-TSLP antibody fragment.
Embodiment 154. The medicament of embodiment 153, wherein the medicament comprises the pharmaceutical composition of any one of embodiments 135 to 152.
Embodiment 155. The medicament according to any one of embodiments 153 to 154, wherein the medicament is in a powder form, in particular in a dry powder form.
Embodiment 156. The medicament according to any one of embodiments 153 to 155, wherein the medicament is in powder form stored in a capsule or in a blister, in particular wherein said capsule or said blister is suitable for administration via inhalation.
Embodiment 157. A kit comprising the pharmaceutical composition of any one of embodiments 108 to 125 or the medicament of any one of embodiments 153 to 156, and a device for delivering the pharmaceutical composition or the medicament to a subject.
Embodiment 158. The kit of embodiment 157, wherein the device delivers the pharmaceutical composition or the medicament in an aerosolized form.
Embodiment 159. The kit of embodiment 157 or 158, wherein the device is a dry powder inhaler.
Embodiment 160. The pharmaceutical composition of any one of embodiments 135 to 152 or the medicament of any one of embodiments 153 to 156 for use in the treatment of an inflammatory or obstructive airway disease.
Embodiment 161. The pharmaceutical composition of embodiment 160 or the medicament of embodiment 160 for use in the treatment of an inflammatory or obstructive airway disease according to any one of embodiments 1 to 44.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 Arithmetic mean serum CSJ117 concentration-time profiles following single dose inhaled administration of CSJ117 (liner view). CSJ117 concentrations were measured up to 672 h post dose. For clarity, profiles are depicted up to 96 h post dose.

FIG. 2 Arithmetic mean serum CSJ117 concentration-time profiles following single dose inhaled administration of CSJ117 (semi-logarithmic view). CSJ117 concentrations were measured up to 672 h post dose. For clarity, profiles are depicted up to 96 h post dose.

FIG. 3 Study design of a randomized, subject- and investigator-blinded, placebo-controlled, parallel-design, bronchoprovocation study to evaluate the safety, tolerability, pharmacokinetics and pharmacodynamics of multiple doses of inhaled CSJ117 in adult subjects with mild atopic asthma. Twelve week dosing period with 3 allergen inhalation challenges (AIC): screening, Day 42 and Day 84.

FIG. 4 Arithmetic mean (+/−SE) of % change from pre-AIC FEV1 (Pharmacodynamic analysis set). Allergen inhalation challenge on Day 42 and Day 84.

FIG. 5 Arithmetic mean (+/−SE) of change from baseline of fractional exhaled nitric oxide (FeNO). Allergen inhalation challenge on Day 42 and Day 84.

FIG. 6 Linear plot of CSJ117 dose vs % Effect.

FIG. 7 Semi-log plot of CSJ117 dose vs % Effect.

FIG. 8 Difference (%) in fine particle fraction of formulation 13 (1, 4, 16 mg) compared to reference Formulation 13 (4 mg) and measured by next generation impactor (NGI).

FIG. 9 Difference (%) in fine particle fraction of formulation 1 (0.5 mg), 11 (16 mg) and 12 (16 mg) compared to reference Formulation 8 (4 mg) and measured by next generation impactor (NGI).

FIG. 10 Percent (%) change in aggregates over 6 months storage stability for new anti-TSLP Fab1 formulations (formulation 1, 8, 11, 12) as measured by size exclusion chromatography.

FIG. 11 Percent (%) change in fine particle fraction over 6 months storage stability for Formulations 1, 8, 11 and 12 measured by next generation impactor.

### DETAILED DESCRIPTION

The present invention is based on the surprising finding that an anti-TSLP antibody or anti-TSLP antibody fragment, when provided at a specific concentration and/or at specific time intervals and/or by a specific route of administration, e.g., inhalation, has an exceptional ability to reduce or prevent an inflammatory or obstructive airway disease in a subject. Accordingly, an anti-TSLP antibody or anti-TSLP antibody fragment, when provided at a specific concentration and/or at specific time intervals and/or by a specific route of administration as disclosed herein, is useful in the treatment of inflammatory or obstructive airway diseases, resulting, e.g., in reduction of tissue damage, airway inflammation, bronchial hyperreactivity, remodelling or disease progression. The treatment described herein provides a long needed improvement in the treatment of patients with an inflammatory or obstructive airway disease. Furthermore, as demonstrated herein, it can be used, and shows benefit, in combination with concomitant immunosuppressive agent, such as cortocosteroid, treatment, if desired.

The present invention is based on the finding that treatment of an inflammatory or obstructive airway disease comprising administering to a subject in need thereof a dose of about 0.5 mg to about 16 mg, in particular about 2 mg to about 16 mg, more particularly about 4 mg to about 16 mg, of an anti-TSLP antibody or anti-TSLP antibody fragment is particularly effective. Specifically, the inventors have found that treatment of an inflammatory or obstructive airway disease comprising administering to a subject in need thereof a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment, more particularly wherein said dose is administered by inhalation, is particularly effective. More particularly, the inventors have found that treatment of an inflammatory or obstructive airway disease comprising administering to a subject in need thereof a daily dose of about 0.5 mg to about 16 mg, e.g., a daily dose of about 1 mg to about 16 mg, a daily dose of about 2 mg to about 16 mg, a daily dose of about 2 mg to about 8 mg, in particular a daily dose of about 4 mg to about 8 mg, of an anti-TSLP antibody or anti-TSLP antibody fragment, more particularly wherein said dose is administered by inhalation, is particularly effective.

Certain terms used herein are described below. Unless defined otherwise, all technical and scientific terms used herein have the same meaning as is commonly understood by one of skill in the art to which this invention belongs. The following general definitions shall apply in this specification, unless otherwise specified:

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 20%, preferably up to 10%, more preferably up to 5%, from the stated amount: this usage of “about” recognizes that the precise amount in a given dosage form may differ slightly from an intended amount for various reasons without materially affecting the in vivo effect of the administered compound. When describing a composition herein as “about” a specified amount, the actual amount can vary by up to 20%, preferably up to 10%, more preferably up to 5%, from the stated amount: this usage of “about” recognizes that the precise amount in a given composition may differ slightly from an intended amount for various reasons without materially affecting the physiochemical properties and in vivo effect of the composition.

The terms “comprising” and “including” are used herein in their open-ended and non-limiting sense unless otherwise noted. As used herein, the term “comprising” encompasses “including” as well as “consisting”, e.g. a composition “comprising” X may consist exclusively of X or may include something additional, e.g., X+Y.

The term “treat”, “treating” or “treatment” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors. The invention relates to uses or methods of treatment of inflammatory or obstructive airway disease, e.g., asthma, COPD, wherein the treatment comprises treating or alleviating one or more symptoms of said inflammatory or obstructive airway disease, e.g., asthma, COPD, such as reducing the frequency and/or severity of asthma or COPD exacerbations, reducing ACQ-score in a subject, improving FVC, improving FEV1, improving FeNO, improving morning and evening peak expiratory flow rate (PEF), reducing the daytime and nighttime asthma symptom score (ADSD and/or ANSD), as measured by an asthma symptom diary, reducing Asthma Quality of Life Questionnaire (AQLQ) score, e.g., AQLQ+12 score, reducing EXACT—Respiratory Symptoms (E-RS) score, reducing COPD Assessment Test (CAT) score, and/or a reducing St. George's Respiratory Questionnaire (SGRQ) Score, reducing mMRC (Modified Medical Research Council) Dyspnea score. Thus, as used herein, the terms “treat”, “treatment” and “treating” refer to the reduction or amelioration of the progression, severity and/or duration of an inflammatory or obstructive airway disease, e.g., asthma, COPD, or the amelioration of one or more symptoms, suitably of one or more discernible symptoms, of an inflammatory or obstructive airway disease, e.g., asthma, COPD, resulting from the administration of an anti-TSLP antibody or anti-TSLP antibody fragment, when provided at a specific concentration and/or at specific time intervals and/or by a specific route of administration as disclosed herein. In specific embodiments, the terms “treat”, “treatment” and “treating” refer to the amelioration of at least one measurable physical parameter of an inflammatory or obstructive airway disease, e.g., asthma, COPD, wherein the physical parameter is not necessarily discernible by the patient.

The term “treatment” or “treat” includes treating a patient suspected to have the disease as well as patients who are ill or who have been diagnosed as suffering from the disease or medical condition, and includes suppression of clinical relapse.

### Treatment of Inflammatory or Obstructive Airway Diseases

In one aspect, the present invention provides a method of treating an inflammatory or obstructive airway disease comprising administering to a subject in need thereof a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment.

In one aspect, the present invention provides a method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, comprising administering to a subject in need thereof a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment. In one aspect, the present invention provides a method of improving one or more asthma symptoms, e.g., a method of improving FVC and/or FEV1 and/or FeNO and/or morning and evening peak expiratory flow rate (PEF) or reducing of the daytime and/or nighttime asthma symptom score (ADSD and/or ANSD), as measured by an asthma symptom diary, and/or reducing ACQ-5 score and/or reducing Asthma Quality of Life Questionnaire (AQLQ) score, e.g., AQLQ+12 score, and/or reducing rate of and severity of exacerbations. In one aspect, the present invention provides a method of improving one or more COPD symptoms, e.g., a method of improving FVC and/or FEV1 and/or reducing EXACT—Respiratory Symptoms (E-RS) score and/or reducing COPD Assessment Test (CAT) score and/or reducing St. George's Respiratory Questionnaire (SGRQ) score and/or reducing mMRC (Modified Medical Research Council) Dyspnea score and/or reducing rate of and severity of exacerbations.

In one aspect, the present invention provides a method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or anti-TSLP antibody fragment, wherein the subject has a non-eosinophilic profile or a low eosinophil profile.

In one aspect, the present invention provides a method for treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or antibody variant, wherein the antibody or anti-TSLP antibody fragment, wherein the subject has a Th2 low profile.

In one aspect, the present invention provides an anti-TSLP antibody or anti-TSLP antibody fragment for use in the treatment of an inflammatory or obstructive airway disease, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment.

In one aspect, the present invention provides an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg. In one aspect, the present invention provides an anti-TSLP antibody or anti-TSLP antibody fragment for use in improving one or more asthma symptoms, e.g., improving FVC and/or FEV1 and/or FeNO and/or morning and evening peak expiratory flow rate (PEF) or reducing of the daytime and/or nighttime asthma symptom score (ADSD and/or ANSD), as measured by an asthma symptom diary, and/or reducing ACQ-5 score and/or reducing Asthma Quality of Life Questionnaire (AQLQ) score, e.g., AQLQ+12 score, and/or reducing rate of and severity of exacerbations. In one aspect, the present invention provides an anti-TSLP antibody or anti-TSLP antibody fragment for use in improving one or more COPD symptoms, e.g., improving FVC and/or FEV1 and/or reducing EXACT—Respiratory Symptoms (E-RS) score and/or reducing COPD Assessment Test (CAT) score and/or reducing St. George's Respiratory Questionnaire (SGRQ) score and/or reducing mMRC (Modified Medical Research Council) Dyspnea score and/or reducing rate of and severity of exacerbations.

In one aspect, the present invention provides an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, wherein the subject has a non-eosinophilic profile or a low eosinophil profile.

In one aspect, the present invention provides an anti-TSLP antibody or anti-TSLP antibody fragment for use in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score or improving asthma or COPD symptoms in a subject in a subject, wherein the subject has a Th2 low profile.

In one aspect the present invention provides, use of an anti-TSLP antibody or anti-TSLP antibody fragment in the treatment of an inflammatory or obstructive airway disease, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment.

In one aspect the present invention provides, use of an anti-TSLP antibody or anti-TSLP antibody fragment in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment. In one aspect, the present invention provides use of an anti-TSLP antibody or anti-TSLP antibody fragment in improving one or more asthma symptoms, e.g., improving FVC and/or FEV1 and/or FeNO and/or morning and evening peak expiratory flow rate (PEF) or reducing of the daytime and/or nighttime asthma symptom score (ADSD and/or ANSD), as measured by an asthma symptom diary, and/or reducing ACQ-5 score and/or reducing Asthma Quality of Life Questionnaire (AQLQ) score, e.g., AQLQ+12 score, and/or reducing rate of and severity of exacerbations. In one aspect, the present invention provides use of an anti-TSLP antibody or anti-TSLP antibody fragment in improving one or more COPD symptoms, e.g., improving FVC and/or FEV1 and/or reducing EXACT—Respiratory Symptoms (E-RS) score and/or reducing COPD Assessment Test (CAT) score and/or reducing St. George's Respiratory Questionnaire (SGRQ) score and/or reducing mMRC (Modified Medical Research Council) Dyspnea score and/or reducing rate of and severity of exacerbations.

In one aspect the present invention provides, use of an anti-TSLP antibody or anti-TSLP antibody fragment in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or anti-TSLP antibody fragment, wherein the subject has a non-eosinophilic profile or a low eosinophil profile.

In one aspect the present invention provides, use of an anti-TSLP antibody or anti-TSLP antibody fragment in the treatment of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject comprising administering a therapeutically effective amount of an anti-TSLP antibody or anti-TSLP antibody fragment, wherein the subject has a Th2 low profile

In one aspect, the present invention provides use of an anti-TSLP antibody or anti-TSLP antibody fragment for the manufacture of a medicament for treating of an inflammatory or obstructive airway disease, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg of an anti-TSLP antibody or anti-TSLP antibody fragment.

In one aspect the present invention provides, use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms, wherein the anti-TSLP antibody or anti-TSLP antibody fragment is administered to a subject at a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment. In one aspect, the present invention provides use of an anti-TSLP antibody or anti-TSLP antibody fragment for the manufacture of a medicament for improving one or more asthma symptoms, e.g., improving FVC and/or FEV1 and/or FeNO and/or morning and evening peak expiratory flow rate (PEF) or reducing of the daytime and/or nighttime asthma symptom score (ADSD and/or ANSD), as measured by an asthma symptom diary, and/or reducing ACQ-5 score and/or reducing Asthma Quality of Life Questionnaire (AQLQ) score, e.g., AQLQ+12 score, and/or reducing rate of and severity of exacerbations. In one aspect, the present invention provides use of an anti-TSLP antibody or anti-TSLP antibody fragment for the manufacture of a medicament for improving one or more COPD symptoms, e.g., improving FVC and/or FEV1 and/or reducing EXACT—Respiratory Symptoms (E-RS) score and/or reducing COPD Assessment Test (CAT) score and/or reducing St. George's Respiratory Questionnaire (SGRQ) score and/or reducing mMRC (Modified Medical Research Council) Dyspnea score and/or reducing rate of and severity of exacerbations.

In one aspect the present invention provides, use of an anti-TSLP antibody or anti-TSLP antibody fragment for the manufacture of a medicament for treating of asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms In a subject, wherein the subject has a non-eosinophilic profile or a low eosinophil profile and/or wherein the subject has a Th2 low profile.

In one embodiment, the antibody or antibody fragment is administered to a subject at a dose of about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg or about 16 mg.

In one embodiment, said dose is administered upon need. Suitably, an anti-TSLP antibody or anti-TSLP antibody fragment is administered at a dose and frequency which is sufficient to reduce and/or ameliorate the severity and/or duration of a given condition, disorder, or disease and/or a symptom related thereto. These terms also encompass an amount and frequency necessary for the reduction, slowing, or amelioration of the advancement or progression of a given condition, disorder, or disease, reduction, slowing, or amelioration of the recurrence, development or onset of a given condition, disorder or disease, and/or to improve or enhance the prophylactic or therapeutic effect(s) of another therapy. Suitably, an anti-TSLP antibody or anti-TSLP antibody fragment is administered at a dose and frequency which is needed to achieve a specified result, for example, improvements in asthma- or COPD-associated parameters, such as reducing the frequency and/or severity of asthma or COPD exacerbations, reducing ACQ-5 score in a subject, improving FVC, improving FEV1, improving FeNO, improving morning and evening peak expiratory flow rate (PEF), reducing the daytime and nighttime asthma symptom score (ADSD and/or ANSD), as measured by an asthma symptom diary, reducing Asthma Quality of Life Questionnaire (AQLQ) score, e.g., AQLQ+12 score, reducing EXACT—Respiratory Symptoms (E-RS) score and/or reducing COPD Assessment Test (CAT) score and/or reducing St. George's Respiratory Questionnaire (SGRQ) score and/or reducing mMRC (Modified Medical Research Council) Dyspnea score. In one embodiment, the antibody or antibody fragment is administered to a subject at a dose of 16 mg or 8 mg or 4 mg or 2 mg and said dose is administered upon need. In one embodiment, the antibody or antibody fragment is administered to a subject at a dose which does not exceed 16 mg or does not exceed 8 mg or does not exceed 4 mg or does not exceed 2 mg, and said dose is administered upon need. In one embodiment, the antibody or antibody fragment is administered at said dose once a day. Suitably, said dose is a daily dose. In another embodiment, the antibody or antibody fragment is administered at said dose once a week. Suitably, said dose is a weekly dose.

In one embodiment, the anti-TSLP antibody or anti-TSLP antibody fragment is administered to a subject at a dose of 0.5 mg to 16 mg, e.g., 1 mg to 16 mg, 2 mg to 16 mg, 2 mg to 8 mg, in particular a dose of 4 mg to 8 mg. In one embodiment, the anti-TSLP antibody or anti-TSLP antibody fragment is administered to a subject at a dose of about 0.5 mg, in particular 0.5 mg; or about 1 mg, in particular 1 mg; or about 2 mg, in particular 2 mg; or about 3 mg, in particular 3 mg; or about 4 mg, in particular 4 mg; or about 5 mg, in particular 5 mg; or about 6 mg, in particular 6 mg; or about 7 mg, in particular 7 mg; or about 8 mg, in particular 8 mg; or about 9 mg, in particular 9 mg; or about 10 mg, in particular 10 mg; or about 11 mg, in particular 11 mg; or about 12 mg, in particular 12 mg; or about 13 mg, in particular 13 mg; or about 14 mg, in particular 14 mg; or about 15 mg, in particular 15 mg; or about 16 mg, in particular 16 mg. In one embodiment, the anti-TSLP antibody or anti-TSLP antibody fragment is administered to a subject at a dose of 0.5 mg to 16 mg, e.g., 1 mg to 16 mg, 2 mg to 16 mg, 2 mg to 8 mg, in particular a dose of 4 mg to 8 mg. In one embodiment, the anti-TSLP antibody or anti-TSLP antibody fragment is administered to a subject at a dose of: (a) about 0.5 mg, in particular 0.5 mg; or (b) about 1 mg, in particular 1 mg; or (c) about 2 mg, in particular 2 mg; or (d) about 4 mg, in particular 4 mg; or (e) about 8 mg, in particular 8 mg; or (f) about 16 mg, in particular 16 mg.

In one embodiment, said dose is administered upon need. In one embodiment, said dose is administered upon need, and the dose does not exceed 16 mg, in particular does not exceed 8 mg, more particularly does not exceed 4 mg. In a more specific embodiment, said dose is a daily dose. In a preferred embodiment, the antibody or antibody fragment is administered at said dose once a day. In a further embodiment, said dose is a weekly dose. In a further embodiment, the antibody or antibody fragment is administered at said dose once a week.

In one embodiment, the anti-TSLP antibody or anti-TSLP antibody fragment is administered to a subject for a period of at least 1 month, at least 2 months, at least 3 months, at least 4 months, at least 6 months, at least 9 months, at least 1 year or more. In one embodiment, the anti-TSLP antibody or anti-TSLP antibody fragment is administered to a subject for a period of at least 1 week, at least 2 weeks, at least 3 weeks, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 52 weeks or more of treatment. In one embodiment, the anti-TSLP antibody or anti-TSLP antibody fragment is administered to a subject until one or more symptoms of said inflammatory or obstructive airway disease, e.g., asthma, COPD, are treated or alleviated, e.g., reduction of the frequency and/or severity of asthma or COPD exacerbations, reduction of ACQ-5 score in a subject, an improvement in FVC, an improvement in the FEV1, an improvement of FeNO, an improvement in morning and evening peak expiratory flow rate (PEF), a reduction of the daytime and nighttime asthma symptom score (ADSD and/or ANSD), as measured by an asthma symptom diary, a reduction of Asthma Quality of Life Questionnaire (AQLQ) score, e.g., AQLQ+12 score, a reduction of EXACT—Respiratory Symptoms (E-RS) score, a reduction of COPD Assessment Test (CAT) score, and/or a reduction of St. George's Respiratory Questionnaire (SGRQ) Score and/or a reduction of mMRC (Modified Medical Research Council) Dyspnea score.

In some embodiments, the anti-TSLP antibody or anti-TSLP antibody fragment is formulated as a dry powder formulation suitable for inhalation. In some embodiments, the anti-TSLP antibody or anti-TSLP antibody fragment is administered to a subject orally or intranasally, e.g., in an aerosolized form. In some specific embodiments, the anti-TSLP antibody or anti-TSLP antibody fragment is administered to a subject by inhalation, in particular wherein the anti-TSLP antibody or anti-TSLP antibody fragment is administered to the subject by using a dry powder inhaler.

### Inflammatory or Obstructive Airway Diseases

Inflammatory or obstructive airway diseases to which the present invention is applicable include asthma of whatever type or genesis including both intrinsic (non-allergic) asthma and extrinsic (allergic) asthma, mild asthma, moderate asthma, severe asthma, moderate-to-severe asthma, bronchitis asthma, exercise-induced asthma, occupational asthma and asthma induced following bacterial infection or viral infection. In one aspect, the present invention provides a method of treating asthma or reducing the frequency and or severity of asthma exacerbations or improving asthma symptoms or reducing ACQ-5 score in a subject.

As used herein, the term “asthma” refers to a common and potentially serious chronic disease of the respiratory tract characterized by airway inflammation and constriction leading to symptoms such as wheezing, shortness of breath, chest tightness and cough that vary over time in their occurrence, frequency, intensity and response to therapy. The term “asthma” as used herein refers to asthma of all phenotypes and endotypes in all severity levels regardless the disease control status, such as non-allergic asthma, allergic asthma, mild asthma, moderate asthma, severe asthma, moderate-to-severe asthma, uncontrolled asthma, non-eosinophilic asthma, low eosinophil asthma, high eosinophil (eosinophilic) asthma, bronchitis asthma, exercise-induced asthma, occupational asthma and asthma induced following bacterial or viral infection.

The term “asthma exacerbation” refers to an acute or sub-acute worsening in asthma symptoms and lung function from the patient's usual status that can lead to any of the following: Use of systemic corticosteroids for at least 3 days; a single depo-injectable dose of corticosteroids is considered equivalent to a 3-day course of systemic corticosteroids; for subjects receiving maintenance OCS, a temporary doubling of the maintenance dose for at least 3 days qualifies; an ED visit due to asthma that required systemic corticosteroids (as per above); an inpatient hospitalization due to asthma. Additional measures associated with asthma exacerbations are also being examined to determine effect. These include hospitalizations related to asthma exacerbations (i.e., severe asthma exacerbations), time to first asthma exacerbation, and the proportion of subjects with one or more asthma exacerbation/severe asthma exacerbation. Severe exacerbations of asthma are usually defined as a requirement for systemic corticosteroids or an increase of the maintenance dose of oral corticosteroids for at least three days and/or a need for an emergency visit, hospitalization or death due to asthma. Moderate exacerbations are usually defined as events that require a change in treatment to avoid progression of worsening asthma to a severe exacerbation and the occurrence of one or more of the following—deterioration of symptoms of asthma, increased use of reliever medication, deterioration in lung function, which last for two days or more but not severe enough to warrant systemic corticosteroids or hospitalization.

Suitably, the methods, antibodies for uses, uses, compositions, medicaments and kits of the present invention are useful in treatment of allergic asthma or non-allergic asthma. In particular, the methods, antibodies for uses, uses, compositions, medicaments and kits of the present invention are useful in treatment of allergic asthma.

The term “allergic asthma” refers to asthma that is triggered by one or more inhaled allergens. Such patients have a positive IgE fluorescence enzyme immunoassay (FEIA) level to one or more allergens that trigger an asthmatic response. Typically, most allergic asthma is associated with Th2-type inflammation.

The term “Th2-type inflammation” as used herein refers to a subject having a screening blood eosinophil count >140 cells/μl and a screening total serum IgE level of >100 IU/mL (Corren et al, N Engl J Med. 22; 365(f 2):1088-98, 2011). A “Th2 high” asthma or COPD population or profile refers to a subject having IgE>100 IU/mL and Blood Eosinophil Count >140 cells/μl. A “Th2 low” asthma or COPD population refers to a subject having IgE<100 IU/mL and Blood Eosinophil Count <140 cells/μl.

The term “IgE” refers to immunoglobulin E, well known to a person skilled in the art. IgE is measured in international units per milliliter (IU/mL) as disclosed, e.g., in Seagroatt and Anderson (1981).

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

An eosinophil count is a blood test that measures the number of white blood cells called eosinophils. Blood is drawn from a vein, typically on the inside of the elbow or the back of the hand or using a tool such as a lancet to prick the skin. The blood is put in a small glass tube, or onto a slide or test strip. In a lab, the blood is placed on a microscope slide, and a stain is added to the sample. This causes eosinophils to show up as orange-red granules which make it possible to count how many eosinophils are present in a specific volume of blood, such as one microliter (L).

Suitably, the methods, antibodies for uses, uses, compositions, medicaments and kits of the present invention are useful in treatment of eosinophilic or non-eosinophilic asthma. In particular, the methods, antibodies for uses, uses, compositions, medicaments and kits of the present invention are useful in treatment of eosinophilic asthma. The term “non-eosinophilic asthma” refers to an asthma, wherein a patient has no eosinophil count. The term “low eosinophil asthma” refers to refers to an asthma, wherein a patient has a screening blood eosinophil count of <300 cells/μl, in particular at the time of start of treatment or at the time of diagnosis. The term “high eosinophil asthma” or “eosinophilic asthma” refers to an asthma, wherein a patient has a screening blood eosinophil count of >300 cells/μl, in particular at the time of start of treatment or at the time of diagnosis. Suitably, the methods, antibodies for uses, uses, compositions, medicaments and kits of the present invention are useful in treatment of asthma in a patient with low Th2 profile, e.g., wherein a patient has IgE<100 IU/mL and Blood Eosinophil Count <140 cells/μl, in particular at the time of start of treatment or at the time of diagnosis. Suitably, the methods, antibodies for uses, uses, compositions, medicaments and kits of the present invention are useful in treatment of asthma in a patient with high Th2 profile, e.g., wherein a patient has IgE>100 IU/mL and Blood Eosinophil Count >140 cells/μl, in particular at the time of start of treatment or at the time of diagnosis.

Suitably, the methods, antibodies for uses, uses, compositions, medicaments and kits of the present invention are useful in treatment of mild, moderate, moderate-to-severe or severe asthma. In particular, the methods, antibodies for uses, uses, composition, medicaments and kits of the present invention are useful in treatment of moderate-to-severe or severe asthma. The term “mild asthma” refers to well controlled with GINA Step 1 or Step 2 treatment, i.e. with as-needed controller medication alone, or with low-intensity maintenance controller treatment such as low dose ICS, leukotriene receptor agonists or chromones (Global strategy for asthma management and prevention, GINA report 2019, page 35). The term “moderate asthma” refers to well controlled with GINA Step 3 treatment, e.g. low dose ICS-LABA (Global strategy for asthma management and prevention, GINA report 2019, page 35). The term “severe asthma” refers to asthma that requires GINA Step 4 or 5 treatment, e.g. high dose ICS-LABA, to prevent it from becoming “uncontrolled” or asthma that remains “uncontrolled” despite the treatment (Global strategy for asthma management and prevention, GINA report 2019, page 35). The term “moderate-to-severe asthma” refers to both, moderate and severe asthma.

Suitably, the methods, antibodies for uses, uses, compositions, medicaments and kits of the present invention are useful in treatment of uncontrolled asthma. In preferred embodiments, the methods, antibodies for uses, uses, compositions, medicaments and kits of the present invention are useful in treatment of severe uncontrolled asthma. The term “uncontrolled asthma” refers to asthma that is uncontrolled on GINA Step 4 or 5 treatment, e.g., with poor symptom control.

Suitably, asthma severity can be assessed retrospectively from the level of treatment required to control symptoms and exacerbations. It can be assessed once the patient has been on controller treatment for several months and, if appropriate, treatment step down has been attempted to find the patient's minimum effective level of treatment. Asthma severity is not a static feature and may change over months or years.

In a further embodiment, the methods, antibodies for uses, uses, compositions, medicaments and kits of the present invention are useful in treatment of bronchitis asthma or asthmatic bronchitis, exercise-induced asthma, occupational asthma, asthma induced following bacterial or viral infection.

The term “bronchitis asthma” or “asthmatic bronchitis” is bronchitis that happens as a result of asthma.

The term “exercise-induced asthma” refers to asthma induced due to physical exercises, in particular in athletes competing at a high level. Asthma in athletes is commonly characterized by less correlation between symptoms and pulmonary function; higher lung volumes and expiratory flows; less eosinophilic airway inflammation; more difficulty in controlling symptoms; and some improvement in airway dysfunction after cessation of training. Exercise-induced asthma can occur due to exposure to air pollutants, allergens and chlorine levels in pools during training periods.

The term “occupational asthma” refers to asthma acquired due to exposures at work. Asthma may be induced or (more commonly) aggravated by exposure to allergens or other sensitizing agents at work, or sometimes from a single, massive exposure.

The term “asthma induced following bacterial infection” refers to asthma, which onset and exacerbation is associated with microbial infections, e.g., Chronic Mycoplasma pneumonia and Chlamydia pneumonia infections.

The term “asthma induced following viral infection” refers to asthma, which onset and exacerbation is associated with viral infections, e.g., respiratory virus, e.g., rhinovirus, parainfluenza virus.

Efficacy in the treatment of asthma is evidenced by reduced frequency or severity of symptomatic attack, e.g., of acute asthmatic or bronchoconstrictor attack, improvement in lung function or improved airway hyperreactivity. It may further be evidenced by reduced requirement for other, symptomatic therapy, i.e., therapy for or intended to restrict or abort symptomatic attack when it occurs, e.g., anti-inflammatory (e.g., corticosteroid) or bronchodilatory. Treatment benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 a.m., i.e., at a time normally substantially distant from any previously administered symptomatic asthma therapy.

Other inflammatory or obstructive airway diseases and conditions to which the present invention is applicable include acute lung injury (ALI), adult respiratory distress syndrome (ARDS), chronic obstructive pulmonary, airway or lung disease (COPD, COAD or COLD), including chronic bronchitis or dyspnea associated therewith, emphysema, as well as exacerbation of airway hyperreactivity consequent to other drug therapy, in particular, other inhaled drug therapy.

In one aspect, the present invention provides a method of treating COPD, e.g., emphysema and/or chronic bronchitis or reducing the frequency and/or severity of COPD exacerbations or improving COPD symptoms, e.g., reducing EXACT—Respiratory Symptoms (E-RS) score and/or reducing COPD Assessment Test (CAT) score and/or a reducing of St. George's Respiratory Questionnaire (SGRQ) Score in a subject and/or reducing mMRC (Modified Medical Research Council) Dyspnea score.

As used herein, the term “COPD” refers to a chronic inflammatory lung disease that causes obstructed airflow from the lungs. The term “COPD” as used herein refers to asthma of all phenotypes and endotypes in all severity levels regardless the disease control status, such as emphysema and/or chronic bronchitis. Symptoms of COPD include breathing difficulty, cough, mucus (sputum) production and wheezing. COPD is characterized by poorly reversible, progressive airways obstruction. The two most common conditions of COPD are chronic bronchitis and emphysema. Chronic bronchitis is a long-term inflammation of the bronchi, which results in increased production of mucus, as well as other changes. These changes may result in breathing problems, frequent infections, cough, and disability. Chronic bronchitis is associated with hyperplasia and hypertrophy of the mucus secreting glands of the submucosa in the large cartilaginous airways. Goblet cell hyperplasia, mucosal and submucosal inflammatory cell infiltration, edema, fibrosis, mucus plugs and increased smooth muscle are all found in the terminal and respiratory bronchioles. The small airways are known to be a major site of airway obstruction. Emphysema is characterized by destruction of the alveolar wall and loss of lung elasticity. Emphysema is a chronic lung condition in which alveoli may be destroyed, narrowed, collapsed, stretched and/or over-inflated. This can cause a decrease in respiratory function and breathlessness. Damage to the air sacs is irreversible and results in permanent “holes” in the lung tissue. The major pathophysiology of COPD is irreversible obstruction of the airway with progressive lung function decline, especially in patients with continuous exposure to risk factors such as cigarette smoke, biomass smoke exposure, air pollution, exposure to particles, asthma and airway hyper-reactivity, chronic bronchitis and infections.

The term “COPD exacerbation” refers to an acute or sub-acute worsening in COPD symptoms and lung function from the patient's usual status, in particular characterized by acute worsening of dyspnea, e.g., an acute or sub-acute worsening of dyspnoea (>5 using a 0-scale), and/or cough and sputum production, and/or increased sputum purulence.

Suitably, the methods, antibodies for uses, uses, compositions, medicaments and kits of the present invention are useful in treatment of COPD in a patient with low eosinophil count, e.g., wherein a patient has a screening blood eosinophil count of <300 cells/μl, in particular at the time of start of treatment or at the time of diagnosis. Suitably, the methods, antibodies for uses, uses, compositions, medicaments and kits of the present invention are useful in treatment of COPD in a patient with high eosinophil count, e.g., wherein a patient has a screening blood eosinophil count of >300 cells/μl, in particular at the time of start of treatment or at the time of diagnosis. Suitably, the methods, antibodies for uses, uses, compositions, medicaments and kits of the present invention are useful in treatment of COPD in a patient with low Th2 profile, e.g., wherein a patient has IgE<100 IU/mL and Blood Eosinophil Count <140 cells/μl, in particular at the time of start of treatment or at the time of diagnosis. Suitably, the methods, antibodies for uses, uses, compositions, medicaments and kits of the present invention are useful in treatment of COPD in a patient with high Th2 profile, e.g., wherein a patient has IgE>100 IU/mL and Blood Eosinophil Count >140 cells/μl, in particular at the time of start of treatment or at the time of diagnosis. The invention is also applicable to the treatment of bronchitis of whatever type or genesis including, e.g., acute, arachidic, catarrhal, croupus, chronic or phthinoid bronchitis.

Further inflammatory or obstructive airway diseases to which the present invention is applicable include pneumoconiosis (an inflammatory, commonly occupational, disease of the lungs, frequently accompanied by airway obstruction, whether chronic or acute, and occasioned by repeated inhalation of dusts) of whatever type or genesis including, e.g., aluminosis, anthracosis, asbestosis, chalicosis, ptilosis, siderosis, silicosis, tabacosis and byssinosis.

Having regard to their anti-inflammatory activity, in particular, in relation to inhibition of eosinophil activation, an anti-TSLP antibody or anti-TSLP antibody fragment, when provided at a specific concentration and/or at specific time intervals and/or by a specific route of administration as disclosed herein, is also useful in the treatment of eosinophil related disorders, e.g., eosinophilia, in particular, eosinophils-related disorders of the airway, e.g., involving morbid eosinophilic infiltration of pulmonary tissues including hypereosinophilia as it effects the airways and/or lungs, as well as, e.g., eosinophil-related disorders of the airways consequential or concomitant to Loffler's syndrome; eosinophilic pneumonia; parasitic, in particular, metazoan, infestation including tropical eosinophilia; bronchopulmonary aspergillosis; polyarteritis nodosa including Churg-Strauss syndrome; eosinophilic granuloma; and eosinophil-related disorders affecting the airways occasioned by drug-reaction.

The effectiveness of an anti-TSLP antibody or anti-TSLP antibody fragment, when provided at a specific concentration and/or at specific time intervals and/or by a specific route of administration as disclosed herein, in inhibiting inflammatory conditions, e.g., in inflammatory airway diseases, may be demonstrated in an animal model, e.g., a mouse or rat model, of airway inflammation or other inflammatory conditions, e.g., as described by Szarka et al., J Immunol Methods, Vol. 202, pp. 49-57 (1997); Renzi et al., Am Rev Respir Dis, Vol. 148, pp. 932-939 (1993); Tsuyuki et al., J Clin Invest, Vol. 96, pp. 2924-2931 (1995); Cernadas et al., Am J Respir Cell Mol Biol, Vol. 20, pp. 1-8 (1999); and Williams and Galli, J Exp Med, Vol. 192, pp. 455-462 (2000).

### Subject

As used herein, the term “subject” or “patient” includes any human or non-human animal. In a preferred embodiment, the subject is human. The term “non-human animal” includes all vertebrates, e.g., mammals and non-mammals, such as non-human primates, sheep, dogs, cats, horses, cows, chickens, amphibians, reptiles, etc. As used herein, a subject is “in need of” a treatment if such subject would benefit biologically, medically or in quality of life from such treatment.

In some embodiments of the present invention, the subject is an adult or adolescent. The term “adults” as used herein refers to a subject >18 years of age. The term “adolescent” as used herein refers to a subject >12 years and <18 years of age. In some embodiments of the present invention, the subject is a child. The term “child” as used herein refers to a subject <12 years of age.

In some embodiments, the present invention relates to treatment of an inflammatory or obstructive airway disease comprising administering to a subject in need thereof a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment, wherein the subject is on a background therapy of:
(a) medium or high dose corticosteroids, e.g., inhaled corticosteroids (ICS); or
(b) medium or high dose corticosteroids, e.g., inhaled corticosteroids (ICS), in combination with LABA (ICS/LABA); or
(c) medium or high dose corticosteroids, e.g., inhaled corticosteroids (ICS), in combination with LABA and up to two additional to ICS-LABA controllers, e.g., LTRA, theophylline or its derivatives or LAMA, e.g., ICS/LABA/LAMA.

In some more specific embodiments, the present invention relates to treatment of an inflammatory or obstructive airway disease comprising administering to a subject in need thereof a dose of about 0.5 mg to about 16 mg, e.g., about 1 mg to about 16 mg, about 2 mg to about 16 mg, about 2 mg to about 8 mg, in particular a dose of about 4 mg to about 8 mg, of an anti-TSLP antibody or anti-TSLP antibody fragment, wherein the subject is on background therapy of a combination of fluticasone furoate and vilanterol, e.g., o.d. with a fixed dose combination of 92 to 100 ug of fluticasone furoate and 22 to 25 ug of vilanterol, or o.d. with a fixed dose combination of 92 to 100 ug of fluticasone furoate and 22 to 25 ug of vilanterol with up to two additional controllers, e.g., LTRA, theophylline or its derivatives or LAMA (e.g., umeclidinium, glycopyrronium).

In some embodiments, the subject suitable for the purposes of the present invention is selected according to at least one of the following criteria:
(a) prior to treatment with the antibody or antibody fragment, the subject has a post-bronchodilator reversibility of FEV1>12% and >200 mL, e.g., a post-bronchodilator reversibility of FEV1>12% and >200 mL within 30 minutes after administration of 400 μg salbutamol/albuterol (or equivalent dose);
(b) prior to treatment with the antibody or antibody fragment, the subject has a spirometry with pre-bronchodilator FEV1>40% and <85%, e.g., pre-bronchodilator FEV1 value of >40% and <85% of the predicted normal after withholding bronchodilators;
(c) prior to treatment with the antibody or antibody fragment, the subject has an ACQ-5 score >1.5.

### Efficacy

Successful treatment of an inflammatory or obstructive airway disease, in particular asthma or COPD, may be demonstrated by one or more of the following parameters, each of which is able to be achieved by the methods, antibodies for uses, uses, compositions, medicaments and kits of the present invention. The effectiveness of the the methods, antibodies for uses, uses, compositions, medicaments and kits of the present invention may be assessed using various known methods and tools that measure an inflammatory or obstructive airway disease or its clinical response, e.g., asthma and/or asthma's clinical response, COPD and/or COPD's clinical response. Some examples include Spirometry (Forced Expiratory Volume in 1 second) (FEV1), peak expiratory flow rate (PEF), Daytime and nighttime asthma symptom score (captured in eDairy), ACQ-5 score, number of puffs of short-acting beta2-agonist (SABA) taken per day (captured in eDairy), rate of and severity of exacerbations, EXACT—Respiratory Symptoms (E-RS) score, COPD Assessment Test (CAT) score, a of St. George's Respiratory Questionnaire (SGRQ) score, mMRC (Modified Medical Research Council) Dyspnea score. The effectiveness of the the methods, antibodies for uses, uses, compositions, medicaments and kits of the present invention may be assessed using various known methods and tools that measure asthma and/or asthma's clinical response, such as Spirometry (Forced Expiratory Volume in 1 second) (FEV1), peak expiratory flow rate (PEF), Daytime and nighttime asthma symptom score (captured in eDairy), ACQ-5 score, number of puffs of short-acting beta2-agonist (SABA) taken per day (captured in eDairy). The effectiveness of the methods, antibodies for uses, uses, compositions, medicaments and kits of the present invention may be assessed using various known methods and tools that measure COPD and/or COPD's clinical response, such as Spirometry (Forced Expiratory Volume in 1 second) (FEV1), peak expiratory flow rate (PEF), rate of and severity of exacerbations, EXACT—Respiratory Symptoms (E-RS) score, COPD Assessment Test (CAT) score, a of St. George's Respiratory Questionnaire (SGRQ) score, mMRC (Modified Medical Research Council) Dyspnea score.

Clinical efficacy measurements related to the methods of treatment disclosed herein are outlined below.

In some embodiments, the methods, antibodies for uses, uses, compositions, medicaments and kits of the present invention lead to the subject achieving after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment at least one of the following:
a) an improvement in FVC;
b) an improvement in the FEV1;
c) an improvement of FeNO
d) an improvement in morning and evening peak expiratory flow rate (PEF);
e) an improvement of one or more symptoms of asthma, e.g., a reduction of the daytime and/or nighttime asthma symptom score (ADSD and/or ANSD), as measured by an asthma symptom diary;
f) a reduction in the ACQ-5 score;
g) a reduction of Asthma Quality of Life Questionnaire (AQLQ) score, e.g., AQLQ+12 score.

In some embodiments, treatment of an inflammatory or obstructive airway disease, in particular asthma, lead to the subject achieving after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment at least one of the following:
a) an improvement in FVC;
b) an improvement in the FEV1;
c) an improvement of FeNO
d) an improvement in morning and evening peak expiratory flow rate (PEF);
e) an improvement of one or more symptoms of asthma, e.g., a reduction of the daytime and/or nighttime asthma symptom score (ADSD and/or ANSD), as measured by an asthma symptom diary;
f) a reduction in the ACQ-5 score;
g) a reduction of Asthma Quality of Life Questionnaire (AQLQ) score, e.g., AQLQ+12 score;
h) a reduction in rate of and severity of exacerbations.

In some embodiments, treatment of an inflammatory or obstructive airway disease, in particular COPD, lead to the subject achieving after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment at least one of the following:
a) an improvement in FVC;
b) an improvement in the FEV1;
c) a reduction of the EXACT—Respiratory Symptoms (E-RS) score or E-RS score, in particular a reduction of the EXACT—Respiratory Symptoms (E-RS) score or E-RS score by at least 1.5 points from baseline;
d) a reduction of the COPD Assessment Test (CAT) score, in particular a reduction of CAT score by at least 2 points from baseline;
e) a reduction of St. George's Respiratory Questionnaire (SGRQ) score, in particular a reduction of SGQR score by at least 4 points from baseline;
f) a reduction of mMRC (Modified Medical Research Council) Dyspnea score, in particular a reduction of mMRC score by at least 1 point from baseline;
g) a reduction in rate of and severity of exacerbations.

Spirometry testing is performed according to the American Thoracic Society guidelines [SERIES “ATS/ERS TASK FORCE: STANDARDISATION OF LUNG FUNCTION TESTING” Edited by V. Brusasco, R. Crapo and G. Viegi Number 2 in this Series Standardisation of spirometry, Miller M R et el. Eur Respir J 2005; 26: 319-338]. Spirometry Reversibility Test is performed according to the American Thoracic Society guidelines.

Successful treatment of an inflammatory or obstructive airway disease, in particular asthma or COPD, may be demonstrated by the ability to achieve an increase in pulmonary function compared to before treatment, e.g., increased pulmonary function relative to the pulmonary function measured for said subject before treatment, in particular as measured by a pulmonary function test selected from the list of spirometry (e.g., in particular as measured by forced vital capacity (FVC), e.g., absolute or FVC % (forced vital capacity, expressed as a percentage of the normal expected value for a healthy individual of the same age and demographics) and forced expiratory volume in one second (FEV1), FEF25-75, FEV1/FVC, FEV3/FVC, 1-(FEV3/FVC), FEV6.

Herein, the forced vital capacity (FVC) is the maximal amount of air that the subject can forcibly exhale after taking a maximal inhalation. Predicted FVC is expressed as a percentage of the normal expected value, stratified by gender, age, height, and race (% FVC). An increase can be measured based on the predicted FVC based on a large patient population, on the FVC measured in a control population, or on the individual subject's FVC, e.g., predicted FVC, prior to administration of the anti-TSLP antibody of the disclosure or fragment thereof (baseline). In some embodiments, the methods, antibodies for uses, uses, compositions, medicaments and kits of the present invention described herein can increase the FVC as compared to the subject's baseline FVC, e.g., to the predicted FVC. In some embodiments, the methods, antibodies for uses, uses, compositions, medicaments and kits of the present invention provided herein can increase FVC, e.g., predicted FVC, compared to before treatment, e.g., increase in FVC relative to the FVC value for said subject before treatment, by at least 3% or at least 4% or at least 5% or at least 6% or at least 7% or at least 8% or at least 9%, at least 10%, at least 15%, at least 20%, at least 25%, at least 30%. In some embodiments of the invention, FVC, e.g., predicted FVC, is increased compared to before treatment, e.g., increase in FVC relative to the FVC value for said subject before treatment, after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

Herein, forced expiratory volume in one second (FEV1) is the volume exhaled during the first second of a forced expiratory maneuver started from the level of full inspiration. In different embodiments, the methods, antibodies for uses, uses, compositions, medicaments and kits provided herein can increase forced expiratory volume in one second (FEV1) in the subject. An increase can be measured based on the expected FEV1 based on a large patient population, on the FEV1 measured in a control population, or on the individual patient's FEV1 prior to administration of the anti-TSLP antibody of the disclosure or fragment thereof (baseline). In one embodiment, the methods, antibodies for uses, uses, compositions, medicaments and kits of the present invention can increase the FEV1, as compared to the patient's baseline FEV1. In some embodiments, the methods, antibodies for uses, uses, compositions, medicaments and kits provided herein can increase Forced Expiratory Volume in 1 Second (FEV1) compared to before treatment, e.g., increase in FEV1 relative to the FEV1 value for said subject before treatment, by at least 3% or at least 4% or at least 5% or at least 6% or at least 7% or at least 8% or at least 9%, at least 10%, at least 15%, at least 20%, at least 25%, at least 30%. In some embodiments of the invention, FEV1 is increased compared to before treatment, e.g., increase in FEV1 relative to the FEV1 value for said subject before treatment, after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

Clinical Outcome Assessments (COAs): The validated questionnaire (Asthma Control Questionnaire or ACQ 1999, Eur. Respir J; 902-7) is used and is administered at various timepoints during the study as depicted in the Assessment Schedule.

The ACQ-7 has 7 questions (woken at night by symptoms, wake in the mornings by symptoms, limitation of daily activities, shortness of breath, wheeze, FED1% predicted and daily rescue bronchodilator use). Patients are asked to recall how their asthma has been during the previous week and respond to the symptom and bronchodilator use questions on a 7-points scale (0=no impairment, 6=maximum impairment). Clinical staff score the FEV1% predicted on a 7-point scale. The questions are equally weighted and the ACQ score is the mean of the 7 questions and therefore between 0 (totally controlled) and 6 (severely uncontrolled. The ACQ has been fully validated for use in both clinical practice and clinical trials. As used herein, a patient with an inadequately controlled asthma has an ACQ score >1.0, preferably an ACQ score >1.5. A change from baseline of the ACQ score which is considered to be clinically relevant is a change or decrease in the ACQ score of at least 0.5, preferably at least 1, more preferably at least 1.5.

The ACQ-5 measures asthma symptom control and consists of 5 items (questions) on symptom assessment. All 5 questions of the ACQ-5 are equally weighted. Items are scored along a 7-point response scale, where 0=totally controlled and 6=severely uncontrolled. The total score is calculated as the mean of all five questions. A change from baseline of the ACQ score which is considered to be clinically relevant is a change or decrease in the ACQ score of at least 0.5, preferably at least 1, more preferably at least 1.5.

Daytime and nighttime asthma symptoms (ADSD and ANSD): In some embodiments, the methods, antibodies for uses, uses, compositions, medicaments and kits provided herein lead to a reduction in the ADSD and/or ANSD score.

eDiary for daily asthma symptoms, peak flow and rescue medication use: The study uses the asthma diary reported by Santanello et al 1997. This asthma diary was validated in studies of patients aged 18 to 65 years (Santanello et al 1997, Eur Respir J; 646-51). The diary was subsequently included as a measure in placebo-controlled studies of montelukast in patients aged 15 years and older (Reiss et al 1998, Arch Intern Med. 158(11): 1213-20, Malmstrom et al 1999, American Society of Internal medicine: 487) and shown to be responsive to both montelukast and inhaled beclomethasone therapy in this age range.

For scoring measurement, the daytime asthma symptom scale uses a range of response categories for each question from 0 to 6, indicating the least to the most asthma symptomatology. The nocturnal diary scale uses response categories ranging from 0 (indicating no awakening with asthma symptoms) to 3 (indicating awake all night).

Daytime symptom diary scale questions 1) How often did you experience asthma symptoms today? 0 (None of the time) to 6 (all of the time); 2) How much did your asthma symptoms bother you today? 0 (Not at all bothered) to 6 (severly bothered); 3) How much activity could you do today? 0 (More than usual activity) to 6 (Less than usual activity); 4) How often did your asthma affect your activities today? 0 (None of the time) to 6 (All of the time).

Nocturnal diary scale question 1) Did you wake up with asthma symptoms. (This can be awakening in the middle of the night or on awakening in the morning)? 0 (No), 1 (Once), 2 (More than once) and 3 (Awake “all night” Daily).

Daytime scale scores were computed as the average of the four questions on the daytime symptom scale. An overall diary score for the week is computed as the average of the daily daytime scale scores. Weekly average scores for the nocturnal diary scale are computed in a similar manner. A decrease in the weekly score for the daytime and nocturnal scales indicate an improvement in asthma symptoms. The change from baseline in the asthma scale scores is computed as the difference between the score prior to dosing with an anti-TSPT antibody of a fragment thereof and at week 12 of the active treatment phase with an anti-TSPT antibody of a fragment thereof.

All subjects are provided with a patient electronic diary (eDiary or eDiary/ePEF) to record daily asthma symptoms, PEF and SABA (salbutamol/albuterol) use. Subjects are instructed to routinely complete the patient diary twice daily—at the same time each morning and each evening, approximately 12 hours apart. The eDiary/ePEF recordings are to be reviewed at each clinic visit as detailed in the Assessment schedule.

The data captured in the patient eDiary is used in conjunction with the patient's asthma characteristics to monitor the patient's asthma. Subjects are instructed to call the study site if they experience symptoms of worsening asthma. Additionally, the eDiary is programmed to generate some alerts of signs of possible worsening asthma based on data collected. These alerts are sent to the subject and/or to investigator.

Daily symptom scores: The asthma diary contains daytime and nocturnal asthma symptom questions (Santanello et al 1997). The format of the electronically administered diary may vary.

Number of inhalations of rescue medication: The total number of inhalations of SABA (number of puffs taken in the previous 12 hours) is recorded in the morning and evening by the subject in the eDiary/ePEF.

Peak expiratory flow (PEF): In some embodiments, the methods, antibodies for uses, uses, compositions, medicaments and kits provided herein lead to an improvement in morning and evening peak expiratory flow rate (PEF).

PEF is a person's maximum speed of expiration, as measured with a peak flow meter. PEF is measured at consistent times for a subject in the morning and evening each day. It measures the airflow through the bronchi and thus the degree of obstruction in the airways. Peak expiratory flow is typically measured in units of liters per minute (L/min). To determine the significance of peak expiratory flow measurements, a comparison is made to reference (normal, predicted) values based on measurements taken from the general population. Various reference values have been published in the literature and vary by population, ethnic group, age, sex, height and weight of the patient. It is also expressed as a percentage of the usual or normal peak flow readings. A desired PEF for a patient is at least 80%, preferably at least 90% of the usual or normal peak flow rate for this particular patient. In some embodiments, the methods and uses provided herein can increase PEF compared to before treatment, e.g., increase in PEF relative to the PEF value for said subject before treatment, by at least 3% or at least 4% or at least 5% or at least 6% or at least 7% or at least 8% or at least 9%, at least 10%, at least 15%, at least 20%, at least 25%, at least 30%. In some embodiments of the invention, PEF is increased compared to before treatment, e.g., increase in PEF relative to the PEF value for said subject before treatment, after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment. In some embodiments of the invention, PEF is at least 80%, preferably at least 90% after at least 1 week, after at least 2 weeks, after at least 3 weeks, after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment. In some embodiments, there is an improvement in morning and evening peak expiratory flow rate (PEF).

In some embodiments, the methods, antibodies for uses, uses, compositions, medicaments and kits provided herein lead to a reduction of Asthma Quality of Life Questionnaire (AQLQ) score, e.g., AQLQ+12 score. The AQLQ+12 comprises a total of 32 individual questions that span a total of four domains: symptoms, activity limitation, emotional function, and environmental stimuli. Test-retest reliability, construct validity (cross-sectional and longitudinal), and responsiveness have been demonstrated. It takes about 4 to 5 minutes to complete. Patients are asked to recall their experiences during the previous 2 weeks and to score each item on a 7-point scale (7=not at all impaired to 1=severely impaired). The AQLQ+12 yields individual domain scores, which is the mean of all items in each domain, and an overall score, which is the mean of all 32 individual responses. Higher scores indicate less impairment in Health Related Quality of Life (HRQOL).

In some embodiments, the methods, antibodies for uses, uses, compositions, medicaments and kits provided herein lead to an improvement of FeNO (fractional exhaled nitric oxide).

The measures described above (spirometry, PEF-measurements, FeNO, diaries, records of rescue medication and patient reported outcomes ACQ) are well-established and widely used outcome measures in asthma trials.

E-RS is a patient-reported diary to assess the cardinal symptoms of COPD overall and through three symptom-specific domains: Cough and Sputum, Chest Symptoms, and Breathlessness. The Evaluating Respiratory Symptoms (E-RS™) scale includes 11 items from EXACT with a daily recall period.

The EXACT is a validated 14-item electronic questionnaire designed to detect the frequency, severity, and duration of exacerbations in patients with COPD (Leidy et al 2011, Leidy et al 2014a, Leidy et al 2014b). It is to be completed by the patient at the end of every day at bedtime in order to measure the underlying day-to-day variability of COPD, and detect worsening indicative of an exacerbation. Within the 14-item EXACT tool, the Evaluating Respiratory Symptoms (E-RS™) scale is based on the 11 respiratory symptom items (primary endpoint). These 11 items generate a total score, quantifying respiratory symptom severity overall, and 3 subscale scores assessing breathlessness, cough and sputum, and chest symptoms.

COPD Assessment Test (CAT) is a widely used in clinical practice and research and is reflected in GOLD as a tool for baseline stratification of COPD patients (Jones et al 2009). It captures disease/symptom burden with a daily recall period. It consists of 8 items, each presented as a semantic 6-point differential scale, providing a total score out of 40. A higher score indicates a worse health status. The result is immediately available without the need for any calculation, apart from summing the scores on individual items. Scores of 0-10, 11-20, 21-30 and 31-40 represent a mild, moderate, severe or very severe clinical impact of COPD upon the patient.

Modified Medical Research Council (mMRC) scale is widely used in clinical practice and research and is reflected in GOLD as a tool for baseline stratification of COPD patients as it assesses degree of baseline functional disability due to dyspnea. It has 5 levels, correlates with healthcare-associated quality of life, morbidity, and possibly mortality for patients with COPD. It is a self-rating tool to measure the degree of disability that breathlessness poses on day-to-day activities on a scale from 0 to 4: 0, no breathlessness except on strenuous exercise; 1, shortness of breath when hurrying on the level or walking up a slight hill; 2, walks slower than people of same age on the level because of breathlessness or has to stop to catch breath when walking at their own pace on the level; 3, stops for breath after walking ˜100 m or after few minutes on the level; and 4, too breathless to leave the house, or breathless when dressing or undressing.

St. George's Respiratory Questionnaire (SGRQ) is widely accepted regulatory endpoint to capture symptom disease burden in COPD (Jones el al 1992). It is a 50-item, self-administered measure for evaluating health status, with a subscale measuring the severity and impact of symptoms.

The measures described above (spirometry, diaries, records of rescue medication and patient reported outcomes, e.g., E-RS) are well-established and widely used outcome measures in COPD trials.

In some embodiments, the administration of an anti-TSLP antibody or a fragment thereof according to the methods of the present invention decreases eosinophils in blood, sputum, broncheoalveolar fluid, or lungs of the subject.

In some embodiments, the administration of an anti-TSLP antibody or a fragment thereof according to the methods of the present shifts cell counts in the subject from a Th2 high population to a Th2 low population.

In some embodiments, the administration of an anti-TSLP antibody or a fragment thereof reduces rate of and severity of exacerbations, e.g., delays the time to an asthma or COPD exacerbation compared to a subject not receiving the anti-TSLP antibody or antibody fragment thereof.

The term “asthma exacerbation” refers to a worsening of asthma that leads to any of the following: Use of systemic corticosteroids for at least 3 days; a single depo-injectable dose of corticosteroids is considered equivalent to a 3-day course of systemic corticosteroids; for subjects receiving maintenance OCS, a temporary doubling of the maintenance dose for at least 3 days qualifies; an ED visit due to asthma that required systemic corticosteroids (as per above); an inpatient hospitalization due to asthma. Additional measures associated with asthma exacerbations are also being examined to determine effect. These include hospitalizations related to asthma exacerbations (i.e., severe asthma exacerbations), time to first asthma exacerbation, and the proportion of subjects with one or more asthma exacerbation/severe asthma exacerbation.

The term “COPD exacerbation” refers to an acute or sub-acute worsening in COPD symptoms and lung function from the patient's usual status, in particular characterized by acute worsening of dyspnea, e.g., an acute or sub-acute worsening of dyspnoea (>5 using a 0-scale), and/or cough and sputum production, and/or increased sputum purulence.

### Anti-TSLP Antibody or Anti-TSLP Antibody Fragment

The various the methods, uses, compositions, medicaments and kits provided herein utilize an anti-TSLP antibody or anti-TSLP antibody fragment.

As used herein, “TSLP” (also known as “thymic stromal lymphopoietin”) refers to a cytokine produced by non-hematopoietic cells in response to proinflammatory stimuli. The human TSLP gene is mapped to chromosomal location 5q22.1, and the genomic sequence of TSLP gene can be found in GenBank at NC_000005.10. Due to alternative splicing, two TSLP isoforms are present in the human. The protein and mRNA sequences for the two human TSLP isoforms are listed in Table 1.

The longer TSLP isoform 1, is linked with the development of airway inflammatory disease (Headley et al., 2009, Journal of immunology 182, 1641-1647; Ying et al., 2005, Journal of immunology 174, 8183-8190). The term “TSLP” as used herein refers to TSLP isoform 1. As used herein, human TSLP protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the amino acid sequence of GenBank accession number NP_149024.1. A human TSLP nucleic acid sequence has over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleic acid sequence of GenBank accession number NM 033035.4.

TABLE 1
TSLP amino acid and mRNA sequences.

| | | GeneBank | |
|---|---|---|---|
| | | Accession | |
| Species | Isoform | No. | Sequence |
| Homo | TSLP | NP_ | MFPFALLYVLSVSFRKIFILQLV |
| sapiens | isoform | 149024.1 | GLVLTYDFTNCDFEKIKAAYLST |
| | 1 | | ISKDLITYMSGTKSTEFNNTVSC |
| | amino | | SNRPHCLTEIQSLTFNPTAGCAS |
| | acid | | LAKEMFAMKTKAALAIWCPGYSE |
| | | | TQINATQAMKKRRKRKVTTNKCL |
| | | | EQVSQLQGLWRRFNRPLLKQQ |
| | | | (SEQ ID NO: 21) |
| Homo | TSLP | NM_ | SEQ ID NO: 22 |
| sapiens | isoform | 033035.4 | |
| | 1 | | |
| | mRNA | | |
| Homo | TSLP | NP_ | MFAMKTKAALAIWCPGYSETQIN |
| sapiens | isoform | 612561.2 | ATQAMKKRRKRKVTTNKCLEQVS |
| | 2 | | QLQGLWRRFNRPLLKQQ |
| | amino | | (SEQ ID NO: 23) |
| | acid | | |
| Homo | TSLP | NM_ | SEQ ID NO: 24 |
| sapiens | isoform | 138551.4 | |
| | 2 | | |
| | mRNA | | |

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

The terms “antibody fragment”, “antigen-binding fragment”, “antigen-binding fragment thereof”, “antigen binding portion” of an antibody, and the like, as used herein, refer to one or more fragments of an intact antibody that retain the ability to specifically bind to a given antigen (e.g., TSLP). Antigen binding functions of an antibody can be performed by fragments of an intact antibody. Examples of binding fragments encompassed within the term “antigen binding portion” of an antibody include a Fab fragment, a monovalent fragment consisting of the VL, VH, CL and CH1 domains; a F (ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., 1989 Nature 341:544-546), which consists of a VH domain; and an isolated complementarity determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies. Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies). Antigen binding portions can be incorporated into single chain molecules comprising a pair of tandem Fv segments (VH—CH1-VH-CH1) which, together with complementary light chain polypeptides, form a pair of antigen binding regions (Zapata et al., 1995 Protein Eng. 8 (10):1057-1062; and U.S. Pat. No. 5,641,870).

In one embodiment, the antibody fragment disclosed herein is selected from the group consisting of a Fab, Fab′, F(ab′)2, scFv, minibody, or diabody. In a more specific embodiment, the antibody fragment is a Fab. In a more specific embodiment, the antibody fragment is human or humanized Fab.

In one embodiment, the antibody or antibody fragment disclosed herein is selected from the group consisting of a human antibody, humanized antibody, a chimeric antibody, a monoclonal antibody, a recombinant antibody, a human recombinant antibody. In one embodiment, the antibody is a human immunoglobulin. In one embodiment, the antibody fragment is human or humanized Fab, in particular human Fab.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody”, as used herein, includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948. The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant antibody” as used herein, includes antibodies that are prepared, expressed, created or isolated by recombinant means, e.g., by using recombinant antibody coding genes.

The phrase “recombinant human antibody” as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “humanized” antibody (or antigen-binding fragment thereof), as used herein, is an antibody (or antigen-binding fragment thereof) that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239: 1534-1536, 1988; Padlan, Molec. Immun., 28:489-498, 1991; and Padlan, Molec. Immun., 31: 169-217, 1994. Other examples of human engineering technology include, but is not limited to Xoma technology disclosed in U.S. Pat. No. 5,766,886.

The term “chimeric antibody” (or antigen-binding fragment thereof) is an antibody molecule (or antigen-binding fragment thereof) in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a K_{D} of 10-8 M or less, 10-9 M or less, or 10-10 M, or 10-11 M or less, or 10-12 M or less, or 10-13 M or less for a target antigen. However, high affinity binding can vary for other antibody isotypes. For example, high affinity binding for an IgM isotype refers to an antibody having a K_{D} of 10-7 M or less, or 10-8 M or less.

The term “K_{D}” is intended to refer to the dissociation rate of a particular antibody-antigen interaction. The term “K_{D}”, as used herein, is intended to refer to the dissociation constant, which is obtained from the ratio of k_{off} to kₒₙ (i.e., k_{off}/kₒₙ) and is expressed as a molar concentration (M). K_{D} values for antibodies can be determined using methods well established in the art. K_{D} and affinity are inversely related; the lower the K_{D} value, the higher the affinity of the antibody. A method for determining the K_{D} of an antibody is by using surface plasmon resonance, or using a biosensor system such as a Biacore© system. Suitably, the antibodies and antibody fragments disclosed herein bind human TSLP with a dissociation constant (K_{D}) of less than 100 pM, e.g., a K_{D} of less than 90 pM, less than 80 pM, less than 70 pM, less than 60 pM, less than 50 pM, less than 40 pM, less than 30 pM, less than 20 pM, less than 10 pM. In some embodiments, the antibodies and antibody fragments disclosed herein bind human TSLP with a dissociation constant (K_{D}) of less than 10 pM.

The antibody or antibody fragment disclosed herein include a heavy chain CDR1 (HCDR1), a heavy chain CDR2 (HCDR2), a heavy chain CDR3 (HCDR3), and a light chain CDR1 (LCDR1), a light chain CDR2 (LCDR2), and a light chain CDR3 (LCDR3). Suitably, the antibody or antibody fragment disclosed herein include a heavy chain variable region comprising HCDR1, HCDR2, and HCDR3 and a light chain variable region comprising LCDR1, LCDR2, and LCDR3. Suitably, the antibody or antibody disclosed herein may include a full length heavy chain sequence and a full length light chain sequence. In some embodiments, the antibody or antibody fragment is a Fab.

The terms “complementarity determining regions” and “CDRs” as used herein refer to the amino acid residues of an antibody or antigen-binding fragment that are responsible for antigen binding. The CDR regions are delineated using the Kabat system (Kabat et al. 1991 Sequences of Proteins of Immunological Interest, Fifth Edition, U.S. Department of Health and Human Services, NIH Publication No. 91-3242), or using the Chothia system (Chothia et al. 1987 J. Mol. Biol. 196: 901-917; and Al-Lazikani et al. 1997 J. Mol. Biol. 273: 927-948), or by combining both Kabat and Chothia definitions, such that, the CDRs may comprise some or all of the amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

The term “Fe region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a Ckappa/lambda region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

In a specific embodiment, the antibody or antibody fragment useful in the methods, uses, compositions, medicaments and kits provided herein is selected from any one of the following:
a) an antibody or antibody fragment that comprises:
   a HCDR1 comprising the amino acid sequence of SEQ ID NO: 4;
   a HCDR2 comprising the amino acid sequence of SEQ ID NO: 2;
   a HCDR3 comprising the amino acid sequence of SEQ ID NO: 3;
   a LCDR1 comprising the amino acid sequence of SEQ ID NO: 11;
   a LCDR2 comprising the amino acid sequence of SEQ ID NO: 12; and
   a LCDR3 comprising the amino acid sequence of SEQ ID NO: 13,
   in particular wherein the HCDR1, HCDR2, HCDR3, LCDR1, LCDR2 and
   LCDR3 are defined according to the Kabat numbering scheme;
b) an antibody or antibody fragment that comprises:
   a HCDR1 comprising the amino acid sequence of SEQ ID NO: 5;
   a HCDR2 comprising the amino acid sequence of SEQ ID NO: 6;
   a HCDR3 comprising the amino acid sequence of SEQ ID NO: 3;
   a LCDR1 comprising the amino acid sequence of SEQ ID NO: 14;
   a LCDR2 comprising the amino acid sequence of SEQ ID NO: 15; and
   a LCDR3 comprising the amino acid sequence of SEQ ID NO: 16,
   in particular wherein the HCDR1, HCDR2, HCDR3, LCDR1, LCDR2 and LCDR3 are defined according to the Chothia numbering scheme;
   and
c) an antibody or antibody fragment that comprises
   a HCDR1 comprising the amino acid sequence of SEQ ID NO: 1;
   a HCDR2 comprising the amino acid sequence of SEQ ID NO: 2;
   a HCDR3 comprising the amino acid sequence of SEQ ID NO: 3;
   a LCDR1 comprising the amino acid sequence of SEQ ID NO: 11;
   a LCDR2 comprising the amino acid sequence of SEQ ID NO: 12; and
   a LCDR3 comprising the amino acid sequence of SEQ ID NO: 13,
   in particular wherein the HCDR1, HCDR2, HCDR3, LCDR1, LCDR2 and LCDR3 are defined according to the combination of the Chothia and Kabat numbering schemes.

In a more specific embodiment, the antibody or antibody fragment useful in the methods, uses, compositions, medicaments and kits provided herein is selected from any one of the following:
a) an antibody or antibody fragment that comprises:
   a HCDR1 consisting of the amino acid sequence of SEQ ID NO: 4;
   a HCDR2 consisting of the amino acid sequence of SEQ ID NO: 2;
   a HCDR3 consisting of the amino acid sequence of SEQ ID NO: 3;
   a LCDR1 consisting of the amino acid sequence of SEQ ID NO: 11;
   a LCDR2 consisting of the amino acid sequence of SEQ ID NO: 12; and
   a LCDR3 consisting of the amino acid sequence of SEQ ID NO: 13,
   in particular wherein the HCDR1, HCDR2, HCDR3, LCDR1, LCDR2 and LCDR3 are defined according to the Kabat numbering scheme;
b) an antibody or antibody fragment that comprises:
   a HCDR1 consisting of the amino acid sequence of SEQ ID NO: 5;
   a HCDR2 consisting of the amino acid sequence of SEQ ID NO: 6;
   a HCDR3 consisting of the amino acid sequence of SEQ ID NO: 3;
   a LCDR1 consisting of the amino acid sequence of SEQ ID NO: 14;
   a LCDR2 consisting of the amino acid sequence of SEQ ID NO: 15; and
   a LCDR3 consisting of the amino acid sequence of SEQ ID NO: 16,
   in particular wherein the HCDR1, HCDR2, HCDR3, LCDR1, LCDR2 and LCDR3 are defined according to the Chothia numbering scheme;
   and
c) an antibody or antibody fragment that comprises
   a HCDR1 consisting of the amino acid sequence of SEQ ID NO: 1;
   a HCDR2 consisting of the amino acid sequence of SEQ ID NO: 2;
   a HCDR3 consisting of the amino acid sequence of SEQ ID NO: 3;
   a LCDR1 consisting of the amino acid sequence of SEQ ID NO: 11;
   a LCDR2 consisting of the amino acid sequence of SEQ ID NO: 12; and
   a LCDR3 consisting of the amino acid sequence of SEQ ID NO: 13,
   in particular wherein the HCDR1, HCDR2, HCDR3, LCDR1, LCDR2 and LCDR3 are defined according to the combination of the Chothia and Kabat numbering schemes.

In a specific embodiment, the antibody or antibody fragment useful in the methods, uses, compositions, medicaments and kits provided herein is an antibody fragment selected from the group consisting of a Fab, Fab′, F(ab′)2, scFv, minibody, or diabody, in particular wherein the antibody fragment is a Fab, in particular human or humanized Fab.

In a more specific embodiment, the antibody or antibody fragment useful in the methods, uses, compositions, medicaments and kits provided herein comprises (or consists of)
a) a heavy chain variable region comprising (or consisting of)
   i. the amino acid sequence of SEQ ID NO: 7, or
   ii. a sequence at least 90% identical to SEQ ID NO: 7, or
   iii. a conservative variant of SEQ ID NO: 7; and
b) a light chain variable region comprising (or consisting of)
   i. the amino acid sequence of SEQ ID NO: 17, or
   ii. a sequence at least 90% identical to SEQ ID NO: 17, or
   iii. a conservative variant of SEQ ID NO: 17.

In a more specific embodiment, the antibody or antibody fragment useful in the methods, uses, compositions, medicaments and kits provided herein comprises (or consists of):
a) a heavy chain comprising (or consisting of)
   i. the amino acid sequence of SEQ ID NO: 9, or
   ii. a sequence at least 90% identical to SEQ ID NO: 9, or
   iii. a conservative variant of SEQ ID NO: 9, and
b) a light chain comprising (or consisting of)
   i. the amino acid sequence of SEQ ID NO: 19, or
   ii. a sequence at least 90% identical to SEQ ID NO: 19, or
   iii. a conservative variant of SEQ ID NO: 19.

In a specific embodiment, the antibody or antibody fragment useful in the methods, uses, compositions, medicaments and kits provided herein is administered at a dose of 2 mg to 16 mg, e.g., 2 mg or 4 mg or 8 mg or 16 mg, at an interval of once a week or once a day, in particular once a day.

The term “conservative sequence modifications” refers to amino acid modifications that do not significantly affect or alter the binding characteristics of the antibody or antibody fragment containing the amino acid sequence. Such conservative modifications include amino acid substitutions, additions, and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a molecule, such as an antibody or antibody fragment, of the invention can be replaced with other amino acid residues from the same side chain family and the altered molecule can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

Table 2 lists the sequences of suitable TSLP-binding antibodies and Fabs.

TABLE 2
Amino acid sequences of anti-TSLP Fab 1 (CSJ117).

| anti-TSLP Fab1 | | |
|---|---|---|
| (CSJ117) | | |
| SEQ ID NO: 1 | HCDR1 (Combined) | GFTFSDYWMH |
| SEQ ID NO: 2 | HCDR2 (Combined) | HIKSKTDAGTTDYAAPVKG |
| SEQ ID NO: 3 | HCDR3 (Combined) | EIYYYAFDS |
| SEQ ID NO: 4 | HCDR1 (Kabat) | DYWMH |
| SEQ ID NO: 2 | HCDR2 (Kabat) | HIKSKTDAGTTDYAAPVKG |
| SEQ ID NO: 3 | HCDR3 (Kabat) | EIYYYAFDS |
| SEQ ID NO: 5 | HCDR1 (Chothia) | GFTFSDY |
| SEQ ID NO: 6 | HCDR2 (Chothia) | KSKTDAGT |
| SEQ ID NO: 3 | HCDR3 (Chothia) | EIYYYAFDS |
| SEQ ID NO: 7 | VH | EVQLVESGGGLVKPGGSLRLSCAASGFTFSDYWM |
| | | HWVRQAPGKGLEWVGHIKSKTDAGTTDYAAPVK |
| | | GRFTISRDDSKNTLYLQMNSLKTEDTAVYYCAREI |
| | | YYYAFDSWGQGTLVTVSS |
| SEQ ID NO: 8 | VH DNA | GAGGTGCAGCTGGTGGAATCAGGCGGCGGACTG |
| | | GTCAAGCCTGGCGGTAGCCTGAGACTGAGCTGCG |
| | | CTGCTAGTGGCTTCACCTTTAGCGACTACTGGAT |
| | | GCACTGGGTCAGACAGGCCCCTGGTAAAGGCCTG |
| | | GAGTGGGTCGGACACATTAAGTCTAAGACCGAC |
| | | GCCGGCACTACCGACTACGCCGCTCCTGTGAAGG |
| | | GCCGGTTCACTATCTCTAGGGACGACTCTAAGAA |
| | | CACCCTGTACCTGCAGATGAATAGCCTGAAAACC |
| | | GAGGACACCGCCGTCTACTACTGCGCTAGAGAGA |
| | | TCTACTACTACGCCTTCGATAGCTGGGGTCAGGG |
| | | CACCCTGGTCACCGTGTCTAGC |
| SEQ ID NO: 9 | Heavy Chain | EVQLVESGGGLVKPGGSLRLSCAASGFTFSDYWM |
| | | HWVRQAPGKGLEWVGHIKSKTDAGTTDYAAPVK |
| | | GRFTISRDDSKNTLYLQMNSLKTEDTAVYYCAREI |
| | | YYYAFDSWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKRVEPKSC |
| SEQ ID NO: 10 | Heavy Chain DNA | GAGGTGCAGCTGGTGGAATCAGGCGGCGGACTG |
| | | GTCAAGCCTGGCGGTAGCCTGAGACTGAGCTGCG |
| | | CTGCTAGTGGCTTCACCTTTAGCGACTACTGGAT |
| | | GCACTGGGTCAGACAGGCCCCTGGTAAAGGCCTG |
| | | GAGTGGGTCGGACACATTAAGTCTAAGACCGAC |
| | | GCCGGCACTACCGACTACGCCGCTCCTGTGAAGG |
| | | GCCGGTTCACTATCTCTAGGGACGACTCTAAGAA |
| | | CACCCTGTACCTGCAGATGAATAGCCTGAAAACC |
| | | GAGGACACCGCCGTCTACTACTGCGCTAGAGAGA |
| | | TCTACTACTACGCCTTCGATAGCTGGGGTCAGGG |
| | | CACCCTGGTCACCGTGTCTAGCGCTAGCACTAAG |
| | | GGCCCCTCCGTGTTCCCTCTGGCCCCTTCCAGCA |
| | | AGTCTACCTCTGGCGGCACCGCTGCTCTGGGCTG |
| | | CCTGGTGAAGGACTACTTCCCTGAGCCTGTGACA |
| | | GTGTCCTGGAACTCTGGCGCCCTGACCTCCGGCG |
| | | TGCACACCTTCCCTGCCGTGCTGCAGTCCTCCGG |
| | | CCTGTACTCCCTGTCCTCCGTGGTGACAGTGCCTT |
| | | CCTCCAGCCTGGGCACCCAGACCTATATCTGCAA |
| | | CGTGAACCACAAGCCTTCCAACACCAAGGTGGA |
| | | CAAGCGGGTGGAGCCTAAGTCATGC |
| SEQ ID NO: 11 | LCDR1 (Combined) | SGDNIGSKYVH |
| SEQ ID NO: 12 | LCDR2 (Combined) | GDNERPS |
| SEQ ID NO: 13 | LCDR3 (Combined) | QAADWVDFYV |
| SEQ ID NO: 11 | LCDR1 (Kabat) | SGDNIGSKYVH |
| SEQ ID NO: 12 | LCDR2 (Kabat) | GDNERPS |
| SEQ ID NO: 13 | LCDR3 (Kabat) | QAADWVDFYV |
| SEQ ID NO: 14 | LCDR1 (Chothia) | DNIGSKY |
| SEQ ID NO: 15 | LCDR2 (Chothia) | GDN |
| SEQ ID NO: 16 | LCDR3 (Chothia) | ADWVDFY |
| SEQ ID NO: 17 | VL | SYELTQPLSVSVALGQTARITCSGDNIGSKYVHWY |
| | | QQKPGQAPVLVIYGDNERPSGIPERFSGSNSGNTAT |
| | | LTISRAQAGDEADYYCQAADWVDFYVFGGGTKLT |
| | | VL |
| SEQ ID NO: 18 | VL DNA | AGCTACGAGCTGACTCAGCCCCTGAGCGTCAGCG |
| | | TGGCCCTGGGTCAGACCGCTAGAATCACCTGTAG |
| | | CGGCGATAATATCGGCTCTAAATACGTGCACTGG |
| | | TATCAGCAGAAGCCCGGTCAGGCCCCCGTGCTGG |
| | | TGATCTACGGCGATAACGAGCGGCCTAGCGGAAT |
| | | CCCCGAGCGGTTTAGCGGCTCTAATAGCGGTAAC |
| | | ACCGCTACCCTGACTATCTCTAGGGCTCAGGCCG |
| | | GCGACGAGGCCGACTACTACTGTCAGGCCGCCG |
| | | ACTGGGTGGACTTCTACGTGTTCGGCGGAGGCAC |
| | | TAAGCTGACCGTGCTG |
| SEQ ID NO: 19 | Light Chain | SYELTQPLSVSVALGQTARITCSGDNIGSKYVHWY |
| | | QQKPGQAPVLVIYGDNERPSGIPERFSGSNSGNTAT |
| | | LTISRAQAGDEADYYCQAADWVDFYVFGGGTKLT |
| | | VLGQPKAAPSVTLFPPSSEELQANKATLVCLISDFY |
| | | PGAVTVAWKADSSPVKAGVETTTPSKQSNNKYAA |
| | | SSYLSLTPEQWKSHRSYSCQVTHEGSTVEKTVAPTE |
| | | CS |
| SEQ ID NO: 20 | Light Chain DNA | AGCTACGAGCTGACTCAGCCCCTGAGCGTCAGCG |
| | | TGGCCCTGGGTCAGACCGCTAGAATCACCTGTAG |
| | | CGGCGATAATATCGGCTCTAAATACGTGCACTGG |
| | | TATCAGCAGAAGCCCGGTCAGGCCCCCGTGCTGG |
| | | TGATCTACGGCGATAACGAGCGGCCTAGCGGAAT |
| | | CCCCGAGCGGTTTAGCGGCTCTAATAGCGGTAAC |
| | | ACCGCTACCCTGACTATCTCTAGGGCTCAGGCCG |
| | | GCGACGAGGCCGACTACTACTGTCAGGCCGCCG |
| | | ACTGGGTGGACTTCTACGTGTTCGGCGGAGGCAC |
| | | TAAGCTGACCGTGCTGGGTCAGCCTAAGGCTGCC |
| | | CCCAGCGTGACCCTGTTCCCCCCCAGCAGCGAGG |
| | | AGCTGCAGGCCAACAAGGCCACCCTGGTGTGCCT |
| | | GATCAGCGACTTCTACCCAGGCGCCGTGACCGTG |
| | | GCCTGGAAGGCCGACAGCAGCCCCGTGAAGGCC |
| | | GGCGTGGAGACCACCACCCCCAGCAAGCAGAGC |
| | | AACAACAAGTACGCCGCCAGCAGCTACCTGAGC |
| | | CTGACCCCCGAGCAGTGGAAGAGCCACAGGTCC |
| | | TACAGCTGCCAGGTGACCCACGAGGGCAGCACC |
| | | GTGGAAAAGACCGTGGCCCCAACCGAGTGCAGC |

Suitably, the antibodies and antibody fragments suitable in the methods, uses, compositions, medicaments and kits provided herein comprise (or alternatively, consist of) a VH amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have been mutated (wherein a mutation is, as various non-limiting examples, an addition, substitution or deletion).

Suitably, the antibodies and antibody fragments suitable in the methods, uses, compositions, medicaments and kits provided herein comprise (or alternatively, consist of) a VL amino acid sequence listed in Table 2, wherein no more than about 1, 2, 3, 4, 5, 6, 7, 8, 9, 10, 11, 12, 13, 14, 15, 16, 17, 18, 19, or 20 amino acids in a framework sequence (for example, a sequence which is not a CDR) have been mutated (wherein a mutation is, as various non-limiting examples, an addition, substitution or deletion).

In addition, the antibodies and antibody fragments suitable in the methods, uses, compositions, medicaments and kits provided herein may include amino acids that have been mutated, yet have at least 60, 70, 80, 90, 91, 92, 93, 94, 95, 96, 97, 98 or 99 percent identity in the CDR regions with the CDR regions depicted in the sequences described in Table 2 and are able to bind to TSLP. Suitably, the antibodies and antibody fragments suitable in the methods, uses, compositions, medicaments and kits provided herein may include mutant amino acid sequences wherein no more than 1, 2, 3, 4 or 5 amino acids have been mutated in the CDR regions when compared with the CDR regions depicted in the sequences described in Table 2.

Other anti-TSLP antibody or anti-TSLP antibody fragment suitable in the methods, uses, compositions, medicaments and kits provided herein include, but are not limited to antibodies described in WO09/035577 and WO2018/191479 (in particular, tezepelumab (AMG157; MEDI9929) or a fragment thereof), WO 2016/142426, WO 2010/017468, US20170066823, US20120020988 and U.S. Pat. No. 8,637,019, incorporated herein by reference, some of which are described below in Table 3.

In a specific embodiment, the antibody or antibody fragment useful in the methods, uses, compositions, medicaments and kits provided herein is an antibody or antibody fragment that comprises: a HCDR1 comprising the amino acid sequence of SEQ ID NO: 28; a HCDR2 comprising the amino acid sequence of SEQ ID NO: 29; a HCDR3 comprising the amino acid sequence of SEQ ID NO: 30; a LCDR1 comprising the amino acid sequence of SEQ ID NO: 25; a LCDR2 comprising the amino acid sequence of SEQ ID NO: 26; and a LCDR3 comprising the amino acid sequence of SEQ ID NO: 27. Suitably, the antibody or antibody fragment useful in the methods, uses, compositions, medicaments and kits provided herein is an antibody or antibody fragment that comprises: a HCDR1 consisting of the amino acid sequence of SEQ ID NO: 28; a HCDR2 consisting of the amino acid sequence of SEQ ID NO: 29; a HCDR3 consisting of the amino acid sequence of SEQ ID NO: 30; a LCDR1 consisting of the amino acid sequence of SEQ ID NO: 25; a LCDR2 consisting of the amino acid sequence of SEQ ID NO: 26; and a LCDR3 consisting of the amino acid sequence of SEQ ID NO: 27. In a more specific embodiment, the antibody or antibody fragment useful in the methods, uses, compositions, medicaments and kits provided herein comprises (or consists of):
a) a heavy chain variable region comprising (or consisting of)
   i. the amino acid sequence of SEQ ID NO: 32, or
   ii. a sequence at least 90% identical to SEQ ID NO: 32, or
   iii. a conservative variant of SEQ ID NO: 32; and
b) a light chain variable region comprising (or consisting of)
   i. the amino acid sequence of SEQ ID NO: 31, or
   ii. a sequence at least 90% identical to SEQ ID NO: 31, or
   iii. a conservative variant of SEQ ID NO: 31.

In a more specific embodiment, the antibody or antibody fragment useful in the methods, uses, compositions, medicaments and kits provided herein comprises (or consists of):
a) a heavy chain comprising (or consisting of)
   i. the amino acid sequence of SEQ ID NO: 33, or
   ii. a sequence at least 90% identical to SEQ ID NO: 33, or
   iii. a conservative variant of SEQ ID NO: 33, and
b) a light chain comprising (or consisting of)
   i. the amino acid sequence of SEQ ID NO: 34, or
   ii. a sequence at least 90% identical to SEQ ID NO: 34, or
   iii. a conservative variant of SEQ ID NO: 34.

In a specific embodiment, the antibody or antibody fragment useful in the methods, uses, compositions, medicaments and kits provided herein is tezepelumab. In one embodiment, the antibody or antibody fragment useful in the methods, uses, compositions, medicaments and kits provided herein is tezepelumab and is administered at a dose of 70 mg to 280 mg, e.g., 70 mg or 210 mg or 280 mg, at an interval of every 2 weeks, in particular every 4 weeks.

TABLE 3
Anti-TSLP antibodies.

| | | |
|---|---|---|
| AMG157 | | |
| SEQ ID NO: 25 | LCDR1 | GGNNLGSKSVH |
| SEQ ID NO: 26 | LCDR2 | DDSDRPS |
| SEQ ID NO: 27 | LCDR3 | QVWDSSSDHVV |
| SEQ ID NO: 28 | HCDR1 | TYGMH |
| SEQ ID NO: 29 | HCDR2 | VIWYDGSNKHYADSVK |
| SEQ ID NO: 30 | HCDR3 | APQWELVHEAFDI |
| SEQ ID NO: 31 | VL | SYVLTQPPSVSVAPGQTARITCGGNNLGSKSVHW |
| | | YQQKPGQAPVLVVYDDSDRPSWIPERFSGSNSGN |
| | | TATLTISRGEAGDEADYYCQVWDSSSDHVVFGGG |
| | | TKLTVL |
| SEQ ID NO: 32 | VH | QMQLVESGGGVVQPGRSLRLSCAASGFTFRTYGM |
| | | HWVRQAPGKGLEWVAVIWYDGSNKHYADSVKG |
| | | RFTITRDNSKNTLNLQMNSLRAEDTAVYYCARAP |
| | | QWELVHEAFDIWGQGTMVTVSS |
| SEQ ID NO: 33 | Full Heavy | QMQLVESGGGVVQPGRSLRLSCAASGFTFRTYGM |
| | Chain | HWVRQAPGKGLEWVAVIWYDGSNKHYADSVKG |
| | | RFTITRDNSKNTLNLQMNSLRAEDTAVYYCARAP |
| | | QWELVHEAFDIWGQGTMVTVSSASTKGPSVFPLA |
| | | PCSRSTSESTAALGCLVKDYFPEPVTVSWNSGALT |
| | | SGVHTFPAVLQSSGLYSLSSVVTVPSSNFGTQTYT |
| | | CNVDHKPSNTKVDKTVERKCCVECPPCPAPPVAG |
| | | PSVFLFPPKPKDTLMISRTPEVTCVVVDVSHEDPE |
| | | VQFNWYVDGVEVHNAKTKPREEQFNSTFRVVSV |
| | | LTVVHQDWLNGKEYKCKVSNKGLPAPIEKTISKT |
| | | KGQPREPQVYTLPPSREEMTKNQVSLTCLVKGFY |
| | | PSDIAVEWESNGQPENNYKTTPPMLDSDGSFFLYS |
| | | KLTVDKSRWQQGNVFSCSVMHEALHNHYTQKSL |
| | | SLSPGK |
| SEQ ID NO: 34 | Full Light | SYVLTQPPSVSVAPGQTARITCGGNNLGSKSVHW |
| | Chain | YQQKPGQAPVLVVYDDSDRPSWIPERFSGSNSGN |
| | | TATLTISRGEAGDEADYYCQVWDSSSDHVVFGGG |
| | | TKLTVLGQPKAAPSVTLFPPSSEELQANKATLVCL |
| | | ISDFYPGAVTVAWKADSSPVKAGVETTTPSKQSN |
| | | NKYAASSYLSLTPEQWKSHRSYSCQVTHEGSTVE |
| | | KTVAPTECS |
| WO 2016/142426 | | |
| SEQ ID NO: 35 | Single | DIQMTQSPSSLSASVGDRVTITCRASRPIRNWLDW |
| | variable | YQQKPGKAPKLLIWGASHLQSGVPSRFSGSGSGT |
| | domain | DFTLTISSLQPEDFATYYCVQIGEDPVTFGQGTKV |
| | | EIK |
| SEQ ID NO: 36 | CDR 1 | RASRPIRNWLD |
| SEQ ID NO: 37 | CDR 2 | GASHLQS |
| SEQ ID NO: 38 | CDR 2 | LLIWGASHLQ |
| SEQ ID NO: 39 | CDR 3 | VQIGEDPVT |
| US2012/0020988 | | |
| SEQ ID NO: 40 | HCDR 1 | GFSITTSGYYWT |
| SEQ ID NO: 41 | HCDR 1 | GFSITTSGYYWS |
| SEQ ID NO: 42 | HCDR 2 | YIGYNSKTYYNPSLKS |
| SEQ ID NO: 43 | HCDR 2 | YIGYNSKTYYNPALKS |
| SEQ ID NO: 44 | HCDR 3 | SLYGGYKDAFDS |
| SEQ ID NO: 45 | LCDR 1 | KASQSIGTSLH |
| SEQ ID NO: 46 | LCDR 1 | RASQSIGTSLH |
| SEQ ID NO: 47 | LCDR 2 | FASRSIT |
| SEQ ID NO: 48 | LCDR 2 | FASRLQS |
| SEQ ID NO: 49 | LCDR 2 | FASRSIS |
| SEQ ID NO: 50 | LCDR 3 | QQSPGFPPT |
| SEQ ID NO: 51 | VH-1 | QIQLQESGPGLVKPSQSLSLTCSVTGFSITTSGYYW |
| | | TWIRQFPGKKLEWMGYIGYNSKTYYNPSLKSRISI |
| | | TRDTSKNQFLLHLNSVTTEDTATYYCARSLYGGY |
| | | KDAFDSWGQGTLVTVSS |
| SEQ ID NO: 52 | VL-2 | DVVLTQTPATLSAIPGERVTMTCKASQSIGTSLHW |
| | | YQHRPNETPRLLIKFASRSITGIPSRFSGSGSGTDFT |
| | | LGINNLEAEDFAIYYCQQSPGFPPTFGSGTKLEINR |
| SEQ ID NO: 53 | VH-2 | QVQLVESGGGLVKPGGSLRLSCAASGFSITTSGYY |
| | | WTWIRQFPGKGLEWMGYIGYNSKTYYNPALKSRI |
| | | TISRDNAKNSLYLQMNSLRAEDTAVYYCARSLYG |
| | | GYKDAFDSWGQGTLVTVSS |
| SEQ ID NO: 54 | VL-2 | DIQMTQSPSSLSASVGDRVTITCRASQSIGTSLHW |
| | | YQQKPGKAPKLLIKFASRLQSGVPSRFSGSGSGTD |
| | | FTLTISSLQPEDFATYYCQQSPGFPPTFGQGTKLEI |
| | | KR |
| SEQ ID NO: 55 | VH-3 | QVQLVESGGGLVKPGGSLRLSCAASGFSITTSGYY |
| | | WTWIRQFPGKGLEWMGYIGYNSKTYYNPALKSRI |
| | | TISRDNAKNSLYLQMNSLRAEDTAVYYCARSLYG |
| | | GYKDAFDSWGQGTLVTVSS |
| SEQ ID NO: 56 | VL-3 | EIVLTQSPDFQSVTPKEKVTITCRASQSIGTSLHWY |
| | | QQKPDQSPKLLIKFASRSISGVPSRFSGSGSGTDFT |
| | | LTINSLEAEDAATYYCQQSPGFPPTFGQGTKLEIKR |
| SEQ ID NO: 57 | VH-4 | QVQLQESGPGLVKPSQTLSLTCTVSGFSITTSGYY |
| | | WSWIRQHPGKGLEWIGYIGYNSKTYYSPSLKSRV |
| | | TISRDTSKNQFSLKLSSVTAADTAVYYCARSLYGG |
| | | YKDAFDSWGQGTLVTVSS |
| SEQ ID NO: 58 | VL-4 | DIQMTQSPSSLSASVGDRVTITCRASQSIGTSLHW |
| | | YQHRPGETPKLLIKFASRSISGVPSRFSGSGSGTDF |
| | | TLTISSLQPEDFATYYCQQSPGFPPTFGQGTKLEIK |
| | | R |
| SEQ ID NO: 59 | VH-5 | QVQLVESGGGLVKPGGSLRLSCAASGFSITTSGYY |
| | | WTWIRQFPGKGLEWMGYIGYNSKTYYNPALKSRI |
| | | TISRDNAKNSLYLQMNSLRAEDTAVYYCARSLYG |
| | | GYKDAFDSWGQGTLVTVSS |
| SEQ ID NO: 60 | VL-5 | EIVMTQSPATLSVSPGERATLSCRASQSIGTSLHW |
| | | YQQKPGQAPRLLIKFASRSITGIPARFSGSGSGTEF |
| | | TLTISSLQSEDFAVYYCQQSPGFPPTFGQGTKLEIK |
| | | R |
| US8637019 | | |
| SEQ ID NO: 61 | HCDR1 | GYIFTDYAMH |
| SEQ ID NO: 62 | HCDR2 | TFIPLLDTSDYAQKFQ |
| SEQ ID NO: 63 | HCDR3 | MGVTHSYVMDA |
| SEQ ID NO: 64 | LCDR1 | RASQPISISVH |
| SEQ ID NO: 65 | LCDR2 | FASQSIS |
| SEQ ID NO: 66 | LCDR3 | QQTFSLPYT |
| SEQ ID NO: 67 | VH | QVQLVQSGAEVKKPGASVKVSCKASGYIFTDYA |
| | | MHWVRQAPGQGLEWMGTFIPLLDTSDYAQKFQG |
| | | RVTMTADTSTSTAYMELRSLRSDDTAVYYCARM |
| | | GVTHSYVMDAWGQGTLVTVSS |
| SEQ ID NO: 68 | VL | EIVLTQSPGTLSLSPGERATLSCRASQPISISVHWY |
| | | QQKPGQAPRLLIYFASQSISGIPDRFSGSGSGTDFT |
| | | LTISRLEPEDFAVYYCQQTFSLPYTFGQGTKVEIKR |
| | | T |
| SEQ ID NO: 69 | HC | QVQLVQSGAEVKKPGASVKVSCKASGYIFTDYA |
| | | MHWVRQAPGQGLEWMGTFIPLLDTSDYAQKFQG |
| | | RVTMTADTSTSTAYMELRSLRSDDTAVYYCARM |
| | | GVTHSYVMDAWGQGTLVTVSSASTKGPSVFPLAP |
| | | SSKSTSGGTAALGCLVKDYFPEPVTVSWNSGALT |
| | | SGVHTFPAVLQSSGLYSLSSVVTVPSSSLGTQTYIC |
| | | NVNHKPSNTKVDKKVEPKSCDKTHTCPPCPAPEL |
| | | LGGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTI |
| | | SKAKGQPREPQVYTLPPSRDELTKNQVSLTCLVK |
| | | GFYPSDIAVEWESNGQPENNYKTTPPVLDSDGSFF |
| | | LYSKLTVDKSRWQQGNVFSCSVMHEALHNHYTQ |
| | | KSLSLSPGK |
| SEQ ID NO: 70 | LC | EIVLTQSPGTLSLSPGERATLSCRASQPISISVHWY |
| | | QQKPGQAPRLLIYFASQSISGIPDRFSGSGSGTDFT |
| | | LTISRLEPEDFAVYYCQQTFSLPYTFGQGTKVEIKR |
| | | TVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNR |
| | | GEC |

### Co-Administration

Suitably, the anti-TSLP antibody of the disclosure or fragment thereof may be administered to the subject in combination (concomitantly) with a second agent, including but not limited to an anti-inflammatory agent or asthma therapy or COPD therapy.

The terms “co-administration” or “combined administration” or the like as utilized herein are meant to encompass the administration of one or more agents described herein together with a selected combination partner to a single subject in need thereof (e.g., a patient or subject), and are intended to include treatment regimens in which the agents are not necessarily administered by the same route of administration and/or at the same time.

When co-administered with one or more additional asthma or COPD agent(s), the anti-TSLP antibody of the disclosure or fragment thereof may be administered either simultaneously with the other agent, or sequentially. If administered sequentially, the attending physician will decide on the appropriate sequence of administering the compound of the anti-TSLP antibody of the disclosure or fragment thereof in combination with other agents and the appropriate dosages for co-delivery.

Exemplary second agents include, but are not limited to:
a) corticosteroids, e.g., inhaled corticosteroids (ICS) (e.g., fluticasone furoate, budesonide, beclometasone) or oral corticosteroids;
b) bronchodilators, e.g., long-acting J2 agonist (LABA) (e.g., vilanterol, formoterol), short-acting J2 agonist (SABA) (e.g., salbuterol, levosalbuterol), anticholinergics, e.g., ipratropium, tiotropium, aclidinium and glycopyrronium;
c) leukotriene receptor antagonists (LTRA), e.g., theophylline or its derivatives, Montekulast, Zafirlukast and Pranlukast;
d) long-acting anti-muscarinics (LAMA), e.g., Tiotropium, umeclidinium, glycopyrronium;
e) cromones;
f) antihistamines;
g) antileukotrienes; and
h) PDE-4 inhibitors.

In some embodiments, the methods of the present invention comprise administering (e.g., co-administering) to a subject the anti-TSLP antibody of the disclosure or fragment thereof and a second agent, such as anti-inflammatory, bronchodilatory or antihistamine drug substances, in particular in the treatment of obstructive or inflammatory airway diseases, such as those mentioned hereinbefore, e.g., as potentiators of therapeutic activity of such drugs or as a means of reducing required dosaging or potential side effects of such drugs. Suitably, the anti-TSLP antibody of the disclosure or fragment thereof may be mixed with the other agent in a fixed pharmaceutical composition or it may be administered separately, before, simultaneously with or after the other drug substance. Accordingly the invention includes a combination of the anti-TSLP antibody of the disclosure or fragment thereof with an anti-inflammatory, bronchodilatory, antihistamine or anti-tussive agent, said anti-TSLP antibody of the disclosure or fragment thereof and said agent being in the same or different pharmaceutical composition.

Suitably, the second agent is selected from the group comprising or consisting of an anti-inflammatory drug, such as corticosteroids, e.g., inhaled corticosteroids (ICS) (e.g., fluticasone furoate, beclometasone) or oral corticosteroids; bronchodilators, e.g., long-acting β2 agonist (LABA) (e.g., vilanterol, formoterol), short-acting β2 agonist (SABA), anticholinergics, e.g., ipratropium, tiotropium, aclidinium and glycopyrronium; leukotriene receptor antagonists (LTRA), e.g., theophylline or its derivatives; long-acting anti-muscarinics (LAMA, e.g., umeclidinium, glycopyrronium); cromones; antihistamines; antileukotrienes; and PDE-4 inhibitors. Suitably, the anti-TSLP antibody of the disclosure or a fragment thereof may be used in the treatment of an inflammatory or obstructive airway disease, e.g., in the treatment of COPD or asthma in combination with ICS or ICS/LABA or ICS/LABA/LAMA (e.g., a combination of fluticasone furoate, vilanterol, and umeclidinium (Trelegy®), or a beclometasone, formoterol, and glycopyrronium (Trimbow®)).

Suitably, the second agent is an anti-inflammatory drug. Suitable anti-inflammatory drugs include steroids, in particular, glucocorticosteroids or corticosteroids, such as budesonide, beclometasone, beclamethasone dipropionate, fluticasone furoate, fluticasone propionate, ciclesonide or mometasone furoate; or steroids, described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445 and WO 03/072592. Suitably, the second agent is an inhaled corticosteroid (ICS) or oral corticosetroid. Suitably, the second agent is non-steroidal glucocorticoid receptor agonists, such as those described in WO 00/00531, WO 02/10143, WO 03/082280, WO 03/082787, WO 03/104195 and WO 04/005229; LTB4 antagonists, such as those described in U.S. Pat. No. 5,451,700; LTD4 antagonists, such as montelukast and zafirlukast; PDE4 inhibitors, such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659 (Parke-Davis), AWD-12-281 (Asta Medica), CDC-801 (Celgene), SelCID™ CC-10004 (Celgene), KW-4490 (Kyowa Hakko Kogyo), WO 03/104204, WO 03/104205, WO 04/000814, WO 04/000839 and WO 04/005258 (Merck), as well as those described in WO 98/18796 and WO 03/39544; A2a agonists, such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462 and WO 03/086408; A2b antagonists, such as those described in WO 02/42298; and beta (β)-2-adrenoceptor agonists, such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, as well as compounds (in free or salt or solvate form) of formula (I) of WO 04/16601. Further β-2-adrenoreceptor agonists include compounds, such as those described in WO 99/64035, WO 01/42193, WO 01/83462, WO 02/066422, WO 02/070490, WO 02/076933, WO 2004/011416 and US 2002/0055651. Suitably, the second agent is a bronchodilatory drug, such as an anticholinergic or an antimuscarinic agent, in particular, ipratropium bromide, oxitropium bromide, tiotropium salts and CHF 4226 (Chiesi), but also those described in WO 01/04118, WO 02/51841, WO 02/53564, WO 03/00840, WO 03/87094, WO 04/05285, WO 02/00652, WO 03/33495, WO 03/53966, EP 0424021, U.S. Pat. Nos. 5,171,744 and 3,714,357. Suitably, the second agent is an antihistamine drug, such as cetirizine hydrochloride, acetaminophen, clemastine fumarate, promethazine, loratidine, desloratidine, diphenhydramine and fexofenadine hydrochloride.

Combinations of the anti-TSLP antibody of the disclosure or a fragment thereof and steroids, β-2 agonists, PDE4 inhibitors or LTD4 antagonists may be used in the treatment of an inflammatory or obstructive airway disease, e.g., in the treatment of COPD or asthma. Combinations of anti-TSLP antibody of the disclosure or a fragment thereof and anticholinergic or antimuscarinic agents, PDE4 inhibitors, dopamine receptor agonists or LTB4 antagonists may be used in the treatment of an inflammatory or obstructive airway disease, e.g., in the treatment of asthma or COPD.

Other useful combinations of anti-TSLP antibody of the disclosure or a fragment thereof with anti-inflammatory drugs are those with antagonists of chemokine receptors, e.g., CCR-1, CCR-2, CCR-3, CCR-4, CCR-5, CCR-6, CCR-7, CCR-8, CCR-9, CCR-10, CXCR1, CXCR2, CXCR3, CXCR4 and CXCR5; in particular useful are CCR-3 antagonists, such as those described in WO 2002/026723, especially 4-{3-[(S)-4-(3,4-dichlorobenzyl)-morpholin-2-ylmethyl]-ureidomethyl}-benzamide and those described in WO 2003/077907, WO 2003/007939 and WO 2002/102775. Also especially useful are CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770); and CCR-5 antagonists, described in U.S. Pat. No. 6,166,037, WO 00/66558 and WO 00/66559.

In some embodiments, the administration of anti-TSLP antibody or fragment thereof according to the methods of present invention reduces frequency or levels of co-administered therapy in the subject. Optionally, co-administered therapy comprises an agent selected from the group consisting of corticosteroids, e.g., inhaled corticosteroids (ICS) (e.g., fluticasone furoate, beclometasone) or oral corticosteroids; bronchodilators, e.g., long-acting 32 agonist (LABA) (e.g., vilanterol, formoterol), short-acting 32 agonist (SABA), anticholinergics, e.g., ipratropium, tiotropium, aclidinium and glycopyrronium; leukotriene receptor antagonists (LTRA), e.g., theophylline or its derivatives; long-acting anti-muscarinics (LAMA, e.g., umeclidinium, glycopyrronium); cromones; antihistamines; antileukotrienes; and PDE-4 inhibitors. Suitably, the anti-TSLP antibody of the disclosure or a fragment thereof may be used in the treatment of an inflammatory or obstructive airway disease, e.g., in the treatment of COPD or asthma in combination with ICS or ICS/LABA or ICS/LABA/LAMA (e.g., a combination of fluticasone furoate, vilanterol, and umeclidinium (Trelegy®), or a beclometasone, formoterol, and glycopyrronium (Trimbow®)).

In some embodiments, the administration of anti-TSLP antibody or fragment thereof according to the methods of present invention eliminates the need for corticosteroid therapy.

### Pharmaceutical Composition and Medicament

The various methods, compositions, medicaments and kits disclosed herein utilize an anti-TSLP antibody or anti-TSLP antibody fragment, wherein the anti-TSLP antibody or antibody fragment is disposed in a pharmaceutical composition. In one embodiment the pharmaceutical composition disclosed herein further comprises a pharmaceutically acceptable carrier.

The phrase “pharmaceutically acceptable” refers to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. The phrase “pharmaceutically acceptable composition” or “pharmaceutical composition” or “pharmaceutically acceptable formulations” or “pharmaceutical formulations” refers to a composition consisting of those compounds, materials, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

It will be appreciated that the pharmaceutical compositions described herein may be suitable for use in methods, compositions, formulations, medicaments and kits disclosed herein.

Pharmaceutical compositions typically include a pharmaceutically acceptable excipient. A pharmaceutically acceptable excipient can includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. For example, for administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

The pharmaceutical compositions provided herein are formulated for targeted delivery to the respiratory tract of a subject, especially the lung of the subject. Such formulation can bypass deposition of the active ingredient in the upper respiratory tract of the subject, thereby minimizing tolerability or safety issues associated with drug deposition in the mouth and throat. In some embodiments, the pharmaceutical compositions provided herein are formulated as a dry powder formulation. Such dry powder formulation can include the active ingredient, a shell-forming excipient, a glass-forming excipient, and a buffer.

In one aspect, the present invention provides a pharmaceutical composition comprising an anti-TSLP antibody or anti-TSLP antibody fragment and a pharmaceutically acceptable carrier.

In one aspect, the present invention provides a pharmaceutical composition comprising spray-dried particles comprising:
i. a core comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the composition, in particular about 3% to about 50% by weight based on the total weight (w/w) of the composition;
   and
ii. a shell comprising trileucine, wherein trileucine is about 1% to about 25% (w/w), in particular about 10% to about 15% (w/w) of the composition.

The amount of the anti-TSLP antibody or anti-TSLP antibody fragment in the pharmaceutical formulation can be adjusted to deliver a therapeutically effective amount of the anti-TSLP antibody or anti-TSLP antibody fragment per unit dose to achieve the desired result. In practice, this will vary widely depending upon the particular ingredient, its activity, the severity of the condition to be treated, the patient population, dosing requirements, the desired therapeutic effect and the relative amounts of additives contained in the composition. The composition will generally contain anywhere from about 1% to about 70% (w/w) of the anti-TSLP antibody or anti-TSLP antibody fragment, in particular about 3% by weight to about 50% by weight of the anti-TSLP antibody or anti-TSLP antibody fragment, e.g., about 1% (w/w), about 2% (w/w), about 3-3.5% (w/w), about 3.5-4.5% (w/w), about 4.5-5.5% (w/w), about 5.5-6.5% (w/w), about 6.5-7.5% (w/w), about 7.5-8.5% (w/w), about 8.5-9.5% (w/w), about 8.5%-10% (w/w), about 10% to 15% (w/w) (e.g., about 9.5-10.5% (w/w), about 10.5-11.5% (w/w), about 11.5-12.5% (w/w), about 12-13% (w/w), about 13-14% (w/w), about 14-15% (w/w)), about 15%-20% (w/w), about 20% to about 50% (w/w/) (e.g., 20% (w/w), about 25% (w/w), about 30% (w/w), about 35% (w/w), about 40% (w/w), about 45% (w/w), about 50% (w/w)). More specifically, the composition of the invention contains from 1% to 70% (w/w) of the anti-TSLP antibody or anti-TSLP antibody fragment, in particular 3% by weight to 50% by weight of anti-TSLP antibody or anti-TSLP antibody fragment, e.g., 1% (w/w), 2% (w/w), 3-3.5% (w/w), 3.5-4.5% (w/w), 4.5-5.5% (w/w), 5.5-6.5% (w/w), 6.5-7.5% (w/w), 7.5-8.5% (w/w), 8.5-9.5% (w/w), 8.5%-10% (w/w), 10% to 15% (w/w) (e.g., 9.5-10.5% (w/w), 10.5-11.5% (w/w), 11.5-12.5% (w/w), 12-13% (w/w), 13-14% (w/w), 14-15% (w/w)), 15%-20% (w/w), 20% to 50% (w/w/) (e.g., 20% (w/w), 25% (w/w), 30% (w/w), 35% (w/w), 40% (w/w), 45% (w/w), 50% (w/w)). Suitably, the composition of the invention contains from about 1% by weight to about 70% by weight of anti-TSLP antibody or anti-TSLP antibody fragment, in particular about 3% by weight to about 50% by weight of anti-TSLP antibody or anti-TSLP antibody fragment, e.g., about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% to about 50% (w/w/) (e.g., about 20% (w/w), about 25% (w/w), about 30% (w/w), about 35% (w/w), about 40% (w/w), about 45% (w/w), about 50% (w/w). More specifically, the composition of the invention contains from 1% by weight to 70% by weight of anti-TSLP antibody or anti-TSLP antibody fragment, in particular from 3% by weight to 50% by weight of anti-TSLP antibody or anti-TSLP antibody fragment, e.g., 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% to 50% (w/w/) (e.g., 20% (w/w), 25% (w/w), 30% (w/w), 35% (w/w), 40% (w/w), 45% (w/w), 50% (w/w).

In some embodiments, the composition contains about 3-3.5% (w/w), about 5% to 10% (w/w) (e.g., about 5% (w/w), about 6-6.5% (w/w), about 10%), about 10% to about 20% (w/w) (e.g., about 10%, about 12.5% (w/w), about 15% (w/w), about 20%), about 20% to about 50% (w/w) (e.g., about 20%, about 25% (w/w), about 40%, about 50% (w/w)) anti-TSLP antibody or anti-TSLP antibody fragment. In more specific embodiments, the composition contains 3-3.5% (w/w), 5% to 10% (w/w) (e.g., 5% (w/w), 6-6.5% (w/w), 10%), 10% to 20% (w/w) (e.g., 10%, 12.5% (w/w), 15% (w/w), 20%), 20% to 50% (w/w) (e.g., 20%, 25% (w/w), 40%, 50% (w/w)) anti-TSLP antibody or anti-TSLP antibody fragment.

Suitably, the pharmaceutical compositions of the present invention are suitable for delivery of a therapeutically effective amount of the anti-TSLP antibody or anti-TSLP antibody fragment per unit dose to achieve the desired result, in particular are suitable for delivery of about 0.5 mg to about 16 mg, e.g., about 1 mg to about 16 mg, about 2 mg to about 16 mg, about 2 mg to about 8 mg, in particular about 4 mg to about 8 mg, of the anti-TSLP antibody or anti-TSLP antibody fragment. In one embodiment, the composition of the invention comprises about 0.5 mg to about 16 mg, e.g., about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, of the anti-TSLP antibody or anti-TSLP antibody fragment. Thus, in one embodiment, the composition of the invention comprises 0.5 mg to 16 mg, e.g., 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, of the anti-TSLP antibody or anti-TSLP antibody fragment.

It is to be understood that more than one active ingredient, e.g., anti-TSLP antibody or fragment thereof, may be incorporated into the compositions described herein.

In one embodiment, the anti-TSLP antibody or anti-TSLP antibody fragment of the pharmaceutical composition of the present invention is selected from the group consisting of a human antibody, humanized antibody, a chimeric antibody, a monoclonal antibody, a recombinant antibody.

In one embodiment, the anti-TSLP antibody or anti-TSLP antibody fragment of the pharmaceutical composition of the present invention is selected from the group consisting of a Fab, Fab′, F(ab′)2, scFv, minibody, or diabody, in particular wherein the antibody fragment is a Fab, in particular human or humanized Fab.

In one embodiment, the anti-TSLP antibody or anti-TSLP antibody fragment of the pharmaceutical composition of the present invention is a human immunoglobulin.

In a specific embodiment, the anti-TSLP antibody or anti-TSLP antibody fragment of the pharmaceutical composition of the present invention is selected from any one of the following:
a. an antibody or antibody fragment that comprises:
   a HCDR1 comprising the amino acid sequence of SEQ ID NO: 4;
   a HCDR2 comprising the amino acid sequence of SEQ ID NO: 2;
   a HCDR3 comprising the amino acid sequence of SEQ ID NO: 3;
   a LCDR1 comprising the amino acid sequence of SEQ ID NO: 11;
   a LCDR2 comprising the amino acid sequence of SEQ ID NO: 12; and
   a LCDR3) comprising the amino acid sequence of SEQ ID NO: 13,
   in particular wherein the HCDR1, HCDR2, HCDR3, LCDR1, LCDR2 and LCDR3 are defined according to the Kabat numbering scheme;
b. an antibody or antibody fragment that comprises:
   a HCDR1 comprising the amino acid sequence of SEQ ID NO: 5;
   a HCDR2 comprising the amino acid sequence of SEQ ID NO: 6;
   a HCDR3 comprising the amino acid sequence of SEQ ID NO: 3;
   a LCDR1 comprising the amino acid sequence of SEQ ID NO: 14;
   a LCDR2 comprising the amino acid sequence of SEQ ID NO: 15; and
   a LCDR3 comprising the amino acid sequence of SEQ ID NO: 16,
   in particular wherein the HCDR1, HCDR2, HCDR3, LCDR1, LCDR2 and LCDR3 are defined according to the Chothia numbering scheme;
   and
c. an antibody or antibody fragment that comprises
   a HCDR1 comprising the amino acid sequence of SEQ ID NO: 1;
   a HCDR2 comprising the amino acid sequence of SEQ ID NO: 2;
   a HCDR3 comprising the amino acid sequence of SEQ ID NO: 3;
   a LCDR1 comprising the amino acid sequence of SEQ ID NO: 11;
   a LCDR2 comprising the amino acid sequence of SEQ ID NO: 12; and
   a LCDR3 comprising the amino acid sequence of SEQ ID NO: 13,
   in particular wherein the HCDR1, HCDR2, HCDR3, LCDR1, LCDR2 and LCDR3 are defined according to the combination of the Chothia and Kabat numbering schemes.

In a more specific embodiment, the antibody or antibody fragment of the pharmaceutical composition of the present invention comprises
a) a heavy chain variable region comprising
   i. the amino acid sequence of SEQ ID NO: 7, or
   ii. a sequence at least 90% identical to SEQ ID NO: 7, or
   iii. a conservative variant of SEQ ID NO: 7; and
b) a light chain variable region comprising
   i. the amino acid sequence of SEQ ID NO: 17, or
   ii. a sequence at least 90% identical to SEQ ID NO: 17, or
   iii. a conservative variant of SEQ ID NO: 17.

In a more specific embodiment, the antibody or antibody fragment of the pharmaceutical composition of the present invention comprises
a) a heavy chain comprising
   i. the amino acid sequence of SEQ ID NO: 9, or
   ii. a sequence at least 90% identical to SEQ ID NO: 9, or
   iii. a conservative variant of SEQ ID NO: 9, and
b) a light chain comprising
   i. the amino acid sequence of SEQ ID NO: 19, or
   ii. a sequence at least 90% identical to SEQ ID NO: 19, or
   iii. a conservative variant of SEQ ID NO: 19.

The compositions described herein contain a pharmaceutically acceptable hydrophobic shell-forming excipient. Shell-forming excipients are surface active agents that enhance dispersibility of spray-dried powders. The hydrophobic shell-forming excipient may take various forms that will depend at least to some extent on the composition and intended use of the dry powder formulation. Suitable pharmaceutically acceptable hydrophobic excipients may, in general, be selected from the group consisting of long-chain phospholipids, hydrophobic amino acids and peptides, and long chain fatty acid soaps. The compositions described herein include a shell-forming excipient trileucine.

By control of the formulation and process, it is possible for the surface of the spray-dried particles to be comprised primarily of the shell-forming excipient. Surface concentrations may be greater than 70%, such as greater than 75% or 80% or 85%. In some embodiments, the surface is comprised of greater than 90% shell-forming excipient, or greater than 95% or 98% or 99% hydrophobic excipient. For potent active ingredients, it is not uncommon for the surface to be comprised of more than 95% shell-forming excipient.

The shell-forming excipient facilitates development of a rugous particle morphology. This means the particle morphology is porous, wrinkled, corrugated or creased rather than smooth. This means the interior and/or the exterior surface of the inhalable medicament particles are at least in part rugous. This rugosity is useful for providing high delivery efficiency, dose consistency and drug targeting by improving powder fluidization and dispersibility. Increases in particle rugosity result in decreases in inter-particle cohesive forces as a result of an inability of the particles to approach to within van der Waals contact. The decreases in cohesive forces are sufficient to dramatically improve powder fluidization and dispersion in ensembles of rugous particles.

The pharmaceutical compositions of the present invention comprise a shell comprising trileucine, wherein trileucine is about 1% to about 25% (w/w), in particular about 10% to about 15% (w/w) of the composition. In one embodiment, the pharmaceutical compositions of the present invention comprise a shell comprising trileucine, wherein trileucine is about 10% (w/w) of the composition, preferably 10% (w/w) of the composition. In another embodiment, the pharmaceutical compositions of the present invention comprise a shell comprising trileucine, wherein trileucine is about 15% (w/w) of the composition, preferably 15% (w/w) of the composition. Trileucine improves the powder aerosolization behavior and dispersibility, providing higher delivered dose, which potentially allows for a reduction of the drug loading in the formulation while maintaining the amount delivered to the lungs.

Due to the short timescale of the drying event, active ingredients that are dissolved in the feedstock will be generally present as amorphous solids in the spray-dried drug product. The molecular mobility of an amorphous solid is significant when compared to that of its crystalline counterpart. Molecular mobility comprises long-range motions related to molecular diffusion as well as local motions such as bond rotations. The central principle in solid-state stabilization of amorphous materials is that molecular mobility leads to undesirable physical and chemical changes. Therefore, formulation strategies for amorphous materials usually focus on suppression of molecular mobility.

The compositions of the present invention contain a glass-forming excipient. Glass-forming excipients that suppress long-range molecular mobility include carbohydrates, amino acids, and buffers. In some embodiments, glass-forming excipients include: histidine, histidine HCl, sucrose, trehalose, mannitol, and sodium citrate. Thus some excipients, such as histidine, may be referred to as a buffer or a glass-forming excipient interchangeably. In preferred embodiments, the compositions of the present invention include trehalose. Trehalose is used as a stabilizer and is required to create an amorphous glass which helps to dramatically reduce molecular mobility and motions in the anti-TSLP antibody or antibody fragment, e.g., anti-TSLP Fab, thus preventing physicochemical changes (e.g., aggregation and chemical modification). In some embodiments, the compositions of the present invention comprise from about 10% to about 95% (w/w) trehalose, in particular about 20% to about 85% (w/w) trehalose, e.g., about 20% to about 25% (e.g., about 20%, about 24.5%, about 25%), about 30% to about 35% (e.g., about 30%, about 32.5%, about 35%), about 50% to about 60% (e.g., about 50%, about 55%, about 58%, about 60%), about 65% to about 85% (e.g., about 65%, about 70%, about 71%, about 72%, about 73%, about 74%, about 75%, about 76%, about 77%, about 78%, about 79%, about 80%, about 81%, about 82%, about 83%, about 84%, about 85%). In some embodiments, the compositions of the present invention comprise from 10% to 95% (w/w) trehalose, in particular 20% to 85% (w/w) trehalose, e.g., 20% to 25% (e.g., 20%, 24.5%, 25%), 30% to 35% (e.g., 30%, 32.5%, 35%), 50% to 60% (e.g., 50%, 55%, 58%, 60%), 65% to 85% (e.g., 65%, 70%, 71%, 72%, 73%, 74%, 75%, 76%, 77%, 78%, 79%, 80%, 81%, 82%, 83%, 84%, 85%).

In some embodiments, the pharmaceutical composition of the present invention has a ratio of trehalose: anti-TSLP antibody or anti-TSLP antibody fragment of greater than 0.4, greater than 0.5, greater than 0.6, greater than 0.65, greater than 1, greater than 1.5, greater than 2, greater than 3, greater than 4, greater than 5, greater than 10, greater than 15, greater than 20, greater than 25.

In a further embodiment, the present invention provides a pharmaceutical composition comprising spray-dried particles comprising:
i. a core comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the composition, in particular about 3% to about 50% by weight based on the total weight (w/w) of the composition;
   and
ii. a shell comprising trileucine, wherein trileucine is about 1% to about 25% (w/w), in particular about 10% to about 15% (w/w) of the composition; and wherein the composition, in particular the core of the composition, has a pH of 4.5% to 6.0%.

In a further embodiment, the present invention provides a pharmaceutical composition comprising spray-dried particles comprising:
i. a core comprising an anti-TSLP antibody or anti-TSLP antibody fragment, in particular an anti-TSLP antibody fragment, more particularly an anti-TSLP antibody Fab, wherein said anti-TSLP antibody or anti-TSLP antibody fragment is about 1% to about 70% (w/w) of the composition, in particular about 2% to about 50%, more particularly about 3% to about 50% by weight based on the total weight (w/w) of the composition;
   and
ii. a shell comprising trileucine, wherein trileucine is about 1% to about 25% (w/w), in particular about 10% to about 15% (w/w) of the composition; and further comprising a buffer;
   in particular wherein the composition, in particular the core of the composition, has a pH of 4.5% to 6.0%.

In one embodiment, the buffer comprises HCl. In one embodiment, the buffer comprises histidine. In one embodiment, the buffer comprises HCl and histidine. In some embodiments, the pharmaceutical composition of the invention comprises about 2% (w/w) to about 15% (w/w) histidine, in particular about 5% (w/w) to about 9% (w/w) histidine. In some embodiments, the pharmaceutical composition of the invention comprises about 0.5% (w/w) to about 4% (w/w) HCl, in particular about 1% (w/w) to about 2% (w/w) HCl.

In some embodiments, the pharmaceutical composition of the invention comprises about 2% (w/w) to about 50% (w/w) anti-TSLP antibody or anti-TSLP antibody fragment, in particular about 3% (w/w) to about 50% (w/w) anti-TSLP antibody or anti-TSLP antibody fragment, about 10% (w/w) to about 15% (w/w) trileucine, about 25% (w/w) to about 85% (w/w) trehalose, and a buffer.

In some embodiments, the pharmaceutical composition of the invention comprises:
a) about 2% (w/w) to about 15% (w/w) anti-TSLP antibody or anti-TSLP antibody fragment, about 10% (w/w) to about 15% (w/w) trileucine, about 65% (w/w) to about 85% (w/w) trehalose, and a buffer;
b) about 10% (w/w) to about 25% (w/w) anti-TSLP antibody or anti-TSLP antibody fragment, about 10% (w/w) to about 15% (w/w) trileucine, about 50% (w/w) to about 75% (w/w) trehalose, and a buffer;
c) about 20% (w/w) to about 25% (w/w) anti-TSLP antibody or anti-TSLP antibody fragment, about 10% (w/w) to about 15% (w/w) trileucine, about 50% (w/w) to about 60% (w/w) trehalose, and a buffer;
d) about 20% (w/w) to about 40% (w/w) anti-TSLP antibody or anti-TSLP antibody fragment, about 10% (w/w) to about 15% (w/w) trileucine, about 35% (w/w) to about 60% (w/w) trehalose, and a buffer;
e) about 40% (w/w) to about 50% (w/w) anti-TSLP antibody or anti-TSLP antibody fragment, about 10% (w/w) to about 15% (w/w) trileucine, about 25% (w/w) to about 35% (w/w) trehalose, and a buffer.

In some embodiments, the pharmaceutical composition of the invention comprises:
a) about 3% (w/w) anti-TSLP antibody or anti-TSLP antibody fragment, about 10% (w/w) trileucine, about 81% (w/w) trehalose, and a buffer;
b) about 5% (w/w) anti-TSLP antibody or anti-TSLP antibody fragment, about 15% (w/w) trileucine, about 71% (w/w) trehalose, and a buffer;
c) about 6% (w/w) anti-TSLP antibody or anti-TSLP antibody fragment, about 10% (w/w) trileucine, about 78% (w/w) trehalose, and a buffer;
d) about 10% (w/w) anti-TSLP antibody or anti-TSLP antibody fragment, about 15% (w/w) trileucine, about 66% (w/w) trehalose, and a buffer;
e) about 10% (w/w) anti-TSLP antibody or anti-TSLP antibody fragment, about 10% (w/w) trileucine, about 74% (w/w) trehalose, and a buffer;
f) about 12.5% (w/w) anti-TSLP antibody or anti-TSLP antibody fragment, about 10% (w/w) trileucine, about 71% (w/w) trehalose, and a buffer;
g) about 20% (w/w) anti-TSLP antibody or anti-TSLP antibody fragment, about 15% (w/w) trileucine, about 55.5% (w/w) trehalose, and a buffer;
h) about 25% (w/w) anti-TSLP antibody or anti-TSLP antibody fragment, about 15% (w/w) trileucine, about 50% (w/w) trehalose, and a buffer;
i) about 25% (w/w) anti-TSLP antibody or anti-TSLP antibody fragment, about 10% (w/w) trileucine, about 58% (w/w) trehalose, and a buffer;
j) about 40% (w/w) anti-TSLP antibody or anti-TSLP antibody fragment, about 15% (w/w) trileucine, about 35% (w/w) trehalose, and a buffer;
k) about 50% (w/w) anti-TSLP antibody or anti-TSLP antibody fragment, about 15% (w/w) trileucine, about 24.5% (w/w) trehalose, and a buffer;
l) about 50% (w/w) anti-TSLP antibody or anti-TSLP antibody fragment, about 10% (w/w) trileucine, about 32.5% (w/w) trehalose, and a buffer.

In some embodiments, the pharmaceutical composition of the invention comprises:
a) 3.13% (w/w) anti-TSLP antibody or anti-TSLP antibody fragment, 10% (w/w) trileucine, 80.83% (w/w) trehalose, and a buffer;
b) 5% (w/w) anti-TSLP antibody or anti-TSLP antibody fragment, 15% (w/w) trileucine, 70.91% (w/w) trehalose, and a buffer;
c) 6.25% (w/w) anti-TSLP antibody or anti-TSLP antibody fragment, 10% (w/w) trileucine, 77.61% (w/w) trehalose, and a buffer;
d) 10% (w/w) anti-TSLP antibody or anti-TSLP antibody fragment, 15% (w/w) trileucine, 65.76% (w/w) trehalose, and a buffer;
e) 10% (w/w) anti-TSLP antibody or anti-TSLP antibody fragment, 10% (w/w) trileucine, 73.73% (w/w) trehalose, and a buffer;
f) 12.5% (w/w) anti-TSLP antibody or anti-TSLP antibody fragment, 10% (w/w) trileucine, 71.15% (w/w) trehalose, and a buffer;
g) 20% (w/w) anti-TSLP antibody or anti-TSLP antibody fragment, 15% (w/w) trileucine, 55.44% (w/w) trehalose, and a buffer;
h) 25% (w/w) anti-TSLP antibody or anti-TSLP antibody fragment, 15% (w/w) trileucine, 50.29% (w/w) trehalose, and a buffer;
i) 25% (w/w) anti-TSLP antibody or anti-TSLP antibody fragment, 10% (w/w) trileucine, 58.26% (w/w) trehalose, and a buffer;
j) 40% (w/w) anti-TSLP antibody or anti-TSLP antibody fragment, 15% (w/w) trileucine, 34.81% (w/w) trehalose, and a buffer;
k) 50% (w/w) anti-TSLP antibody or anti-TSLP antibody fragment, 15% (w/w) trileucine, 24.5% (w/w) trehalose, and a buffer;
l) 50% (w/w) anti-TSLP antibody or anti-TSLP antibody fragment, 10% (w/w) trileucine, about 32.48% (w/w) trehalose, and a buffer.

In some embodiments, the pharmaceutical composition of the invention comprises about 2% (w/w) to about 50% (w/w) anti-TSLP antibody or anti-TSLP antibody fragment, in particular about 3% (w/w) to about 50% (w/w) anti-TSLP antibody or anti-TSLP antibody fragment, about 10% (w/w) to about 15% (w/w) trileucine, about 25% (w/w) to about 85% (w/w) trehalose, about 5% (w/w) to about 10% (w/w) histidine, and about 1% (w/w) to about 2% (w/w) HCl.

In some embodiments, the pharmaceutical composition of the invention comprises:
a) about 2% (w/w) to about 15% (w/w) anti-TSLP antibody or anti-TSLP antibody fragment, about 10% (w/w) to about 15% (w/w) trileucine, about 65% (w/w) to about 85% (w/w) trehalose, about 5% (w/w) to about 10% (w/w) histidine, and about 1% (w/w) to about 2% (w/w) HCl;
b) about 10% (w/w) to about 25% (w/w) anti-TSLP antibody or anti-TSLP antibody fragment, about 10% (w/w) to about 15% (w/w) trileucine, about 50% (w/w) to about 75% (w/w) trehalose, about 5% (w/w) to about 10% (w/w) histidine, and about 1% (w/w) to about 2% (w/w) HCl;
c) about 20% (w/w) to about 25% (w/w) anti-TSLP antibody or anti-TSLP antibody fragment, about 10% (w/w) to about 15% (w/w) trileucine, about 50% (w/w) to about 60% (w/w) trehalose, about 5% (w/w) to about 10% (w/w) histidine, and about 1% (w/w) to about 2% (w/w) HCl;
d) about 20% (w/w) to about 40% (w/w) anti-TSLP antibody or anti-TSLP antibody fragment, about 10% (w/w) to about 15% (w/w) trileucine, about 35% (w/w) to about 60% (w/w) trehalose, about 5% (w/w) to about 10% (w/w) histidine, and about 1% (w/w) to about 2% (w/w) HCl;
e) about 40% (w/w) to about 50% (w/w) anti-TSLP antibody or anti-TSLP antibody fragment, about 10% (w/w) to about 15% (w/w) trileucine, about 25% (w/w) to about 35% (w/w) trehalose, about 5% (w/w) to about 10% (w/w) histidine, and about 1% (w/w) to about 2% (w/w) HCl.

In some more specific embodiments, the pharmaceutical composition of the invention comprises:
a) about 3% (w/w) anti-TSLP antibody or anti-TSLP antibody fragment, about 10% (w/w) trileucine, about 81% (w/w) trehalose, about 5% (w/w) histidine, and about 1% (w/w) HCl;
b) about 5% (w/w) anti-TSLP antibody or anti-TSLP antibody fragment, about 15% (w/w) trileucine, about 71% (w/w) trehalose, about 7.5% (w/w) histidine, and about 1.5% (w/w) HCl;
c) about 6% (w/w) anti-TSLP antibody or anti-TSLP antibody fragment, about 10% (w/w) trileucine, about 78% (w/w) trehalose, about 5% (w/w) histidine, and about 1% (w/w) HCl;
d) about 10% (w/w) anti-TSLP antibody or anti-TSLP antibody fragment, about 15% (w/w) trileucine, about 66% (w/w) trehalose, about 7.5% (w/w) histidine, and about 1.5% (w/w) HCl;
e) about 10% (w/w) anti-TSLP antibody or anti-TSLP antibody fragment, about 10% (w/w) trileucine, about 74% (w/w) trehalose, about 5% (w/w) histidine, and about 1% (w/w) HCl;
f) about 12.5% (w/w) anti-TSLP antibody or anti-TSLP antibody fragment, about 10% (w/w) trileucine, about 71% (w/w) trehalose, about 5% (w/w) histidine, and about 1% (w/w) HCl;
g) about 20% (w/w) anti-TSLP antibody or anti-TSLP antibody fragment, about 15% (w/w) trileucine, about 55.5% (w/w) trehalose, about 8% (w/w) histidine, and about 1.5% (w/w) HCl;
h) about 25% (w/w) anti-TSLP antibody or anti-TSLP antibody fragment, about 15% (w/w) trileucine, about 50% (w/w) trehalose, about 8% (w/w) histidine, and about 2% (w/w) HCl;
i) about 25% (w/w) anti-TSLP antibody or anti-TSLP antibody fragment, about 10% (w/w) trileucine, about 58% (w/w) trehalose, about 6% (w/w) histidine, and about 1% (w/w) HCl;
j) about 40% (w/w) anti-TSLP antibody or anti-TSLP antibody fragment, about 15% (w/w) trileucine, about 35% (w/w) trehalose, about 8% (w/w) histidine, and about 2% (w/w) HCl;
k) about 50% (w/w) anti-TSLP antibody or anti-TSLP antibody fragment, about 15% (w/w) trileucine, about 24.5% (w/w) trehalose, about 8.5% (w/w) histidine, and about 2% (w/w) HCl;
l) about 50% (w/w) anti-TSLP antibody or anti-TSLP antibody fragment, about 10% (w/w) trileucine, about 32.5% (w/w) trehalose, about 6% (w/w) histidine, and about 1.5% (w/w) HCl.

In some even more specific embodiments, the pharmaceutical composition of the invention comprises:
a) 3.13% (w/w) anti-TSLP antibody or anti-TSLP antibody fragment, 10% (w/w) trileucine, 80.83% (w/w) trehalose, 5.01% (w/w) histidine, and 1.04% (w/w) HCl;
b) 5% (w/w) anti-TSLP antibody or anti-TSLP antibody fragment, 15% (w/w) trileucine, 70.91% (w/w) trehalose, 7.53% (w/w) histidine, and 1.56% (w/w) HCl;
c) 6.25% (w/w) anti-TSLP antibody or anti-TSLP antibody fragment, 10% (w/w) trileucine, 77.61% (w/w) trehalose, 5.09% (w/w) histidine, and 1.05% (w/w) HCl;
d) 10% (w/w) anti-TSLP antibody or anti-TSLP antibody fragment, 15% (w/w) trileucine, 65.76% (w/w) trehalose, 7.66% (w/w) histidine, and 1.59% (w/w) HCl;
e) 10% (w/w) anti-TSLP antibody or anti-TSLP antibody fragment, 10% (w/w) trileucine, 73.73% (w/w) trehalose, 5.19% (w/w) histidine, and about 1.08% (w/w) HCl;
f) 12.5% (w/w) anti-TSLP antibody or anti-TSLP antibody fragment, 10% (w/w) trileucine, 71.15% (w/w) trehalose, 5.25% (w/w) histidine, and 1.09% (w/w) HCl;
g) 20% (w/w) anti-TSLP antibody or anti-TSLP antibody fragment, 15% (w/w) trileucine, 55.44% (w/w) trehalose, 7.92% (w/w) histidine, and 1.64% (w/w) HCl;
h) 25% (w/w) anti-TSLP antibody or anti-TSLP antibody fragment, 15% (w/w) trileucine, 50.29% (w/w) trehalose, 8.05% (w/w) histidine, and 1.67% (w/w) HCl;
i) 25% (w/w) anti-TSLP antibody or anti-TSLP antibody fragment, 10% (w/w) trileucine, 58.26% (w/w) trehalose, 5.58% (w/w) histidine, and 1.16% (w/w) HCl;
j) 40% (w/w) anti-TSLP antibody or anti-TSLP antibody fragment, 15% (w/w) trileucine, 34.81% (w/w) trehalose, 8.43% (w/w) histidine, and 1.75% (w/w) HCl;
k) 50% (w/w) anti-TSLP antibody or anti-TSLP antibody fragment, 15% (w/w) trileucine, 24.5% (w/w) trehalose, 8.69% (w/w) histidine, and 1.81% (w/w) HCl;
l) 50% (w/w) anti-TSLP antibody or anti-TSLP antibody fragment, 10% (w/w) trileucine, about 32.48% (w/w) trehalose, 6.22% (w/w) histidine, and 1.3% (w/w) HCl.

In some embodiments, the pharmaceutical composition of the present invention can be prepared as a capsule or a blister or a blister package, which is suitable for administration through inhalation, in particular wherein the capsule or the blister comprises from about 0.5 mg to about 16 mg, e.g., about 1 mg to about 16 mg, about 2 mg to about 16 mg, about 2 mg to about 8 mg, in particular about 4 mg to about 8 mg, of the anti-TSLP antibody or anti-TSLP antibody fragment. Suitably, the capsule or the blister comprises from 0.5 mg to 16 mg, e.g., 1 mg to 16 mg, 2 mg to 16 mg, 2 mg to 8 mg, in particular 4 mg to 8 mg, of the anti-TSLP antibody or anti-TSLP antibody fragment. Suitably, the capsule or the blister comprises about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg or about 16 mg, of the anti-TSLP antibody or anti-TSLP antibody fragment. Suitably, the capsule or the blister comprises 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg or 16 mg, of the anti-TSLP antibody or anti-TSLP antibody fragment.

In a further aspect, the present invention relates to a medicament for administration through inhalation comprising the anti-TSLP antibody or anti-TSLP antibody fragment of the disclosure, in particular the anti-TSLP antibody fragment of the disclosure, more particularly the anti-TSLP antibody Fab of the disclosure, wherein the medicament comprises about 0.5 mg to about 16 mg, e.g., about 1 mg to about 16 mg, about 2 mg to about 16 mg, about 2 mg to about 8 mg, in particular about 4 mg to about 8 mg. Suitably, the medicament comprises 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg or 16 mg, of said anti-TSLP antibody or anti-TSLP antibody fragment.

In more specific embodiments, the anti-TSLP antibody or anti-TSLP antibody fragment of the medicament of the invention comprises
a) a heavy chain comprising
   i. the amino acid sequence of SEQ ID NO: 9, or
   ii. a sequence at least 90% identical to SEQ ID NO: 9, or
   iii. a conservative variant of SEQ ID NO: 9, and
b) a light chain comprising
   i. the amino acid sequence of SEQ ID NO: 19, or
   ii. a sequence at least 90% identical to SEQ ID NO: 19, or
   iii. a conservative variant of SEQ ID NO: 19.

In some embodiments, the anti-TSLP antibody or anti-TSLP antibody fragment of the medicament of the invention is selected from the group consisting of a human antibody, humanized antibody, a chimeric antibody, a monoclonal antibody, a recombinant antibody. In some embodiments, the anti-TSLP antibody fragment is utilized in the medicament of the invention and is selected from the group consisting of a Fab, Fab′, F(ab′)2, scFv, minibody, or diabody, in particular wherein the antibody fragment is a Fab, in particular human or humanized Fab. In some embodiments, the anti-TSLP antibody of the medicament of the invention is a human immunoglobulin.

In one embodiment, the medicament is in a powder form, in particular in a dry powder form. In a further embodiment, the medicament is in powder form stored in a capsule or in a blister (which is suitable for administration via inhalation).

In a further aspect, the present invention provides a kit comprising the pharmaceutical composition of the invention or the medicament of the invention, and a device for delivering the pharmaceutical composition or the medicament to a subject. In some embodiments, the device utilized in the kit delivers the pharmaceutical composition or the medicament in an aerosolized form. In some embodiments, the device is a dry powder inhaler.

In a further aspect, the present invention provides the pharmaceutical composition of the invention or the medicament of the invention for use in the treatment of an inflammatory or obstructive airway disease. In some embodiments, the present invention provides the pharmaceutical composition of the invention or the medicament of the invention for use in the treatment of an inflammatory or obstructive airway disease according to the methods described herein, in particular by administering to a subject a dose of about 0.5 mg to about 16 mg of an anti-TSLP antibody or anti-TSLP antibody fragment.

### Abbreviations

ACQ-5 Asthma Control Questionnaire-5
AE adverse event
AIC allergen inhalation challenge
ANCOVA analysis of covariance
AUC area under the curve
AUCinf area under the plasma (or serum or blood) concentration-time curve from time zero to infinity (mass x time/volume)
AUClast area under the plasma (or serum or blood) concentration-time curve from time zero to time of last quantifiable concentration (mass x time/volume)
AUCtau area under the plasma (or serum or blood) concentration-time curve from time zero to the end of the dosing interval tau (mass x time/volume)
b.i.d. twice a day
CL/F the apparent systemic (or total body) clearance from plasma (or serum or blood) following administration (mass/volume)
Cmax Maximum concentration observed in concentration/time profile
COA Clinical Outcome Assessments
DPI Dry Powder Inhaler
ECG Electrocardiogram
EOT end of treatment
FEF forced expiratory flow
FeNO fractional nitric oxide measurements
FEV1 Forced Expiratory Volume in 1 second
FVC forced vital capacity
h hour
IA interim analysis
ICS Inhaled Corticosteroids
LABA Long-acting β2 agonist
LAMA Long-acting muscarinic antagonist
LAR late asthmatic response
LLOQ lower limit of quantification
LTRA Leukotriene receptor antagonist
mg milligram(s)
MIC methacholine inhalation challenge
mL milliliter(s)
NOAEL no observed adverse effect level
PD pharmacodynamic(s)
PEF peak expiratory flow rate
PK pharmacokinetic(s)
q.d. (QD) once daily
Racc ratio of accumulation of drug
SAD single ascending dose
SABA short-acting beta2-agonist
SAE serious adverse event
SD standard deviation
T½ Half-life
Tmax Time of maximum concentration observed in concentration/time profile
TSLP Thymic stroma lymphopoietin
Vz/F the apparent volume of distribution during the terminal elimination phase following administration (volume)

The following Examples illustrates the invention described above, but is not, however, intended to limit the scope of the invention in any way. Other test models known as such to the person skilled in the pertinent art can also determine the beneficial effects of the claimed invention.

### EXAMPLES

### Example 1: Phase 1 Clinical Trial: The Safety, Tolerability and PK of CSJ117 in Healthy Volunteers

The safety, tolerability and PK of CSJ117 have been evaluated in healthy volunteers (HV) in a single ascending dose (SAD) study.

A first-in-human, randomized, subject-blind, placebo controlled, single ascending dose study was conducted to assess the safety, tolerability and pharmacokinetics of CSJ117 in adult healthy subjects. Single dose of 1 mg (Cohort 1), 3 mg (Cohort 2), 9 mg (Cohort 3), 32 mg (Cohort 4), 64 mg (Cohort 5), 160 mg (Cohort 6) or placebo was administered via inhalation. First-in-human study was conducted in 51 healthy volunteers (HVs), of which 37 subjects were exposed to CSJ117 and 12 to placebo.

The PK analysis set for the FIH SAD Study included 37 subjects. PK samples were collected up to 28 days post dose. A number of subjects at the low doses did not have detectable CSJ117 in serum (4 out of 7 in Cohort 1 and 1 out of 6 in Cohort 2). Serum CSJ117 concentrations above the LLOQ (3 ng/mL) were detected up to 72 hours post dose for Cohort 3 (9 mg) and up to 96 hours post-dose for Cohorts 4-6 (32, 64 and 160 mg).

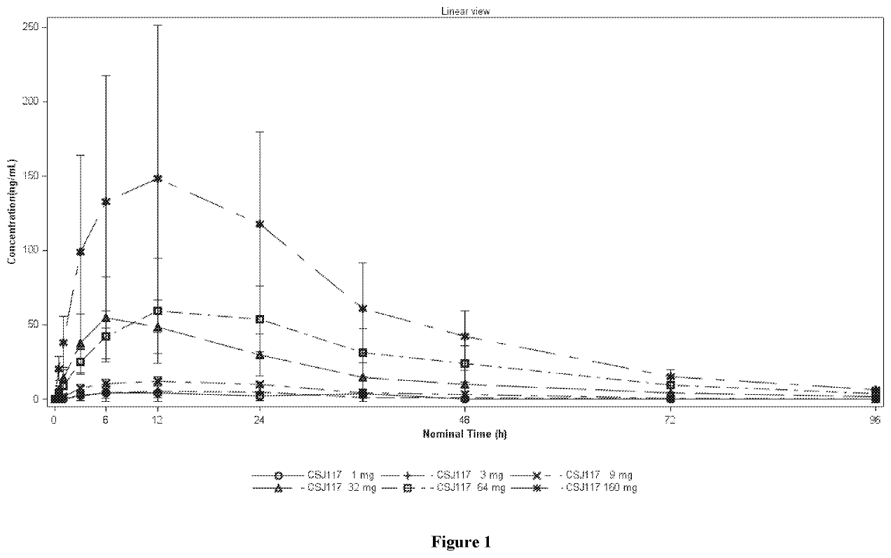

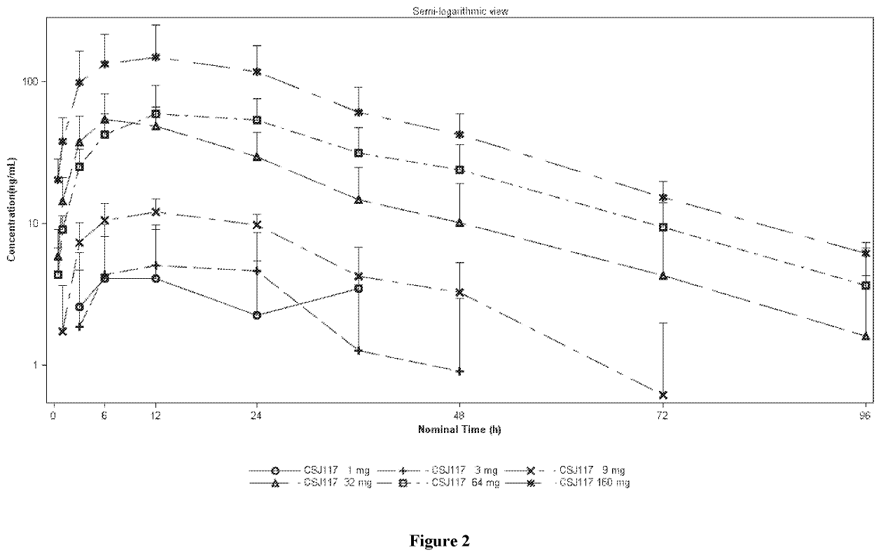

Serum concentration-time profiles are presented in FIGS. 1 and 2 and summary of PK parameters is presented in Table 4. CSJ117 serum exposure increased in a dose-dependent manner in the dose range of 1-160 mg following single inhaled doses of CSJ117 via the Concept1 device. The median time to achieve maximum CSJ117 serum concentration (Tmax) ranged from 6 to 21 hours and the mean terminal elimination half-life (T½) ranged from 17.4 to 21.1 hours.

In the FIH study, there were no deaths or serious adverse events (SAEs), and there were no discontinuations due to adverse events. CSJ117 subjects did not experience a greater number of AEs (7/37, 19% of subjects) compared to placebo (5/12, 42% of subjects) and all of the AEs were mild except for two moderate AEs: (1) toothache requiring extraction in the 160 mg cohort on day 16, and (2) headache in the placebo group on day 1.

In addition, the PK analysis set for the healthy Japanese SAD Study included 18 subjects, six subjects per single inhaled dose of 9, 16 and 32 mg administered via Concept 1 device. CSJ117 serum exposure increased in a dose-dependent manner in the examined dose range; however, less than dose proportional increase in exposure was observed, possibly due to limited sample size. Median Tmax ranged from 9 to 12 hours and mean T1/2 ranged from 18.6 to 22.4 hours. Cross-study comparison of the exposures at the 9 and 32 mg doses from first in human study showed Cmax and AUCinf in Japanese within the range of 0.5-1.1-fold (Cmax) and 0.6-1.5-fold (AUCinf) of non-Japanese subjects. The data suggested there was no major impact of ethnic differences on PK properties of CSJ117.

TABLE 4
Summary of serum CSJ117 pharmacokinetic parameters following single
dose inhaled administration.

| | | Tmax* | Cmax | AUClast | AUCinf | T1/2 | Vz/F | CL/F |
|---|---|---|---|---|---|---|---|---|
| Dose | Parameter | (hr) | (ng/mL) | (ng.hr/mL) | (ng.hr/mL) | (hr) | (L) | (L/hr) |
| 1 mg** | N | 2 | 2 | 2 | 0 | 1 | 0 | 0 |
| | Mean | 21.0 | 5.90 | 109 | ND | 18.5 | ND | ND |
| | | (6.00, | | | | | | |
| | | 36.0) | | | | | | |
| 3 mg | N | 5 | 5 | 5 | 0 | 1 | 0 | 0 |
| | Mean | 12.0 | 5.65 | 127 | ND | 18.7 | ND | ND |
| | (CV %) | (12.0, | (58.1) | (124) | | | | |
| | | 24.0) | | | | | | |
| 9 mg | N | 6 | 6 | 6 | 2 | 3 | 2 | 2 |
| | Mean | 12.0 | 12.5 | 353 | 597 | 21.1 | 481 | 15.6 |
| | (CV %) | (6.00, | (21.2) | (39) | | (42.0) | | |
| | | 24.0) | | | | | | |
| 32 mg | N | 6 | 6 | 6 | 6 | 6 | 6 | 6 |
| | Mean | 6.00 | 57.6 | 1610 | 1710 | 17.4 | 555 | 22.0 |
| | (CV %) | (6.00, | (47.1) | (41.1) | (40.9) | (43.8) | (79.9) | (47.3) |
| | | 24.0) | | | | | | |
| 64 mg | N | 6 | 6 | 6 | 6 | 6 | 6 | 6 |
| | Mean | 12.0 | 61.4 | 2500 | 2650 | 20.1 | 815 | 28.5 |
| | (CV %) | (12.0, | (55.8) | (49.6) | (48.2) | (10.6) | (37.9) | (40.9) |
| | | 24.0) | | | | | | |
| 160 mg | N | 6 | 6 | 6 | 6 | 6 | 6 | 6 |
| | Mean | 9.00 | 152 | 5590 | 5750 | 18.2 | 915 | 33.0 |
| | (CV %) | (5.92, | (68.0) | (53.5) | (51.5) | (20.9) | (58.5) | (39.9) |
| | | 12.0) | | | | | | |

*Median and range given for Tmax; ND: not determined, % CV not reported when N < 3
**Subject 1001045 (1 mg) excluded from analysis as an outlier [unexpected pre-dose serum concentration (13.9 ng/mL) and similar concentrations (range: 10.9-14.4 ng/mL) throughout the study duration up to 168 hours post-dose, anti-CSJ117 antibodies not detected].

### Example 2: Phase 2a Clinical Trial: A Randomized, Subject- and Investigator-Blinded, Placebo-Controlled, Parallel-Design, Bronchoprovocation Study to Evaluate the Safety, Tolerability, Pharmacokinetics and Pharmacodynamics of Multiple Doses of Inhaled CSJ117 in Adult Subjects with Mild Atopic Asthma

CSJ117, as an inhaled Fab targeting TSLP, offers the potential to be an efficacious therapy for severe asthma with a favorable safety and tolerability profile. This study used inhaled allergen bronchoprovocation testing as a model for allergic asthma, in an effort to predict CSJ117 efficacy in the eventual severe asthma patient population. As a model for allergic asthma, standardized inhaled allergen bronchoprovocation testing can induce the acute and some of the more chronic features of asthma in human subjects with mild atopic asthma. In sensitized subjects, inhalation of a relevant allergen results in acute bronchoconstriction (the early asthma response or EAR [EAR defined as ≥20% fall in forced expiratory volume in 1 second (FEV1) between 0-2 hours after inhalation of allergen] Boulet et al 2007). In approximately 50% of subjects the EAR is followed by an inflammatory airway response with prolonged airway narrowing (the late asthma response or LAR [LAR defined as ≥15% fall in FEV1 between 3-7 hours after inhalation of allergen] Boulet et al 2007). In general, inhibition of the LAR in the allergen bronchoprovocation model is a good predictor of clinical efficacy in the moderate to severe asthma patient population (Diamant et al 2013). The objectives of this allergen bronchoprovocation study were to characterize the safety, tolerability, pharmacokinetics (PK) and pharmacodynamic (PD) effects of CSJ117 in subjects with mild atopic asthma over 12 weeks of treatment to determine if further clinical development is warranted.

### Primary Objective(s)

| Objective | Endpoint |
|---|---|
| To evaluate the safety and tolerability | Adverse events (AEs) and serious adverse events |
| of CSJ117 over 12 weeks in subjects | (SAEs). |
| with mild atopic asthma. | |
| To evaluate the late asthmatic | Area under the curve (AUC) for the time-adjusted |
| response (LAR) after an allergen | percent fall and maximum percentage fall in the |
| inhalation challenge (AIC) on Day 84 | FEVI between 3 and 7 hours after the |
| in subjects with mild atopic asthma | administration of the AIC on Day 84. |
| receiving multiple doses of CSJ117 | |
| over 12 weeks. | |

Secondary objective(s)

| | |
|---|---|
| To evaluate the early asthma response | The time-adjusted AUC, maximum |
| (EAR) and additional measures of the | percentage fall and the minimum of the |
| LAR after an AIC on Day 42 and Day | absolute FEV1 between 0 and 2 hours |
| 84 in subjects with mild atopic asthma | (EAR) after the administration of the AIC |
| receiving multiple doses of CSJ117 | (Day 42 and Day 84). |
| over 12 weeks. | The time adjusted AUC (Day 42), |
| | maximum percentage fall (Day 42) and the |
| | minimum absolute FEV1 (Day 42 and 84) |
| | between 3 and 7 hours (LAR) after the |
| | administration of the AIC. |
| To characterize the systemic PK | Measurement of total CSJ117 serum |
| profile of CSJ117 in subjects with | concentration and calculation of PK |
| mild atopic asthma after | parameters (e.g., Tmax, Cmax, AUCtau, |
| administration of multiple inhaled | Ctrough, Race, Lambda_z, T½). |
| doses. | Other PK parameters may be derived based |
| | on the PK data obtained. |
| To evaluate additional measures of the | Safety assessments, including vital signs, |
| safety and tolerability of multiple dose | laboratory evaluations, ECG and spirometry. |
| administration of CSJ117 over 12 | |
| weeks in subjects with mild atopic | |
| asthma. | |
| To evaluate the immunogenicity of | Measurement of anti-CSJ117 antibodies (anti-drug |
| multiple dose administration of | antibodies; ADA). |
| CSJ117 over 12 weeks in subjects | |
| with mild atopic asthma. | |

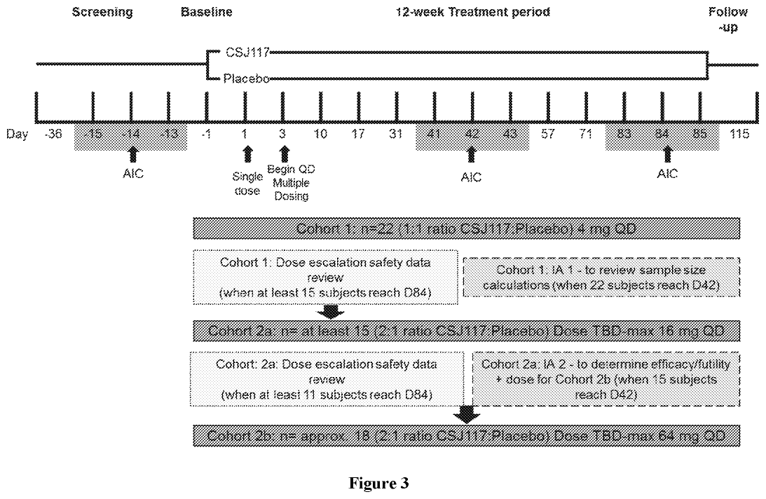

### Study Design (FIG. 3):

This was a non-confirmatory, randomized, subject- and investigator-blinded, placebo-controlled, parallel-design, multi-center bronchoprovocation study. Adult subjects with stable mild atopic asthma who exhibited an EAR and LAR to a common inhaled allergen were randomized to receive 4 mg QD of inhaled CSJ117 or placebo over 12 weeks and the following key parameters were assessed:
The safety and tolerability of CSJ117 by evaluation of AEs, SAEs and other safety parameters.
The PK profile of CSJ117 by measurement of total serum concentrations.
The PD response to CSJ117 by analysis of the LAR following AIC after a 12-week treatment period.
   Two sequential dose cohorts were enrolled for this study.
Cohort 1: 24 subjects (12 active and 12 placebo)
Cohort 2a: 4 subjects (3 active and 1 placebo)

There was only one active dose used in the study, i.e. CSJ117 4 mg used for both Cohort 1 & 2a. Placebo subjects from all cohorts were pooled for analyses.

For all cohorts, the study consisted of a 35-day screening period, a baseline evaluation, a 12-week treatment period (consisting of a single dose on Day 1, safety evaluation Days 1-3 and commencement of QD dosing on Day 3) and a 30-day follow up period (FIG. 3).

After providing informed consent and an initial screening period from Day-29 to Day-16, subjects who met all eligibility criteria went for further screening assessments (methacholine inhalation challenges [MIC], induced-sputum sample collections, fractional nitric oxide measurements [FeNO] and AIC) over 3 Days (Day −15, −14 and −13).

Subjects who qualified based on all screening assessments returned for a baseline evaluation on Day −1. Subjects who met all the applicable inclusion/exclusion criteria at screening and baseline continued to the treatment period of the study.

On Day 1 (Visit 101) of the treatment period, after completion of all pre-dose assessments, subjects were randomized and received a single dose of CSJ117 or placebo in the morning and then underwent safety assessments, observation and PK sampling in-clinic for a period of 8 hours post-dose. On Day 2 (Visit 102), the subject returned to the clinic for evaluation and PK sampling. On Day 3 (Visit 103), the subjects returned to clinic for evaluation. On Day 3 (Visit 103) subjects took an inhaled dose of CSJ117 or placebo in the clinic. For the remaining duration of the 12-week treatment period, subjects took daily inhaled doses of CSJ117 or placebo, in the clinic on study visit days or at home. Subjects completed dosing on Day 84 and complete an end-of-treatment (EOT) period visit on Day 85.

Two interim analysis (IAs) were conducted for this study. The first interim analysis was conducted to review the sample size of the study. The second interim analysis was carried out to evaluate the safety and efficacy of CSJ117. IA1: 20 subjects from Cohort 1 (10 in CSJ117 4 mg group and 10 in Placebo group). IA2: 28 subjects from Cohort 1 & 2a (15 in CSJ117 4 mg group and 13 in Placebo group)

### Rationale for Dose Regimen, Route of Administration and Duration of Treatment:

CSJ117 was designed for inhaled delivery. CSJ117 is formulated as a PulmoSol™ engineered powder in hard capsules for delivery to the lungs via dry powder inhaler (DPI; Concept1).

The rationale for a 12-week study treatment duration was based on the following: (i) the 12-week treatment period was warranted from a safety perspective given the data from the 13-week cynomolgus monkey toxicity study; (ii) the 12-week dosing period was anticipated to provide greater information on the PK profile of CSJ117 in humans (as compared to a shorter study duration); (iii) completing 12-weeks of dosing prior to dose escalation(s) provided greater safety information, as systemic exposure is expected to have achieved steady state; (iv) 12 weeks was expected to be sufficient duration to allow assessment of PD effects in the AIC.

The proposed dose for the bronchoprovocation study was 4 mg/day, based on clinical and nonclinical safety data:
The 4 mg QD dose was at the lower end of the dose range safely tested in the single ascending dose (SAD) healthy volunteer study (Example 1) in which 6 cohorts, up to a dose level of 160 mg, completed.
Based on the nonclinical safety data in the GLP monkey toxicity study, the 4 mg QD dose was below the human equivalent dose of the monkey NOAEL (based on lung weight scaling) with a 51.5-fold dose multiple. Lung weight scaling was selected for the HED calculation due to the CSJ117-related changes in the respiratory tract and the lack of significant systemic side effects in the monkey toxicity studies.
Exposure multiples were also calculated to compare the observed systemic exposure in monkeys with the systemic exposure in humans, based on the observed single dose PK data in healthy subjects, assuming either linear or nonlinear PK. For multiple daily inhalations in humans, assuming linear and time-independent PK (i.e., a 2.4 fold accumulation ratio), the estimated human systemic exposure at steady state for a 4 mg dose was 948-fold below the monkey Day 91 systemic exposure. Alternatively, if nonlinear and time-dependent PK was assumed, and if the systemic accumulation observed in monkeys translates directly to humans following multiple daily inhalations (i.e., a 148-fold accumulation ratio), the human systemic exposure at steady state for a 4 mg dose was 15.9-fold below the monkey Day 91 systemic exposure.

Based on predictions of the CSJ117 clinical efficacious dose range and PK profile after repeated administration, once daily dosing was expected to provide adequate pulmonary exposure in order to assess PD effects.

### Population:

The study population was comprised of male and female subjects with mild atopic asthma between the ages of 18 to 60.

### Key Inclusion Criteria:

Patients with mild atopic asthma who were eligible for inclusion in this study fulfilled all of the following key criteria:

1. Written informed consent was obtained before any assessment is performed.

2. Able to communicate well with the site Investigator, to understand and comply with the requirements of the study.

3. Otherwise healthy (see inclusion criteria 7) male and female subjects 18 to 60 years of age, as determined by past medical history, physical examination, vital signs and ECG at screening and baseline (Visits 1 and 5) and laboratory tests at screening (Visit 1).

4. Screening (Visit 1) FEV1>70% of the predicted normal value after a wash out of at least 8 hours for inhaled short-acting bronchodilators.

5. Diagnosis of stable mild atopic asthma, as defined by the American Thoracic Society/European Respiratory Society statement (Reddel et al 2009) who exhibited an early and late asthmatic response to a common inhaled allergen during the screening AIC. Only subjects who required infrequent use of inhaled short acting beta-2 agonists (<twice weekly) for asthma symptoms were eligible for inclusion.

6. Airway hyper-responsiveness documented by a provocative concentration of methacholine causing a >20% fall in FEV1 (PC20 FEV1) of less than or equal to 16 mg/ml during the screening MIC (Visit 2).

7. Positive skin prick test to common aeroallergens at screening (Visit 1).

8. Throughout the screening period and at baseline, only infrequent use of inhaled short-acting β2-agonists (<twice weekly) to treat asthma or prophylactic use prior to exercise. Inhaled short-acting β2-agonist was withheld for 8 hours before spirometry.

Key Exclusion Criteria:

Patients with mild atopic asthma fulfilling any of the following key criteria were not eligible for inclusion in this study:

1. Hospitalization or emergency room treatment for acute asthma in the 6 months prior to screening (Visit 1) or during the entire screening period.

2. Intubation (ever) or hospitalization for longer than 24 hours for the management of an asthma exacerbation within the preceding 6 months of screening (Visit 1).

3. Any worsening or exacerbation of asthma (e.g., an event requiring a change in treatment) in the six weeks before screening (Visit 1) or during the entire screening period.

4. Subjects who required any concomitant or prohibited medications for asthma control were not eligible for inclusion. If these medications were required for other indications (e.g. allergic rhinitis), these medications were appropriately washed out prior to Day −15.

5. Current exposure to allergens to which a subject experiences asthmatic responses (with the exception of the house dust mite) at screening (Visit 1).

6. A history or current medical conditions that were contraindicated for allergy skin tests, including a history of severe anaphylactic reactions, skin disease or eczema which could interact with skin test results, mastocytosis or treatment with a beta-blocker.

7. History or current medical conditions that were contraindicated for MIC and AIC, such as myocardial infarction or stroke within the previous 3 months to screening (Visit1), known cardiac disease including unstable coronary perfusion or uncontrolled hypertension, and aortic or cerebral aneurysm.

8. A history of any clinically significant chronic pulmonary disease other than mild atopic asthma, including but not limited to COPD, interstitial lung disease or bronchiectasis.

9. Evidence of any active or suspected clinically significant infection in the 6 weeks prior to screening (Visit 1) or during the entire screening period. This included any upper respiratory tract infection (e.g., common cold, influenza, sinusitis) or pulmonary infection (e.g., pneumonia). Any subject, who in the opinion of the site Investigator had a high risk of parasitic disease, was also excluded.

10. Use of all over-the-counter or prescription medications (unless otherwise specified in this protocol, such as inhaled short-acting 02-agonists), herbal medicines and vitamins/supplements within 14 days or 5 half-lives (whichever is longer) prior to Day 1 (Visit 101) was reviewed by Novartis to confirm whether a subject can be randomized into the study.

### Results:

A total of 28 subjects were enrolled in the study, out of which 15 received CSJ117 and 13 received placebo. One subject in the CSJ117 treatment group discontinued from the study due to a the physician's decision by her physician. The median age was 32.5 years. Seventeen subjects (61%) were female. Twenty-four subjects were Caucasian and three subjects were Hispanic. Baseline disease characteristics were similar between the CSJ117 and the placebo group (Table 5), with no clinically relevant differences noted. All subjects had stable mild atopic asthma.

TABLE 5
Baseline characteristics.

| | | CSJ117 4 mg | Placebo | Total |
|---|---|---|---|---|
| | | N = 15 | N = 13 | N = 28 |
| Forced Expiratory Volume in | Mean (SD) | 3.41 (0.616) | 3.29 (0.461) | 3.35 (0.543) |
| 1 Second (L) | Median | 3.32 | 3.15 | 3.26 |
| | Range | 2.2-4.4 | 2.6-4.4 | 2.2-4.4 |
| Percent Predicted FEV1 (%) | Mean (SD) | 91.00 (10.623) | 92.08 (15.476) | 91.50 (12.855) |
| | Median | 93.00 | 87.00 | 90.00 |
| | Range | 71.0-108.0 | 67.0-118.0 | 67.0-118.0 |
| Methacholine PC20 (mg/mL) | Mean (SD) | 2.89 (2.967) | 2.29 (2.722) | 2.61 (2.820) |
| | Median | 2.34 | 1.24 | 1.37 |
| | Range | 0.1-10.2 | 0.3-9.6 | 0.1-10.2 |
| Fractional Exhaled Nitric | Mean (SD) | 61.83 (70.299) | 40.15 (32.042) | 51.77 (56.036) |
| Oxide (ppb) | | | | |
| | Median | 37.50 | 26.50 | 32.00 |
| | Range | 17.0-295.5 | 15.5-119.0 | 15.5-295.5 |
| Blood eosinophils (10E9/L) | Mean (SD) | 0.32 (0.264) | 0.20 (0.132) | 0.26 (0.217) |
| | Median | 0.18 | 0.13 | 0.15 |
| | Range | 0.1-1.0 | 0.1-0.4 | 0.1-1.0 |
| Sputum eosinophils (%) | Mean (SD) | 4.25 (5.873) | 4.42 (5.448) | 4.33 (5.565) |
| | Median | 1.70 | 1.55 | 1.60 |
| | Range | 0.7-21.3 | 0.0-14.8 | 0.0-21.3 |

The safety, PK, PD and full analysis sets were analyzed.

### Late Asthmatic Response (LAR):

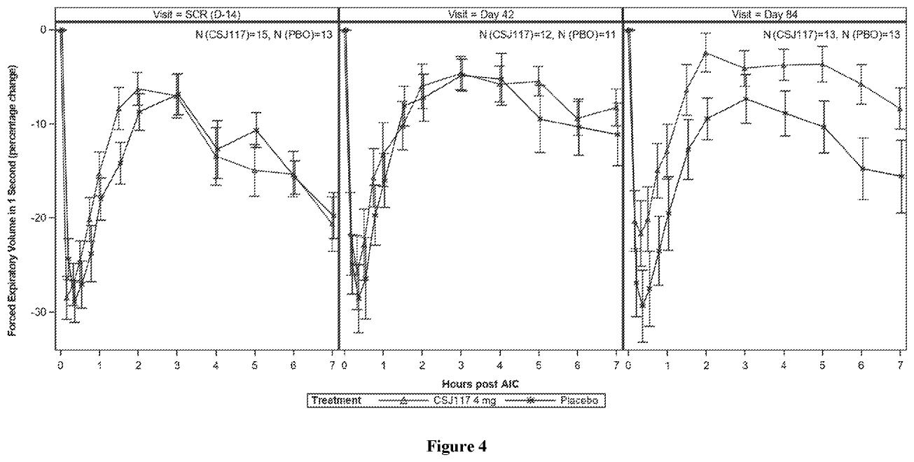

FIG. 4 shows the arithmetic mean of the percent change in FEV1 over the 7-hour period following the allergen inhalation challenges at screening and on Day 42 and Day 84. Dog, cat, horse, mouse, cattle, rat and bird allergens were included in the challenge. Table 6 and Table 7 show the fall in FEV1 values and percentages, respectively, after the allergen challenges.

The late asthmatic response (LAR) after an allergen inhalation challenge on Day 84 was evaluated by measuring the (1) area under the curve (AUC) time-adjusted percent fall in the FEV1 and (2) maximum percent fall in the FEV1 between 3 and 7 hours after study drug administration. On Day 84, the time-adjusted fall in FEV1 was statistically less between 3 and 7 hours after CSJ117 administration when compared with placebo (p-value of 0.008; Table 11-2). Table 6 shows the time-adjusted AUC percent fall values on Day 42 and 84. Similarly, the maximum fall in FEV1 was statistically different between 3 and 7 hours after CSJ117 administration on Day 84 (p-value of 0.029; Table 7). The minimum absolute FEV1 between 3 and 7 hours was statistically less after CSJ117 administration when compared with placebo on Day 84 (p-value of 0.050).

TABLE 6
ANCOVA of time-adjusted AUC % fall from pre-AIC FEV1-LAR
Pharmacodynamic analysis set.
Parameter (unit): LAR time adjusted AUC 3-7 h (%)

| | | | | | | Comparison of model-based | | |
|---|---|---|---|---|---|---|---|---|
| | | | | | | mean estimates | | |
| | | | | | | Test vs Ref. | | |
| | | | | | | Difference | | |
| | Test vs Ref | Model-based mean estimate (90% CI)* | | | | (CSJ117- | | p- |
| Visit | (Comparison) | Test | | Ref | | Placebo) | 90% CI* | value** |
| Day 42 | 4 mg (N = 12) vs | 13.11 | [8.64; 17.58] | 15.75 | [11.20; 20.29] | −2.64 | [−9.02; 3.74] | 0.243 |
| | Placebo (N = 11) | | | | | | | |
| Day 84 | 4 mg (N = 13) vs | 9.28 | [4.16; 14.39] | 17.70 | [12.59; 22.81] | −8.42 | [−15.66; −1.18] | 0.029 |
| | Placebo (N = 13) | | | | | | | |

*: two-sided Cls,
**: one-sided p-value
The endpoint was analyzed using an Analysis of Covariance (ANCOVA) model for repeated measures, to compare the CSJ117 group with placebo. The model included treatment and visit as independent variables, treatment by visit interaction term and baseline by visit interaction term. Baseline values were measured following the AIC at Day -14. An unstructured residual covariance structure was used. Estimates of the means and the mean differences were obtained through the ESTIMATE statement Hochberg procedure is used to control multiplicity of endpoints and control Type I error at 10% for each treatment difference.

TABLE 7
ANCOVA of time-adjusted AUC % fall from pre-AIC FEV1-LAR
Pharmacodynamic analysis set.
Parameter (unit): LAR time adjusted AUC 3-7 h (%)

| | | | | | | Comparison of model-based | | |
|---|---|---|---|---|---|---|---|---|
| | | | | | | mean estimates | | |
| | | | | | | Test vs Ref. | | |
| | | | | | | Difference | | |
| | Test vs Ref | Model-based mean estimate (90% CI)* | | | | (CSJ117- | | p- |
| Visit | (Comparison) | Test | | Ref | | Placebo) | 90% CI* | value** |
| Day 42 | 4 mg (N = 12) vs | 6.46 | [3.29; 9.63] | 9.36 | [6.14; 12.58] | −2.90 | [−7.43; 1.62] | 0.141 |
| | Placebo (N = 11) | | | | | | | |
| Day 84 | 4 mg (N = 13) vs | 4.20 | [0.84; 7.56] | 11.38 | [8.04; 14.73] | −7.18 | [−11.92; −2.44] | 0.008 |
| | Placebo (N = 13) | | | | | | | |

*: two-sided CIs, **: one-sided p-value
The endpoint was analyzed using an Analysis of Covariance (ANCOVA) model for repeated measures, to compare the CSJ117 group with placebo. The model included treatment and visit as independent variables, treatment by visit interaction term and baseline by visit interaction term. Baseline values were measured following the AIC at Day -14. An unstructured residual covariance structure was used. Estimates of the means and the mean differences were obtained through the ESTIMATE statement Hochberg procedure is used to control multiplicity of endpoints and control Type I error at 10% for each treatment difference.

### Early Asthmatic Response (EAR):

The early asthmatic response (EAR) after an allergen inhalation challenge on Day 42 and Day 84 was assessed by measuring the (1) area under the curve (AUC) time-adjusted percent fall in the FEV1, (2) maximum percent fall in the FEV1, and (3) minimum absolute FEV1 between 0 and 2 hours after study drug administration.

On Day 84, the time-adjusted fall in FEV1 between 0 and 2 hours was statistically less after CSJ117 administration when compared with placebo (p-value of 0.097; Table 8). The differences between treatment groups in maximum fall in FEV1 between 0 and 2 hours after study drug administration on Day 42 and Day 84 were not statistically significant (p-values of 0.172 and 0.105, respectively; 9), although the difference on Day 84 approached statistical significance.

TABLE 8
ANCOVA of time-adjusted AUC % fall from pre-AIC FEV1-LAR
Pharmacodynamic analysis set.
Parameter (unit): LAR time adjusted AUC 3-7 h (%)

| | | | | | | Comparison of model-based | | |
|---|---|---|---|---|---|---|---|---|
| | | | | | | mean estimates | | |
| | | | | | | Test vs Ref. | | |
| | | | | | | Difference | | |
| | Test vs Ref | Model-based mean estimate (90% CI)* | | | | (CSJ117- | | p- |
| Visit | (Comparison) | Test | | Ref | | Placebo) | 90% CI* | value** |
| Day 42 | 4 mg (N = 12) vs | 15.11 | [11.61; 18.61] | 9.36 | [12.57; 19.70] | −1.02 | [−6.06; 4.01] | 0.141 |
| | Placebo (N = 11) | | | | | | | |
| Day 84 | 4 mg (N = 13) vs | 12.83 | [9.01; 16.64] | 11.38 | [13.25; 20.92] | −4.26 | [−9.72; 1.21] | 0.008 |
| | Placebo (N = 13) | | | | | | | |

*: two-sided CIs, **: one-sided p-value
The endpoint was analyzed using an Analysis of Covariance (ANCOVA) model for repeated measures, to compare the CSJ117 group with placebo. The model included treatment and visit as independent variables, treatment by visit interaction term and baseline by visit interaction term. Baseline values were measured following the AIC at Day -14. An unstructured residual covariance structure was used.Estimates of the means and the mean differences were obtained through the ESTIMATE statement

TABLE 9
ANCOVA of time-adjusted AUC % fall from pre-AIC FEV1-LAR
Pharmacodynamic analysis set.
Parameter (unit): LAR time adjusted AUC 3-7 h (%)

| | | | | | | Comparison of model-based | | |
|---|---|---|---|---|---|---|---|---|
| | | | | | | mean estimates | | |
| | | | | | | Test vs Ref. | | |
| | | | | | | Difference | | |
| | Test vs Ref | Model-based mean estimate (90% CI)* | | | | (CSJ117- | | p- |
| Visit | (Comparison) | Test | | Ref | | Placebo) | 90% CI* | value** |
| Day 42 | 4 mg (N = 12) vs | 27.85 | [21.97; 33.73] | 32.56 | [26.57; 38.54] | −4.70 | [−13.10; 3.69] | 0.172 |
| | Placebo (N = 11) | | | | | | | |
| Day 84 | 4 mg (N = 13) vs | 25.48 | [20.39; 30.58] | 30.90 | [25.80; 36.00] | −5.41 | [−12.62; 1.79] | 0.105 |
| | Placebo (N = 13) | | | | | | | |

*: two-sided CIs, **: one-sided p-value
The endpoint was analyzed using an Analysis of Covariance (ANCOVA) model for repeated measures, to compare the CSJ117 group with placebo. The model included treatment and visit as independent variables, treatment by visit interaction term and baseline by visit interaction term. Baseline values were measured following the AIC at Day -14. An unstructured residual covariance structure was used. Estimates of the means and the mean differences were obtained through the ESTIMATE statement

### FeNO:

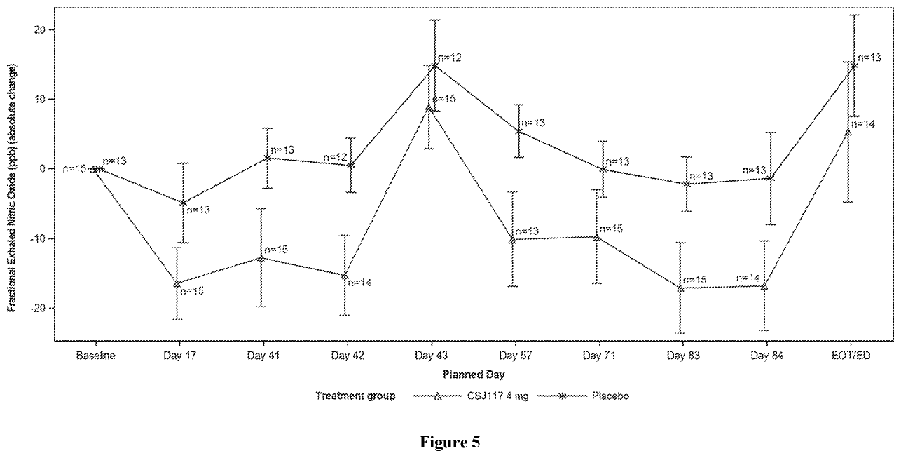

A trend for a reduction in fractional exhaled nitric oxide (FeNO) with CSJ117 as compared with placebo was observed over the 12-week treatment period (FIG. 5).

### Safety:

CSJ117 was safe and well tolerated in mild asthmatics. No adverse events (AE) imbalance observed across treatment groups. The rate of moderately severe adverse events were similar among subjects receiving CSJ117 (21.6%) and placebo (28.6%). No subject developed a severe or serious adverse event. No subject discontinued study drug due to an adverse event. The most frequent treatment-emergent adverse events were headache, nasopharyngitis, and oropharyngeal pain. No deaths or serious adverse events occurred. No clinically relevant changes were noted in the hematology, chemistry, urinalysis, ECG, and spirometry results.

### Example 3: Modelling Predictions of the CSJ117 Clinical Efficacious Dose Range

CSJ117 was tested in a randomized, double-blind, placebo-controlled proof-of-concept bronchoprovocation study with 28 mild, atopic asthma patients (Example 2). Patients were randomized to receive a 4 mg daily inhalation dose of CSJ117 or placebo for 12 weeks. The allergen challenge was conducted at screening, day 42 and day 84. The primary efficacy end point was the late asthmatic response (LAR) as measured 3 to 7 hours after the allergen challenge at day 84. The measures used to evaluate LAR were the area under the curve (AUC) of the time-adjusted decrease in the forced expiratory volume in 1 second (FEV1) and the maximum percentage decrease in FEV1. CSJ117 attenuated LAR at day 84 in the allergen challenge. During the late response, the AUC of the time-adjusted fall and the maximum fall % FEV1 were significantly less in the CSJ117 group, compared to placebo (4.20% vs 11.38%, p=0.008, and 9.28% vs 17.70%, P=0.029 respectively).

Simulation of Dose-Response Curves of CSJ117 from Doses Ranging from 0.5 to 8 mg:

% Efficacy of CSJ117 in PoC study compared to placebo was calculated using following formula: % E=[(B−A)/B]×100, where, E=Efficacy; A and B are LAR measured by max fall % FEV1 value in treated and placebo groups, respectively.

Published data from other widely asthma treatment budosenide/formoterol (ICS/LABA, inhaled corticosteroid+long-acting beta agonist) combination was used in a similar fashion to calculate its efficacy and results compared with CSJ117 in Table 10.

TABLE 10

| | | LAR | | | |
|---|---|---|---|---|---|
| | | Placebo | Treat- | | E, % |
| Treatment | Reference | (B) | ment (A) | B-A | [(B-A)/B]*100 |
| Budesonide/ | J ALLERGY | 21.2 | 4.2 | 17 | 80.2 |
| formoterol | CLIN | | | | |
| (400/12 μg) | IMMUNOL | | | | |
| maximal | VOLUME | | | | |
| fall % FEV1 | 119, | | | | |
| | NUMBER 2, | | | | |
| | pp 322-327 | | | | |
| CSJ117 | PoC study | 17.7 | 9.28 | 8.42 | 47.6 |
| (4 mg OD) | | | | | |
| Max fall % | | | | | |
| FEV1 | | | | | |

Maximum efficacy (Emax) of CSJ117 is unknown at this time and three potential values of Emax are possible:

1) 100% (if complete attenuation of LAR with CSJ117 is possible);
2) 80% (similar to reported efficacy of ICS/LABA); and
3) 60% (47.6% efficacy observed with 4 mg dose is 80% of Emax).

Hill equation was used to calculate ED50 for each value of Emax with Dose=4 mg, E=47.6%, and Hill slope, n=1 Hill Equation

$$\% E = \frac{E max \times {Dose}^{n}}{ED {50}^{n} + {Dose}^{n}}$$

Results are summarized in Table 11.

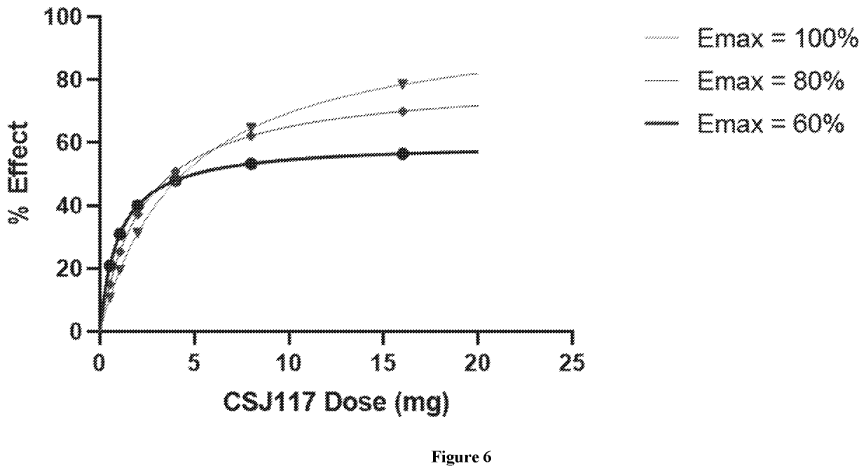

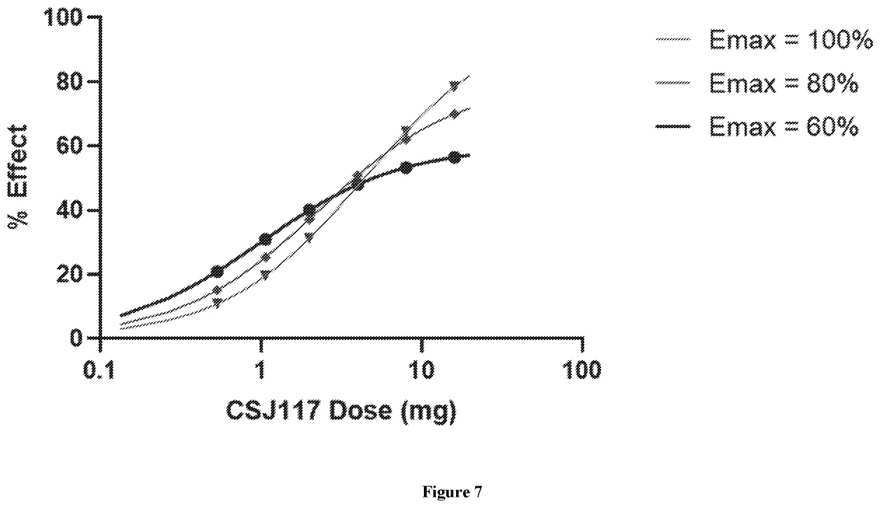

From Emax and ED50 in each scenario above, a dose-response curve was generated using Hill equation to assess range of efficacy values resulting from 0.5 to 8 mg once daily dose of CSJ117 (FIGS. 6 and 7). Efficacy due to 0.5 mg and 8 mg once daily dose of CSJ117 is predicted to range from 10-35% and 65-88% of maximum effect, respectively. As visible in plots of FIGS. 6 and 7, the planned dose range of 0.5, 1, 2, 4, and 8 mg o.d. of CSJ117 during Ph2B (Example 4) is expected to adequately describe the dose-response for CSJ117.

TABLE 11
Calculation (hill equation) of ED50 for Emax 100%, 80% and 60% with
Dose = 4 mg, E = 47.6%, and Hill slope, n = 1.

| | Emax, % | ED50, mg |
|---|---|---|
| Scenario 1 | 100 | 4.4 |
| Scenario 2 | 80 | 2.3 |
| Scenario 3 | 60 | 1.0 |

### Example 4: Phase 2b Clinical Trial: A 12-Week, Multicenter, Randomized, Double-Blind, Parallel-Arm, Placebo-Controlled Study to Assess the Efficacy and Safety of CSJ117, when Added to Existing Asthma Therapy in Patients ≥18 Years of Age with Severe Uncontrolled Asthma

The purpose of this study is to determine the efficacy and safety of multiple CSJ117 doses (0.5; 1; 2; 4 and 8 mg) inhaled once daily compared with placebo, when added to standard-of-care (SoC) asthma therapy in adult patients with uncontrolled asthma with respect to change from baseline in FEV1 at the end of 12 weeks of treatment.

### Objectives of the Study:

| Objective(s) | Endpoint(s) |
|---|---|
| Primary objective(s) | Endpoint(s) for primary objective(s) |
| To characterize the dose-response | Average change from baseline in pre- |
| relationship of five doses of CSJ117 | dose FEV1 (L) at Week 8 and Week 12. |
| inhaled daily on lung function, | |
| compared with placebo, at the end of the | |
| 12-week active-treatment period. | |
| Secondary objective(s) | Endpoint(s) for secondary objective(s) |
| To characterize the dose-response | Average change from baseline in FeNO |
| relationship of five doses of CSJ117 | at Week 8 and Week 12. |
| inhaled daily on FeNO compared with | Measurement of Anti-Drug Antibodies |
| placebo, during the 12-week active- | (ADA) titers at baseline and during the |
| treatment period | study (Week 0 [Day 1] to Week 24) |
| To assess immunogenicity of five doses | Measurement of total CSJ117 serum |
| of CSJ117 during the study | concentration during the study (Week 0 |
| To characterize the systemic | [Day 1] to Week 24) and calculation of |
| pharmacokinetic (PK) profile of multiple | PK parameters (e.g., Ctrough, Race, |
| inhaled daily doses of CSJ117 during | T %). |
| the 12 week, active treatment period and | Change from baseline in peak expiratory |
| the 12 week, follow-up period | flow (PEF; am and pm), as assessed by |
| To characterize the efficacy of five | mean morning and mean evening PEF in |
| doses of CSJ117 once daily, compared | each week (average over 7 days) during |
| with placebo, on peak expiratory flow | 12 weeks of treatment. |
| (PEF; AM and PM), as assessed by | Average change from baseline in ACQ-5 |
| mean morning and mean evening PEF | score at Week 8 and Week 12. |
| over the 12-week active-treatment | Average change from baseline in |
| period | AQLQ + 12 score at Week 8 and Week 12 |
| To characterize the efficacy of five | Average change from baseline |
| doses of CSJ117 once daily, compared | in ADSD and ANSD score at Week 8 |
| with placebo, on ACQ-5 over the 12- | and Week 12. |
| week active-treatment period. | Change from baseline in number of puffs |
| To characterize the efficacy of five | of SABA taken per day in each week |
| doses of CSJ117 once daily, compared | (average over 7 days) during 12 weeks |
| with placebo, on change from baseline | Summaries of treatment-emergent |
| in Asthma Quality of Life Questionnaire | adverse events, systolic and diastolic |
| (AQLQ) score at the end of the 12-week | blood pressure, pulse rate, body weight, |
| active-treatment period | ventricular rate, RR interval, PR interval, |
| To characterize the efficacy of five | QRS duration, heart rate, and |
| doses of CSJ117 once daily, compared | Fridericia’s QTc, laboratory values and |
| with placebo, on daytime and nighttime | change from baseline for continuous |
| asthma symptoms over the 12-week | laboratory values. |
| active-treatment period | |
| To characterize the efficacy of five | |
| doses of CSJ117 once daily, compared | |
| with placebo, on daily short-acting 3- | |
| agonist (SABA) use over the 12-week | |
| active-treatment period | |
| To assess the safety of five doses of | |
| CSJ117 once daily, compared with | |
| placebo, with respect to adverse events | |
| (AE), electrocardiograms (ECGs), vital | |
| signs and laboratory tests | |

### Study Design:

This study is a phase II, multicenter, multi-national, double-blind, randomized, parallel-arm placebo-controlled study to evaluate the effect of 5 dose levels of CSJ117 in adult subjects with inadequately controlled asthma despite medium to high dose ICS plus LABA. Approximately 625 patients are to be randomized into this study.

The study includes:
A screening epoch of up to 2 weeks to assess eligibility and assess baseline EOS count.
A single blinded placebo run-in epoch of 4 weeks, to collect baseline data for efficacy variables and compliance with the ePEF and eDiary device as well as to assess adherence with placebo and standardized maintenance medication, fluticasone propionate/salmeterol xinafoate 250/50 μg or 500/50 μg b.i.d. Eligibility for randomization is determined during the placebo run-in period.
A double blinded treatment epoch of 12 weeks.
A follow-up epoch of 12 weeks, study drug free, following the last dose of study drug.

At randomization, subjects are to be stratified by their blood eosinophil count (EOS) (≥300 or <300 cells/μl), measured at the screening visit. 80% total study population are to be enrolled in the high EOS stratum. Subjects are to be randomized in a 2:1:1:1:2:2 ratio to receive placebo or one of 5 doses of CSJ117 (0.5, 1, 2, 4, 8 mg) daily for 12 weeks. All arms have the same stratification ratio.

### Population:

The study population includes males and females aged >18 and <75 years, who are asthma patients already receiving ICS-LABA combination with up to two asthma controller medications.

### Key Inclusion Criteria:

Documented physician-diagnosed asthma (according to GINA 2019) for at least 12 months prior to screening.
Patients who have been treated with medium or high dose ICS plus LABA alone or with up to two additional to ICS-LABA controllers (allowed only: LTRA, theophylline or its derivatives or LAMA) in a stable dose must be stable for at least 1 month prior to Screening OR patients who have been treated o.d. with a fixed dose combination of 92 to 100 ug of fluticasone furoate and 22 to 25 ug of vilanterol alone or with up to two additional controllers (allowed only: LTRA, theophylline or its derivatives or LAMA) in a stable dose for at least 1 month prior to Screening.
Subjects must have a morning pre-BD FEV1 value of >40% and <85% of the predicted normal after withholding bronchodilators at the start and the end of Run-in.
Subjects must have a Post-BD reversibility of FEV1>12% and >200 mL within 30 minutes after administration of 400 μg salbutamol/albuterol (or equivalent dose) at the Run-in Visit.
Subjects must have an ACQ-5 score of >1.5 at Screening and End of Run-in visits.
Subjects must meet all of the following criteria at End of Run-In Visit prior to randomization:
Subjects must demonstrate acceptable inhaler, peak flow meter, and spirometry techniques during the run-in period (from beginning to end of run in).
Subjects must demonstrate ≥70% compliance with the asthma controller ICS-LABA during the last two weeks of the run-in period based on their inhaler use count. 70% compliance is defined as medication taken in 70% of the days in the last 2 weeks.
Subjects must demonstrate ≥70% compliance with the CSJ117 placebo/Concept1 during the last 2 weeks of the run-in period based on capsule use count. 70% compliance is defined as medication taken for 70% of the days in the last 2 weeks
Subjects must demonstrate ≥70% compliance with required use of the eDiary during the last 2 weeks of the run-in period. 70% compliance is defined as completing the daily eDiary for 70% of the days in the last 2 weeks

### Key Exclusion Criteria:

Patients with >2 asthma exacerbations requiring systemic corticosteroids, hospitalization or emergency room visit within 12 months prior to Screening
Patients, who cannot sustain washout of additional to ICS-LABA controllers before beginning of Run-In.
Any oral corticosteroids as a maintenance treatment within 3 months, or any use of short-acting, intra-articular, intramuscular, or intravenous corticosteroid within 1 month, or any use of long acting, intra-articular or intramuscular corticosteroids within 3 months prior to Screening
Patients who have smoked or inhaled any substance other than asthma medications (e.g. e-cigarettes, vapes, marijuana, etc.) within 6 months prior to Screening, or who have a cigarette smoking history of greater than 10 pack years.
Pregnant or nursing (lactating) women
Women of child-bearing potential, defined as all women physiologically capable of becoming pregnant, unless they are using specified methods of contraception during dosing of study drug and one week after last study drug treatment
Patients with a history of immunodeficiency disease or hepatitis B or hepatitis C.

### Rationale for Dose Regimen and Duration of Treatment

CSJ117 was designed for inhaled delivery. CSJ117 is formulated as a PulmoSol™ engineered powder in hard capsules for delivery to the lungs via dry powder inhaler (DPI; Concept1). Total duration of treatment with CSJ117 is 12 weeks. Total duration of treatment with placebo for all patients is either 4 weeks or 16 weeks, depending on randomization.

This dose range finding study includes 5 active doses of CSJ117 (0.5; 1.0; 2.0; 4.0 and 8.0 mg) and matching placebo for once daily inhaled delivery via Concept1 device. Based on modelling predictions of the CSJ117 clinical efficacious dose range and PK profile after repeated administration, once daily dosing over 12 weeks is expected to provide adequate pulmonary exposure.

From simulated plots of D-R curve, the planned dose range of 0.5, 1, 2, 4, and 8 mg o.d. of CSJ117 during Ph2B is expected to adequately describe the dose-response for CSJ117 (see Example 3). The 4 mg o.d. dose is selected because of its demonstrated efficacy after 12 weeks of administration in a bronchoprovocation study (Example 2) in mild atopic asthmatics. The 4 mg inhaled o.d. dose of CSJ117 is predicted to be efficacious in severe uncontrolled asthmatics. The lowest dose, 0.5 mg o.d., is intended to capture the slope of the dose-response curve (e.g., around the predicted ED50; the dose at which half of the maximum effect is reached). The top dose of 8 mg dose o.d. is selected to determine whether a plateau for efficacy has been reached. The 1 and 2 mg doses are included to adequately describe the efficacy dose-response.

### Example 5: PulmoSol Formulation Comprising Anti-TSLP Fab1 CSJ117

CSJ117 (anti-TSLP Fab1; Fab fragment) is formulated using PulmoSol technology, which involves solid-state stabilization of CSJ117 using an amorphous glassy matrix. Spray drying using an aqueous single-phase feedstock comprising of CSJ117 and excipients is utilized to produce respirable powders. The excipients and composition are selected to create a powder that comprised of core/shell morphology, having a core of CSJ117 stabilized within a glassy matrix surrounded by a shell of hydrophobic excipient that improves powder dispersibility.

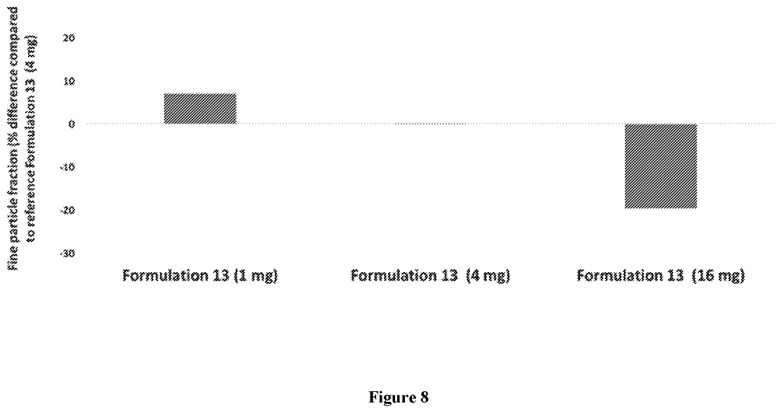

As described in WO2017/042701, one pharmaceutical intermediate strength (500 mg/g) with different target fill amounts was developed initially to achieve a wide dose range. However, this approach was giving different fine particle fraction (FPF, FIG. 8) per dosage strength and therefore variable dose delivery. In order to achieve wide dose range with constant FPF across dosage strengths, there is a need for therapeutic formulations with variable dosage strengths.

To improve the formulations previously described in WO2017/042701, the formulations 1 to 12 presented in Table 12 have been developed. Table 12 provides an overview of the FMI formulation. Trehalose is used a stabilizer and is required to create an amorphous glass which helps to dramatically reduce molecular mobility and motions in the anti-TSLP Fab1 molecule thus preventing physicochemical changes (e.g., aggregation and chemical modification). Trileucine is used as a shell former to design non cohesive spray-dried particles. Trileucine improves the powder aerosolization behavior and dispersibility, providing higher delivered dose, which potentially allows for a reduction of the drug loading in the formulation while maintaining the amount delivered to the lungs.

TABLE 12
Anti-TSLP Fab1 (CSJ117) Formulations.

| | | | | | | | Drug |
|---|---|---|---|---|---|---|---|
| | ANTI- | | | | | | product |
| For- | TSLP | | | | | Sugar: | dose |
| mulation | Fab1 | Trehalose | Trileucine | Histidine | HCl | Protein ratio | strength |
| number | (% w/w) | (% w/w) | (% w/w) | (% w/w) | (% w/w) | (w:w) | (mg) |
| 1 | 3.13 | 80.83 | 10.00 | 5.01 | 1.04 | 25.82 | 0.5 |
| 2 | 5.00 | 70.91 | 15.00 | 7.53 | 1.56 | 14.18 | 0.5 |
| 3 | 6.25 | 77.61 | 10.00 | 5.09 | 1.05 | 12.42 | 1 |
| 4 | 10.00 | 65.76 | 15.00 | 7.66 | 1.59 | 6.58 | 1 |
| 5 | 10.00 | 73.73 | 10.00 | 5.19 | 1.08 | 7.37 | 1 |
| 6 | 12.50 | 71.15 | 10.00 | 5.25 | 1.09 | 5.69 | 2 |
| 7 | 20.00 | 55.44 | 15.00 | 7.92 | 1.64 | 2.77 | 2 |
| 8 | 25.00 | 50.29 | 15.00 | 8.05 | 1.67 | 2.01 | 4 |
| 9 | 25.00 | 58.26 | 10.00 | 5.58 | 1.16 | 2.33 | 4 |
| 10 | 40.00 | 34.81 | 15.00 | 8.43 | 1.75 | 0.87 | 4 |
| 11 | 50.00 | 24.50 | 15.00 | 8.69 | 1.81 | 0.49 | 8, 16 |
| 12 | 50.00 | 32.48 | 10.00 | 6.22 | 1.30 | 0.65 | 8, 16 |
| 13 | 50.00 | 19.22 | 15.00 | 13.04 | 2.74 | 0.38 | 1, 4, 16 |

Formulations 1 to 12, presented in Table 12 demonstrated to have high stability of anti-TSLP Fab1 in the dried state and high delivery efficiency. Furthermore, the wide range of drug loading tested, allows to cover extreme dosage strengths, e.g. from 0.5 to 16 mg per capsule, without relevant impact on fine particle fraction. The mostly increased and fixed target fill weights per capsule, make formulations 1 to 12 particularly suitable for commercial applications compared to formulation 13.

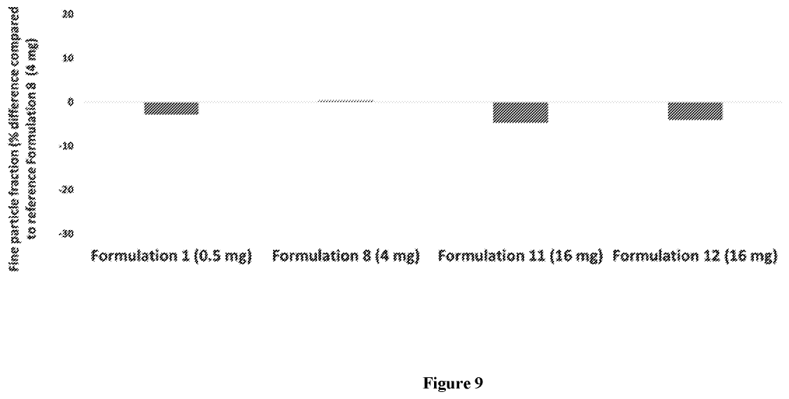

It was demonstrated that the formulations of Table 12 have high (>50%) and consistent fine particle fraction and dose linear fine particle mass across DP dosage strengths from 0.5 mg to 16 mg of anti-TSLP Fab1 per capsule (FIG. 9). FPF was not impacted by variation of tri-leucine content. This was improved in comparison to formulation 13 (FIG. 8).

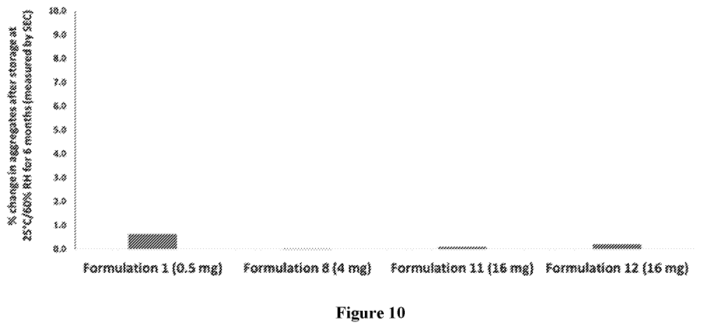

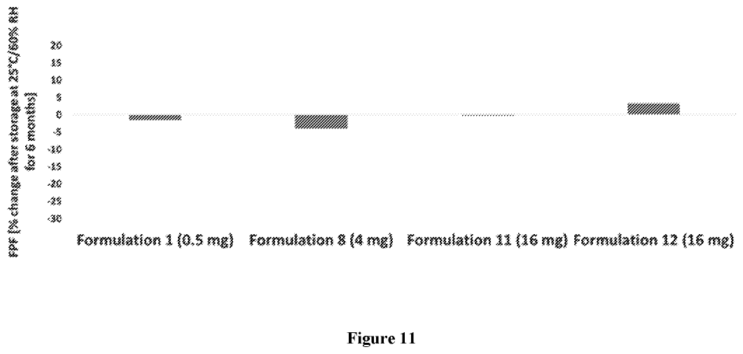

All anti-TSLP Fab1 Pulmosol formulations of Table 12 had negligible increase in aggregation as measured by size exclusion chromatography (SEC). They were stable for at least 6 months at 25° C./60° C. (FIG. 10). This was independent from the dosage strength and the tri-leucine levels applied. Furthermore, fine particle fraction measured by NGI was consistent over stability across all dosage strengths and tri-leucine levels (FIG. 11).

Anti-TSLP Fab1 is stabilized in dried state achieving room temperature stability, achieve a feed concentration of 1-5% w/w which increases the process throughput (for scale up). In addition, the current formulation enables low dose (DP ˜0.5 mg) delivery to the lung at 16.5 mg fill mass in the capsule.

### Example 6: Phase 2 Clinical Trial: Study of Effect of CSJ117 on Symptoms, Pharmacodynamics and Safety in Patients with Chronic Obstructive Pulmonary Disease (COPD)

Chronic obstructive pulmonary disease (COPD) is characterized by persistent respiratory symptoms (dyspnea, cough, sputum production) and airflow limitation (diagnosed using spirometry) that is due to airway and/or alveolar abnormalities usually caused by significant exposure to noxious particles/gases, in particular cigarette smoke. COPD is highly prevalent and represents an important public health challenge in all countries. It affects over million people in the United States of America (US) alone (National Center for Health et al 2017), and is the third leading cause of death worldwide (World Health Organization 2018). While currently available drug treatments for COPD (primarily beta-agonists, anticholinergics and corticosteroids) improve dyspnea, increase quality of life and decrease the number of exacerbations, the majority of patients remain symptomatic and many continue to suffer from exacerbations. There is a high unmet need for new therapeutic options with few products currently in development for COPD.

The purpose of this study is to assess in patients with COPD the efficacy of 2 dose levels of CSJ117 in comparison to placebo on disease symptom burden, lung function, lung structure, and biomarkers indicative of disease and target pathway modulation as well as PK and safety/tolerability.

### Objectives of the Study:

| Objective(s) | Endpoint(s) |
|---|---|
| Primary objective(s) | Endpoint(s) for primary objective(s) |
| To assess the effect of | Change in E-RS (Evaluating Respiratory |
| CSJ117 on disease/symptom | Symptoms) score from baseline |
| burden | |
| Secondary objective(s) | Endpoint(s) for secondary objective(s) |
| To assess the effect of | Change from baseline in COPD Assessment Test |
| CSJ117 on disease/symptom | (CAT), St. George’s Respiratory Questionnaire |
| burden | (SGRQ) |
| | Change from baseline in domains/items of |
| | respective PROs including E-RS |
| To assess the number and | Response in E-RS, total score ≥ 1.5 points decrease |
| percentage of patients who | from baseline |
| respond to treatment with | Response in CAT, total score ≥ 22 points decrease |
| CSJ117 with respect to | from baseline |
| improvement of symptoms | Response in SGRQ, total score ≥ 24 points decrease |
| | from baseline |
| To assess the effect of | Change from baseline in lung function as assessed |
| CSJ117 on lung function | in spirometry |
| | % of patients achieving ≥ 100 mL of increase in |
| | change from baseline in pre- and post |
| | bronchodilator FEV1 |
| To assess the effect of | Puffs of rescue med per day as captured by |
| CSJ117 on use of rescue | electronic diary |
| medication (SABA, SAMA) | |
| To assess the effect of | Rate of, time to, and severity of exacerbation (using |
| CSJ117 on COPD | the EXACT instrument) |
| exacerbations | |
| To assess the safety of two | Summaries of treatment-emergent adverse events, |
| doses of CSJ117 once daily, | systolic and diastolic blood pressure, pulse rate, |
| compared with placebo, with | body weight, ventricular rate, RR interval, PR |
| respect to adverse events | interval, QRS duration, heart rate, and Fridericia’s |
| (AE), electrocardiograms | QTc, laboratory values and change from baseline |
| (ECGs), vital signs and | for continuous laboratory values. |
| laboratory tests | |

### Study Design:

This is a randomized, participant- and investigator-blinded, placebo-controlled, parallel group, multicenter study with two fixed-dose levels of CSJ117 (4 mg, 8 mg) administered once-daily via oral inhalation over 12 weeks in patients with COPD.

Participants undergo screening assessments prior to entering the 2 week run-in period. During the run-in period, participants are on triple background therapy (ICS/LABA/LAMA, e.g. Trelegy or Trimbow) that is carried forward during the entire study. Following the run-in period, participants undergo baseline assessments prior to entering the treatment period.

Study treatment: CSJ117 8 mg inhaled once daily, CSJ117 4 mg inhaled once daily, matching placebo for 12 weeks:
Treatment arm 1: CSJ117, 8 mg, q.d.
Treatment arm 2: CSJ117, 4 mg, q.d.
Treatment arm 3: Placebo matching CSJ117 q.d
COPD maintenance background therapy ICS/LABA/LAMA, e.g., a combination of fluticasone furoate, vilanterol, and umeclidinium (Trelegy®), or a combination of beclometasone, formoterol, and glycopyrronium (Trimbow®)

CSJ117 and matching placebo is provided as powder filled capsules with a Concept1 inhalation device.

### Population:

The study population includes male and female patients (>40 years of age) with moderate to severe COPD (GOLD 2 and 3). Patients are prone for exacerbations (>2 moderate/severe exacerbations or >1 hospitalization for exacerbation in past 18 months), symptomatic at baseline (CAT>15) despite triple (ICS+LABA+LAMA) therapy.

### Key Inclusion Criteria

Participants eligible for inclusion in this study must meet all of the following criteria:
Able to provide written informed consent
Male and female adult patients aged >40 years at screening visit
Able to perform acceptable and reproducible spirometry with post-bronchodilator FEV1/FVC<0.7 and post-bronchodilator FEV1>30 and <80% of predicted at the screening and baseline visits (GOLD stage II or III COPD)
CAT>=15 points prior to randomization
Patients with a physician-diagnosed history of COPD for at least 1 year prior to screening visit
Documented history of at least two moderate to severe COPD exacerbation or 1 hospitalization for a COPD exacerbation in past 18 months
Current or ex-smokers who have a smoking history of at least 10 pack-years

### Kev Exclusion Criteria

Participants meeting any of the following criteria are not eligible for inclusion in this study:
Patients with a past or current medical history of asthma
Patients with a current or past medical history of conditions other than COPD or allergic rhinitis that could result in elevated circulating eosinophils levels (e.g., asthma, hypereosinophilic syndrome, Churg-Strauss Syndrome). Patients with known parasitic infestation within 6 months prior to screening are also excluded.
Patients with a clinical diagnosis of α-1 anti-trypsin deficiency (heterozygous genotype may be enrolled)
Patients with history of concomitant chronic or severe pulmonary disease (e.g., sarcoidosis, interstitial lung disease, cystic fibrosis, tuberculosis). Exception: Patients with concomitant mild or moderate pulmonary hypertension or bronchiectasis are permitted to participate
Patients on prohibited treatment listed in the Treatment section below
Use of other investigational drugs at the time of enrollment, or within 5 half-lives of experimental drug at the time of enrollment, or within 30 days of last dose of experimental drug at the time of enrollment, whichever is longer; or longer if required by local regulations, as listed in the clinical study protocol

1. A method of treating an inflammatory or obstructive airway disease comprising administering to a subject in need thereof a dose of about 2 mg to about 16 mg of an anti-TSLP antibody or anti-TSLP antibody fragment.

2. A method of treating asthma or reducing the frequency and/or severity of asthma exacerbations in a subject or reducing ACQ-5 score in a subject or treating chronic obstructive pulmonary disease (COPD) or reducing the frequency and/or severity of COPD exacerbations in a subject or reducing EXACT—Respiratory Symptoms (E-RS) score in a subject or improving asthma or COPD symptoms in a subject, comprising administering to a subject in need thereof a therapeutically effective amount of an anti-TSLP antibody or anti-TSLP antibody fragment, and wherein
a) the subject has a non-eosinophilic profile or a low eosinophil profile,
b) the subject has a Th2 low profile, and/or
c) the therapeutically effective amount is a dose of about 2 mg to about 16 mg of anti-TSLP antibody or anti-TSLP antibody fragment.

3-4. (canceled)

5. The method of claim 1, wherein the antibody or antibody fragment is administered at a dose of:
a) about 2 mg; or
b) about 4 mg; or
c) about 8 mg; or
d) about 16 mg.

6. The method of claim 1, wherein the antibody or antibody fragment is administered at said dose once a day.

7. The method of claim 1, wherein the antibody or antibody fragment is administered for a period of at least 1 month, at least 2 months, at least 3 months, at least 4 months, at least 6 months, at least 9 months, or at least 1 year or more.

8. The method of claim 1, wherein the antibody or antibody fragment is administered to the subject orally or intranasally.

9. The method of claim 8, wherein the antibody or antibody fragment is administered to the subject by inhalation.

10. The method of claim 1, wherein the subject has allergic asthma or non-allergic asthma.

11. The method of claim 1, wherein the subject has mild asthma.

12. (canceled)

13. The method of claim 1, wherein the subject has moderate, moderate-to-severe asthma or severe asthma.

14. The method of claim 13, wherein the subject has uncontrolled asthma.

15. The method of claim 1, wherein the subject has eosinophilic or non-eosinophilic asthma.

16. The method of claim 1, wherein the subject has eosinophilic or non-eosinophilic COPD.

17. The method of claim 1, wherein the subject has a high eosinophil count.

18. The method of claim 1, wherein the subject has a low eosinophil count.

19. The method of claim 1, wherein the subject has a Th2 low profile.

20. The method of claim 19, wherein the subject has a Th2 profile of IgE less than or equal to 100 IU/ml or an eosinophil count of less than 140 cells/μl.

21. The method of claim 1, wherein the subject is on background therapy of:
(a) medium or high dose corticosteroids; or
(b) medium or high dose corticosteroids in combination with LABA (ICS/LABA); or
(c) medium or high dose corticosteroids in combination with LABA and up to two additional ICS-LABA controllers.

22. The method of claim 1, wherein the subject is selected according to at least one of the following criteria:
a) prior to treatment with the antibody or antibody fragment, the subject has a post-bronchodilator reversibility of FEV1≥12% and ≥200 mL;
b) prior to treatment with the antibody or antibody fragment, the subject has a spirometry with pre-bronchodilator FEV1≥40% and ≤85%; and/or
c) prior to treatment with the antibody or antibody fragment, the subject has an ACQ-5 score ≥1.5.

23. The method of claim 1, wherein said subject achieves by week 12 of the treatment at least one of the following:
a) an improvement in FVC;
b) an improvement in the FEV1;
c) an improvement of FeNO;
d) an improvement in morning and evening peak expiratory flow rate (PEF);
e) an improvement of one or more symptoms of asthma as measured by an asthma symptom diary;
f) a reduction in the ACQ-5 score; and/or
g) a reduction of Asthma Quality of Life Questionnaire (AQLQ) score.

24. The method of claim 1, wherein the administration decreases eosinophils in blood, sputum, broncheoalveolar fluid, or lungs of the subject.

25. The method of claim 1, wherein the administration decreases Th2 cell count or shifts cell counts in the subject from a Th2 high population to a Th2 low population.

26. The method of claim 1, wherein the administration delays the time to an asthma or COPD exacerbation compared to a subject not receiving the anti-TSLP antibody or anti-TSLP antibody fragment.

27. The method of claim 1, wherein the anti-TSLP antibody or anti-TSLP antibody fragment is selected from any one of the following:
a) an antibody or antibody fragment that comprises:
a HCDR1 comprising the amino acid sequence of SEQ ID NO: 4;
a HCDR2 comprising the amino acid sequence of SEQ ID NO: 2;
a HCDR3 comprising the amino acid sequence of SEQ ID NO: 3;
a LCDR1 comprising the amino acid sequence of SEQ ID NO: 11;
a LCDR2 comprising the amino acid sequence of SEQ ID NO: 12; and
a LCDR3) comprising the amino acid sequence of SEQ ID NO: 13;
b) an antibody or antibody fragment that comprises:
a HCDR1 comprising the amino acid sequence of SEQ ID NO: 5;
a HCDR2 comprising the amino acid sequence of SEQ ID NO: 6;
a HCDR3 comprising the amino acid sequence of SEQ ID NO: 3;
a LCDR1 comprising the amino acid sequence of SEQ ID NO: 14;
a LCDR2 comprising the amino acid sequence of SEQ ID NO: 15; and
a LCDR3 comprising the amino acid sequence of SEQ ID NO: 16;
c) an antibody or antibody fragment that comprises:
a HCDR1 comprising the amino acid sequence of SEQ ID NO: 1;
a HCDR2 comprising the amino acid sequence of SEQ ID NO: 2;
a HCDR3 comprising the amino acid sequence of SEQ ID NO: 3;
a LCDR1 comprising the amino acid sequence of SEQ ID NO: 11;
a LCDR2 comprising the amino acid sequence of SEQ ID NO: 12; and
a LCDR3 comprising the amino acid sequence of SEQ ID NO: 13;
d) an antibody or antibody fragment that comprises:
a HCDR1 comprising the amino acid sequence of SEQ ID NO: 28;
a HCDR2 comprising the amino acid sequence of SEQ ID NO: 29;
a HCDR3 comprising the amino acid sequence of SEQ ID NO: 30;
a LCDR1 comprising the amino acid sequence of SEQ ID NO: 25;
a LCDR2 comprising the amino acid sequence of SEQ ID NO: 26; and
a LCDR3 comprising the amino acid sequence of SEQ ID NO: 27;
e) an antibody or antibody fragment that comprises:
a heavy chain variable region comprising
i. the amino acid sequence of SEQ ID NO: 7, or
ii. a sequence at least 90% identical to SEQ ID NO: 7, or
iii. a conservative variant of SEQ ID NO: 7; and
a light chain variable region comprising
i. the amino acid sequence of SEQ ID NO: 17, or
ii. a sequence at least 90% identical to SEQ ID NO: 17, or
iii. a conservative variant of SEQ ID NO: 17:
f) an antibody or antibody fragment that comprises:
a heavy chain variable region comprising
i. the amino acid sequence of SEQ ID NO: 32, or
ii. a sequence at least 90% identical to SEQ ID NO: 32, or
iii. a conservative variant of SEQ ID NO: 32; and
a light chain variable region comprising
i. the amino acid sequence of SEQ ID NO: 31, or
ii. a sequence at least 90% identical to SEQ ID NO: 31, or
iii. a conservative variant of SEQ ID NO: 31;
g) an antibody or antibody fragment that comprises:
a heavy chain comprising
i. the amino acid sequence of SEQ ID NO: 9, or
ii. a sequence at least 90% identical to SEQ ID NO: 9, or
iii. a conservative variant of SEQ ID NO: 9, and
a light chain comprising
i. the amino acid sequence of SEQ ID NO: 19, or
ii. a sequence at least 90% identical to SEQ ID NO: 19, or
iii. a conservative variant of SEQ ID NO: 19; and
h) an antibody or antibody fragment that comprises:
a heavy chain
i. the amino acid sequence of SEQ ID NO: 33, or
ii. a sequence at least 90% identical to SEQ ID NO: 33, or
iii. a conservative variant of SEQ ID NO: 33, and
a light chain comprising
i. the amino acid sequence of SEQ ID NO: 34, or
ii. a sequence at least 90% identical to SEQ ID NO: 34, or
iii. a conservative variant of SEQ ID NO: 34.

28-32. (canceled)

33. The method of claim 1, wherein the anti-TSLP antibody or anti-TSLP antibody fragment is administered at a dose of 70 mg to 280 mg at an interval of every 2 weeks or every 4 weeks.

34. The method of claim 1, wherein the antibody fragment is selected from the group consisting of a Fab, Fab′, F(ab′)2, scFv, minibody, or diabody.

35. The method of claim 1, wherein the antibody is a human immunoglobulin.

36. The method of claim 1, further comprising administering a second agent to the subject.

37. The method of claim 36, wherein the second agent is selected from the group consisting of
a) corticosteroids;
b) bronchodilators;
c) leukotriene receptor antagonists (LTRA),
d) long-acting anti-muscarinics (LAMA);
e) cromones;
f) antihistamines;
g) antileukotrienes; and
h) PDE-4 inhibitors.

38. The method of claim 1, wherein the administration of the antibody or antibody fragment eliminates or reduces the need for second agent therapy.

39-48. (canceled)

49. A pharmaceutical composition comprising spray-dried particles comprising:
i. a core comprising an anti-TSLP antibody or anti-TSLP antibody fragment,
wherein said anti-TSLP antibody or anti-TSLP antibody fragment is about 1% to about 70% (w/w) of the composition; and
ii. a shell comprising trileucine, wherein trileucine is about 1% to about 25% (w/w).

50. The pharmaceutical composition of claim 49, wherein the composition comprises 0.5 mg to 16 mg of the anti-TSLP antibody or anti-TSLP antibody fragment.

51. The pharmaceutical composition of claim 49, wherein the core further comprises trehalose.

52. The pharmaceutical composition of claim 49, wherein the composition has a pH of 4.5 to 6.0.

53. The pharmaceutical composition of claim 49, wherein the composition further comprises a buffer.

54. The pharmaceutical composition of claim 49, wherein the anti-TSLP antibody or anti-TSLP antibody fragment is selected from any one of the following:
a) an antibody or antibody fragment that comprises:
a HCDR1 comprising the amino acid sequence of SEQ ID NO: 4;
a HCDR2 comprising the amino acid sequence of SEQ ID NO: 2;
a HCDR3 comprising the amino acid sequence of SEQ ID NO: 3;
a LCDR1 comprising the amino acid sequence of SEQ ID NO: 11;
a LCDR2 comprising the amino acid sequence of SEQ ID NO: 12; and
a LCDR3 comprising the amino acid sequence of SEQ ID NO: 13;
b) an antibody or antibody fragment that comprises:
a HCDR1 comprising the amino acid sequence of SEQ ID NO: 5;
a HCDR2 comprising the amino acid sequence of SEQ ID NO: 6;
a HCDR3 comprising the amino acid sequence of SEQ ID NO: 3;
a LCDR1 comprising the amino acid sequence of SEQ ID NO: 14;
a LCDR2 comprising the amino acid sequence of SEQ ID NO: 15; and
a LCDR3 comprising the amino acid sequence of SEQ ID NO: 16;
c) an antibody or antibody fragment that comprises:
a HCDR1 comprising the amino acid sequence of SEQ ID NO: 1;
a HCDR2 comprising the amino acid sequence of SEQ ID NO: 2;
a HCDR3 comprising the amino acid sequence of SEQ ID NO: 3;
a LCDR1 comprising the amino acid sequence of SEQ ID NO: 11;
a LCDR2 comprising the amino acid sequence of SEQ ID NO: 12; and
a LCDR3 comprising the amino acid sequence of SEQ ID NO: 13;
d) an antibody or antibody fragment that comprises:
a HCDR1 comprising the amino acid sequence of SEQ ID NO: 28;
a HCDR2 comprising the amino acid sequence of SEQ ID NO: 29;
a HCDR3 comprising the amino acid sequence of SEQ ID NO: 30;
a LCDR1 comprising the amino acid sequence of SEQ ID NO: 25;
a LCDR2 comprising the amino acid sequence of SEQ ID NO: 26; and
a LCDR3 comprising the amino acid sequence of SEQ ID NO: 27;
e) an antibody or antibody fragment that comprises:
a heavy chain variable region comprising
i. the amino acid sequence of SEQ ID NO: 7, or
ii. a sequence at least 90% identical to SEQ ID NO: 7, or
iii. a conservative variant of SEQ ID NO: 7; and
a light chain variable region comprising
i. the amino acid sequence of SEQ ID NO: 17, or
ii. a sequence at least 90% identical to SEQ ID NO: 17, or
iii. a conservative variant of SEQ ID NO: 17:
f) an antibody or antibody fragment that comprises:
a heavy chain variable region comprising
i. the amino acid sequence of SEQ ID NO: 32, or
ii. a sequence at least 90% identical to SEQ ID NO: 32, or
iii. a conservative variant of SEQ ID NO: 32; and
a light chain variable region comprising
i. the amino acid sequence of SEQ ID NO: 31, or
ii. a sequence at least 90% identical to SEQ ID NO: 31, or
iii. a conservative variant of SEQ ID NO: 31:
g) an antibody or antibody fragment that comprises:
a heavy chain comprising
i. the amino acid sequence of SEQ ID NO: 9, or
ii. a sequence at least 90% identical to SEQ ID NO: 9, or
iii. a conservative variant of SEQ ID NO: 9, and
a light chain comprising
i. the amino acid sequence of SEQ ID NO: 19, or
ii. a sequence at least 90% identical to SEQ ID NO: 19, or
iii. a conservative variant of SEQ ID NO: 19; and
h) an antibody or antibody fragment that comprises:
a heavy chain
i. the amino acid sequence of SEQ ID NO: 33, or
ii. a sequence at least 90% identical to SEQ ID NO: 33, or
iii. a conservative variant of SEQ ID NO: 33, and
a light chain comprising
i. the amino acid sequence of SEQ ID NO: 34, or
ii. a sequence at least 90% identical to SEQ ID NO: 34, or
iii. a conservative variant of SEQ ID NO: 34.

55-56. (canceled)

57. The pharmaceutical composition of claim 49, wherein the antibody fragment is selected from the group consisting of a Fab, Fab′, F(ab′)2, scFv, minibody, or diabody.

58. The pharmaceutical composition of claim 49, wherein the composition comprises one or more of the following:
a) about 3-3.5% (w/w), about 6-6.5% (w/w), about 12-13% (w/w), about 25% (w/w), or about 50% (w/w) of the anti-TSLP antibody or anti-TSLP antibody fragment;
b) about 10% to about 95% (w/w) trehalose;
c) about 2% (w/w) to about 15% (w/w) histidine; and
d) about 0.5% (w/w) to about 4% (w/w) HCl.

59. (canceled)

60. The pharmaceutical composition of claim 51, wherein trehalose: anti-TSLP antibody or anti-TSLP antibody fragment ratio is greater than 0.4, greater than 0.5, greater than 0.6, greater than 0.65, greater than 1, greater than 1.5, greater than 2, greater than 3, greater than 4, greater than 5, greater than 10, greater than 15, greater than 20, or greater than 25.

61-62. (canceled)

63. The pharmaceutical composition of claim 49, wherein the composition comprises:
a) about 3% (w/w) anti-TSLP antibody or anti-TSLP antibody fragment, about 10% (w/w) trileucine, about 81% (w/w) trehalose, and a buffer;
b) about 5% (w/w) anti-TSLP antibody or anti-TSLP antibody fragment, about 15% (w/w) trileucine, about 71% (w/w) trehalose, and a buffer;
c) about 6% (w/w) anti-TSLP antibody or anti-TSLP antibody fragment, about 10% (w/w) trileucine, about 78% (w/w) trehalose, and a buffer;
d) about 10% (w/w) anti-TSLP antibody or anti-TSLP antibody fragment, about 15% (w/w) trileucine, about 66% (w/w) trehalose, and a buffer;
e) about 10% (w/w) anti-TSLP antibody or anti-TSLP antibody fragment, about 10% (w/w) trileucine, about 74% (w/w) trehalose, and a buffer;
f) about 12.5% (w/w) anti-TSLP antibody or anti-TSLP antibody fragment, about 10% (w/w) trileucine, about 71% (w/w) trehalose, and a buffer;
g) about 20% (w/w) anti-TSLP antibody or anti-TSLP antibody fragment, about 15% (w/w) trileucine, about 55.5% (w/w) trehalose, and a buffer;
h) about 25% (w/w) anti-TSLP antibody or anti-TSLP antibody fragment, about 15% (w/w) trileucine, about 50% (w/w) trehalose, and a buffer;
i) about 25% (w/w) anti-TSLP antibody or anti-TSLP antibody fragment, about 10% (w/w) trileucine, about 58% (w/w) trehalose, and a buffer;
j) about 40% (w/w) anti-TSLP antibody or anti-TSLP antibody fragment, about 15% (w/w) trileucine, about 35% (w/w) trehalose, and a buffer;
k) about 50% (w/w) anti-TSLP antibody or anti-TSLP antibody fragment, about 15% (w/w) trileucine, about 24.5% (w/w) trehalose, and a buffer; or
l) about 50% (w/w) anti-TSLP antibody or anti-TSLP antibody fragment, about 10% (w/w) trileucine, about 32.5% (w/w) trehalose, and a buffer.

64. The pharmaceutical composition of claim 63, wherein the composition comprises:
a) about 3% (w/w) anti-TSLP antibody or anti-TSLP antibody fragment, about 10% (w/w) trileucine, about 81% (w/w) trehalose, about 5% (w/w) histidine, and about 1% (w/w) HCl;
b) about 5% (w/w) anti-TSLP antibody or anti-TSLP antibody fragment, about 15% (w/w) trileucine, about 71% (w/w) trehalose, about 7.5% (w/w) histidine, and about 1.5% (w/w) HCl;
c) about 6% (w/w) anti-TSLP antibody or anti-TSLP antibody fragment, about 10% (w/w) trileucine, about 78% (w/w) trehalose, about 5% (w/w) histidine, and about 1% (w/w) HCl;
d) about 10% (w/w) anti-TSLP antibody or anti-TSLP antibody fragment, about 15% (w/w) trileucine, about 66% (w/w) trehalose, about 7.5% (w/w) histidine, and about 1.5% (w/w) HCl;
e) about 10% (w/w) anti-TSLP antibody or anti-TSLP antibody fragment, about 10% (w/w) trileucine, about 74% (w/w) trehalose, about 5% (w/w) histidine, and about 1% (w/w) HCl;
f) about 12.5% (w/w) anti-TSLP antibody or anti-TSLP antibody fragment, about 10% (w/w) trileucine, about 71% (w/w) trehalose, about 5% (w/w) histidine, and about 1% (w/w) HCl;
g) about 20% (w/w) anti-TSLP antibody or anti-TSLP antibody fragment, about 15% (w/w) trileucine, about 55.5% (w/w) trehalose, about 8% (w/w) histidine, and about 1.5% (w/w) HCl;
h) about 25% (w/w) anti-TSLP antibody or anti-TSLP antibody fragment, about 15% (w/w) trileucine, about 50% (w/w) trehalose, about 8% (w/w) histidine, and about 2% (w/w) HCl;
i) about 25% (w/w) anti-TSLP antibody or anti-TSLP antibody fragment, about 10% (w/w) trileucine, about 58% (w/w) trehalose, about 6% (w/w) histidine, and about 1% (w/w) HCl;
j) about 40% (w/w) anti-TSLP antibody or anti-TSLP antibody fragment, about 15% (w/w) trileucine, about 35% (w/w) trehalose, about 8% (w/w) histidine, and about 2% (w/w) HCl;
k) about 50% (w/w) anti-TSLP antibody or anti-TSLP antibody fragment, about 15% (w/w) trileucine, about 24.5% (w/w) trehalose, about 8.5% (w/w) histidine, and about 2% (w/w) HCl; or
l) about 50% (w/w) anti-TSLP antibody or anti-TSLP antibody fragment, about 10% (w/w) trileucine, about 32.5% (w/w) trehalose, about 6% (w/w) histidine, and about 1.5% (w/w) HCl.

65. The pharmaceutical composition of claim 49, wherein the composition comprises:
a) 3.13% (w/w) anti-TSLP antibody or anti-TSLP antibody fragment, 10% (w/w) trileucine, 80.83% (w/w) trehalose, 5.01% (w/w) histidine, and 1.04% (w/w) HCl;
b) 5% (w/w) anti-TSLP antibody or anti-TSLP antibody fragment, 15% (w/w) trileucine, 70.97% (w/w) trehalose, 7.53% (w/w) histidine, and 1.56% (w/w) HCl;
c) 6.25% (w/w) anti-TSLP antibody or anti-TSLP antibody fragment, 10% (w/w) trileucine, 77.61% (w/w) trehalose, 5.09% (w/w) histidine, and 1.05% (w/w) HCl;
d) 10% (w/w) anti-TSLP antibody or anti-TSLP antibody fragment, 15% (w/w) trileucine, 65.76% (w/w) trehalose, 7.66% (w/w) histidine, and 1.59% (w/w) HCl;
e) 10% (w/w) anti-TSLP antibody or anti-TSLP antibody fragment, 10% (w/w) trileucine, 73.73% (w/w) trehalose, 5.19% (w/w) histidine, and 1.08% (w/w) HCl;
f) 12.5% (w/w) anti-TSLP antibody or anti-TSLP antibody fragment, 10% (w/w) trileucine, 71.15% (w/w) trehalose, 5.25% (w/w) histidine, and 1.09% (w/w) HCl;
g) 20% (w/w) anti-TSLP antibody or anti-TSLP antibody fragment, 15% (w/w) trileucine, 55.44% (w/w) trehalose, 7.92% (w/w) histidine, and 1.64% (w/w) HCl;
h) 25% (w/w) anti-TSLP antibody or anti-TSLP antibody fragment, 15% (w/w) trileucine, 50.29% (w/w) trehalose, 8.05% (w/w) histidine, and 1.67% (w/w) HCl;
i) 25% (w/w) anti-TSLP antibody or anti-TSLP antibody fragment, 10% (w/w) trileucine, 58.26% (w/w) trehalose, 5.58% (w/w) histidine, and 1.16% (w/w) HCl;
j) 40% (w/w) anti-TSLP antibody or anti-TSLP antibody fragment, 15% (w/w) trileucine, 34.81% (w/w) trehalose, 8.43% (w/w) histidine, and 1.75% (w/w) HCl;
k) 50% (w/w) anti-TSLP antibody or anti-TSLP antibody fragment, 15% (w/w) trileucine, 24.5% (w/w) trehalose, 8.69% (w/w) histidine, and 1.81% (w/w) HCl; or
l) 50% (w/w) anti-TSLP antibody or anti-TSLP antibody fragment, 10% (w/w) trileucine, 32.48% (w/w) trehalose, 6.22% (w/w) histidine, and 1.3% (w/w) HCl.

66. The pharmaceutical composition of claim 49, wherein said composition is used in the preparation of a medicament.

67. The pharmaceutical composition of claim 66,
wherein the medicament comprises 0.5 mg to 16 mg of said anti-TSLP antibody or anti-TSLP antibody fragment.

68. The pharmaceutical composition of claim 66, wherein the medicament is in a powder form stored in a capsule or in a blister.

69. The pharmaceutical composition of claim 68, wherein said capsule or said blister is suitable for administration via inhalation.

70. A kit comprising the pharmaceutical composition of claim 49, and a device for delivering the pharmaceutical composition to a subject.

71. The kit of claim 70, wherein the device delivers the pharmaceutical composition in an aerosolized form.

72-73. (canceled)
